(19) United States
(12) Patent Application Publication
(10) Pub. No.: US 2004/0064014 A1
(43) Pub. Date: Apr. 1, 2004

(54) Devices and methods for assisting natural heart function

(76) Inventors: David B. Melvin, Loveland, OH (US); Jeffrey Radziunas, Wallingford, CT (US); Francisco M. Llort, Skillman, NJ (US); William Santamore, Medford, NJ (US); Scott J. Wolf, Bellevue, WA (US)

Correspondence Address: Kevin R Casey, RatnerPrestia, One Westlakes Berwyn Suite 301, PO Box 980, Valley Forge, PA, 19482-0980, US

(21) Appl. No.: 10/296,645

(22) PCT Filed: May 31, 2001
(86) PCT No.: PCT/US01/17637

(51) Int. Cl. A61F 2/00

(52) U.S. Cl. 600/37

(57) ABSTRACT

Devices and methods for treating a diseased heart including devices and methods for remodeling or reconfiguring a shape of a diseased heart, assisting in function of a diseased heart, and stabilizing such devices on a diseased heart. In some embodiments, the devices and methods include one or more segments for changing a shape of the heart or a portion thereof, and methods for using such devices and methods.

### FIELD OF THE INVENTION

This invention relates to devices and methods for assisting in the activation and operation of a living heart, including structures for mechanically deforming cardiac tissue such that the circulation of blood is maintained and assisting in movement of cardiac tissue during the cardiac cycle.

### BACKGROUND OF THE INVENTION

Various methods and devices have been proposed for altering the shape of a diseased heart chamber. None have yet proven practical and effective. The present invention addresses a number of new methods and devices to improve, or avoid the deficiencies of prior methods and devices.

### SUMMARY OF THE INVENTION

The present invention is directed to devices and methods for reconfiguring one or more chambers of a natural heart to reduce wall tension on the natural heart walls and/or for reconfiguring one or more structures such as valves, muscles, tendons or other structures of the natural heart, and/or to alter, improve or correct the anatomical structure of the natural heart so that the natural heart can function more efficiently or to correct other problems of the heart. In several embodiments, the segment or segments are adapted to lie adjacent the external surface of the natural heart in an unrestrained position, to cause an inward displacement of one or more locations of the external surface of the natural heart, and to prevent the natural heart from returning to44 the unrestrained position. In other embodiments, the segment or segments are internal to one or more chambers of the natural heart.

In one or more embodiments, the devices include one or more main segments that encircle a portion of or the entire natural heart at a selected location. The segments of the present invention are configured to provide differential pressure along a selected location of one or more chambers on the surface of the natural heart or a portion thereof by including rigid, semi-rigid and flexible segments or portions thereof, at different locations of the segment or segments of the devices on the natural heart, thereby displacing one or more chambers of the natural heart or a structure thereof (such as a heart valve, muscle, or tendon) and to prevent it from returning to its unrestrained configuration. Several elements such as the main segments or stabilizer/reconfiguration segments can be interchanged and combined with one another to form a device according to the present invention whereby these segments displace one or more positions of the natural heart and prevent the natural heart from returning to an unrestrained position.

The length and/or configuration of the devices or elements thereof according to the present invention can be adjusted by one or more adjustment and/or closure or locking mechanisms. Such adjustment and closure features include cables, chains, belts, straps, ratchets, blocks, telescoping elements, expandable elements such as a bellows, or screw mechanisms or similar mechanical or electromechanical devices, combined with or integral to the devices, and that allow adjustment of the devices or portions thereof according to the present invention during initial placement of the devices, and periodically after the devices have already been in place.

The devices according to the present invention can be stabilized and/or anchored in position with non-absorbable, partially absorbable, or fully absorbable protrusions; by rigid, semi-rigid or flexible strapping, tabs or curved portions of the segment; by reusable fasteners such as Velcro® or Velcro®-type fasteners; or by the shape or porosity of the segment itself. Stabilization features are adjustable during initial placement of the devices and periodically subsequent to placement of the devices.

The present invention also includes devices that assist the natural heart to function during one or more portions of the systolic and diastolic cycles. For example, the present invention includes a spring or spring-like mechanism that assist systolic and/or diastolic functions by exerting an outward or inward force on the inside or outside walls of the natural heart.

The present invention also includes methods for placing heart reconfiguration devices internal to the heart.

One or more of the devices or elements of specific embodiments shown and described herein can be used alone or in combination with other devices or elements thereof, and other devices not shown herein.

The present invention also provides devices and methods for treating cardiomyopathies that address and overcome the above-mentioned problems and shortcomings in the thoracic medicine art. The present invention also provides devices and methods for treating cardiomyopathies that minimize damage to the coronary circulatory, endocardium, and internal heart structures; devices and methods for treating cardiomyopathies that maintain the stroke volume of the heart; and devices and methods for treating cardiomyopathies that support and maintain the competence of the heart valves so that the heart valves can function as intended.

The present invention also provides devices and methods that increase the pumping effectiveness of the heart, and devices and methods for treating cardiomyopathies on a long term basis.

In one embodiment, the present invention provides devices and methods for treating cardiomyopathies that do not require removal of any portion of an existing natural heart. In another embodiment, the present invention provides devices and methods for treating dilated cardiomyopathies that directly reduce the effective radius of a chamber of a heart in systole as well as in diastole.

The devices of the present invention can be fixed to the heart in a manner which keeps the device in a desired location. In one or more embodiments, the present invention includes a stabilization system which employs rigid, semi-rigid, flexible belts or straps or harnesses. In one embodiment, the stabilization system or remodeling elements provide a site onto which cardiac transceivers or pacing leads may be secured which allows adding a plurality of transceivers or pacing leads to the heart at whatever spacing and arrangement may be desired.

### BRIEF DESCRIPTION OF THE DRAWINGS

FIG. 1A is a top cross-sectional view of a convex main segment on a heart;

FIG. 1B is a top cross-sectional view of a flat main segment on a heart;

FIG. 1C is a top cross-sectional view of a concave main segment on a heart;

FIG. 1D is a perspective view of a convex main segment on a heart;

FIG. 1E is a perspective view of a flat main segment on a heart;

FIG. 1F is a perspective view of a concave main segment on a heart;

FIG. 2A is a perspective view of a heart remodeling clasp including two main segments and apical and atrial segments, in an open configuration;

FIG. 2B is a perspective view of a heart remodeling clasp including two main segments and apical and atrial segments, in a closed configuration;

FIG. 3 is a perspective view of a heart remodeling clasp including two main segments and apical and atrial segments, in a closed configuration on a heart;

FIG. 4 is a top perspective view of a heart remodeling clasp including two main segments and apical and atrial segments, in a closed configuration on a heart;

FIG. 5A is a side perspective view of a main segment with a stabilizer/reconfiguration segment to support a valvular annulus of a heart;

FIG. 5B is a side perspective view of a main segment with a stabilizer/ reconfiguration segment to support the base of one or more papillary muscles;

FIG. 6 is a side cross-section view of a heart fitted with a stabilizer/reconfiguration segment to support a valvular annulus of a heart;

FIG. 7 is a perspective view of two adjustable heart stabilizer/reconfiguration segments attached to a main segment;

FIG. 8 is a perspective view of two adjustable heart stabilizer/reconfiguration segments attached to a main segment, on a heart;

FIG. 9A is a perspective view of two main segments and atrial and apical segments with pivot points to allow the segments to move with respect to one another;

FIG. 9B is a perspective view of the device of FIG. 9A on a heart;

FIG. 10A is a perspective view of a stabilizer/reconfiguration segment formed of a porous material;

FIG. 10B is a perspective view of a stabilizer/reconfiguration segment made of stays, adjustable by cables routed through openings in the stays and the heart stabilizing segments;

FIG. 11A is a perspective view of a stabilizer/reconfiguration segment made of stays, attached to two main segments;

FIG. 11B is a top cross-sectional view of another embodiment of a stabilizer/reconfiguration segment;

FIG. 12A is a side perspective view of a heart remodeling clasp including two main segments, an apical segment, an atrial segment and two stabilizer/reconfiguration tabs;

FIG. 12B is a side perspective view of another embodiment of a heart remodeling clasp including two main segments, an apical segment, an atrial segment and two stabilizer/reconfiguration tabs;

FIG. 13A is a side perspective view with phantom lines of the device in FIG. 12A;

FIG. 13B is a side perspective view of with phantom lines of the device in FIG. 12B;

FIG. 14A is a side perspective view of the device in FIG. 12A;

FIG. 14B is a side perspective view of the device in FIG. 12B;

FIG. 15A is a side cross-sectional view of a main segment with protrusions on the main segment;

FIG. 15B is a side cross-sectional view of the device in FIG. 15A in contact with heart tissue;

FIG. 15C is a top cross-sectional view of a main segment with moveable protrusions on the main segment;

FIG. 15D is a top cross-sectional view of the device in FIG. 15C in contact with heart tissue;

FIG. 16 is a perspective view of a main segment with moveable protrusions on a surface of the main segment;

FIG. 17 is a perspective view of a main segment including a multi-segmented, self-orienting plate;

FIG. 18A is a perspective view of an assembled main segment including multi-segmented, self-orienting plates;

FIG. 18B is a perspective view of one plate attached to a main segment, with movement of the plate shown by dotted lines;

FIG. 18C is an enlarged perspective view of one plate shown in FIG. 18A;

FIG. 19 is a perspective view of the device in FIG. 18A having a shell;

FIG. 20A is a perspective view of an alternative embodiment of a plate of a multi-segmented, self-orienting main segment;

FIG. 20B is a perspective view of multiple plates of FIG. 20A;

FIG. 20C is a perspective view of a main segment including multiple plates in FIGS. 20A and 20B;

FIG. 21A is a perspective view of another embodiment of a plate of a multi-segmented, self-orienting main segment;

FIG. 21B is a perspective view of a main segment including multiple plates in FIG. 21A;

FIG. 22 is a perspective view of part of a main segment including wire reinforcements;

FIG. 23 is an end view of main segment;

FIG. 24 is a top perspective view of reinforcement wires of a main segment with a zigzag configuration;

FIG. 25 is a perspective view of a series of reinforcement wires connected by one or more perpendicularly-mounted wire connectors;

FIG. 26 is a perspective view of an apical segment;

FIG. 27 is a perspective view of an atrial segment;

FIG. 28 is a perspective view of another embodiment of a main segment;

FIG. 29 is a view of an embodiment of a main segment capable of connecting with adjacent atrial or apical segments by a telescoping open channel joint;

FIG. 30 is a view of an embodiment of a main segment capable of connecting with adjacent atrial or apical segments by telescoping complementary interlocking grooves;

FIG. 31 is a perspective view of multiple segment plates or reinforcements of a main segment enclosed in a shell;

FIG. 32 is a perspective view of an embodiment of a spring mechanism including a bundle of spring wires linked by tethers;

FIG. 33 is a perspective side cross-section of a ventricle containing two spring mechanisms in FIG. 33, in the ventricle;

FIG. 34 is a side cross-section view of the spring mechanism of FIG. 32, within a ventricle;

FIG. 35 is a top cross-section view of two spring mechanisms of FIG. 32 within a ventricle, and two main segments remodeling the ventricle;

FIG. 36 is a top partial cross-section view of two spring mechanisms of FIG. 32 having coatings on the individual wires thereof, before and after tissue overgrowth;

FIG. 37A is a side perspective view of an apical coupling cap to be placed over the post tips of two spring mechanisms;

FIG. 37B is side perspective view of FIG. 37A, after placement of the apical coupling cap over the post tips;

FIG. 38 is a perspective view of an insertion sheath containing a spring mechanism of FIG. 32;

FIG. 39 is a perspective view of the device of FIG. 38 partially inserted into the apical portion of a ventricle;

FIG. 40 is a perspective view of one embodiment of deployment of the spring mechanism from the sheath shown in FIG. 38;

FIG. 41 is a top cross-section view of another embodiment of a spring mechanism in a ventricle and connected to two heart remodeling main segments;

FIG. 42 is a top cross-section view of another embodiment of a spring mechanism outside a ventricle and connected to two heart remodeling main segments;

FIG. 43 is a side cross-section view of another embodiment of a spring mechanism within a ventricle;

FIG. 44 is a top cross-section view of FIG. 43 and including certain structure of the heart;

FIG. 45 is a side cross-section view of another embodiment of the spring mechanism in a U-shaped configuration in a ventricle;

FIG. 46A is a perspective view of positioning of a tether connected to a main segment around a portion of the heart;

FIG. 46B is a side cross-section view of the tether of FIG. 46A surrounding a portion of the heart;

FIG. 47A is a perspective view of the main segment and attached tether in FIG. 46A with the main segment in place on the posterior of the heart;

FIG. 47B is a side cross-section view of the main segment and tether on a heart shown in FIG. 47A;

FIG. 48A is a perspective view of two main segments and one or more tethers being placed around a portion of the heart;

FIG. 48B is a side cross-section view of the main segments and one or more tethers on a heart shown in FIG. 48A;

FIG. 49A is a perspective view of two main segments and one or more tethers in place on a heart;

FIG. 49B is a side cross-section view of the main segments and one or more tethers on a heart shown in FIG. 49A;

FIG. 50A is a side view of a spacer between two main segments;

FIG. 50B is a side view of a spacer compressed between two main segments;

FIG. 51A is a side view of a spacer and two mains segments with a tether threaded through the spacer and main segments;

FIGS. 51B-E are additional embodiments of spacers for placement between two main segments;

FIG. 52 is a perspective view of a remodeling device including two main segments, one or more tethers, and an adjustment canister on a heart;

FIG. 53 is a perspective view of the device in FIG. 52 off the heart;

FIG. 54A is a side view of another embodiment of a main segment with hinged shoulders (in an open position) and a tether running through the main segment;

FIG. 54B is a side view of the main segment in FIG. 54A with the hinges of the main segment in a closed position;

FIG. 54C is a partial perspective view of the main segment in FIG. 54A having slightly wider elements and with the hinges in an open position;

FIG. 54D is a partial perspective view of the device in FIG. 54C with the hinges in a closed position;

FIG. 55 is a perspective view of an embodiment of the present invention including a main segment, a shoulder segments, and adjustable closures;

FIG. 56 is a top view of an stabilizer/reconfiguration segment;

FIG. 57A is a perspective view of a clip used to fasten a stabilizer/reconfiguration segment on the device of FIG. 55;

FIG. 57B is a side view of a clip of FIG. 57A;

FIG. 58 is a top view of another embodiment of a stabilizer/reconfiguration segment;

FIG. 59A is a perspective view of another embodiment of type of clip used to fasten an stabilizer/reconfiguration segment on the device of FIG. 55;

FIG. 59B is a side view of the clip in FIG. 59A;

FIG. 59C is a top view of the clip in FIG. 59A;

FIG. 60A are perspective and top, respectively, views of a pin used to secure a clip to a stabilizer/reconfiguration segment;

FIG. 61 is a partial perspective view of the device in FIG. 55;

FIG. 62 is a partial perspective view of the device in FIG. 55;

FIG. 63A is a top perspective view of the device of FIG. 55 including two main segments with pads attached thereto and the stabilizer/reconfiguration segments in FIGS. 56 and 58 attached thereto;

FIG. 63B is side perspective view of the device shown in FIG. 63A;

FIG. 64A is a side view of a device in FIG. 55 including two main segments having multi-segmented plates thereon;

FIG. 64B is a perspective view of the device in FIG. 64A;

FIG. 65 is a top cross-sectional view of multiple positions of main segments on a heart;

FIG. 66 is a top view of the device in FIG. 65 placed on a heart and including two stabilizer/reconfiguration segments;

FIG. 67 is a side view of a main segment and a stabilizer/reconfiguration segment on a heart;

FIG. 68 is a perspective view of a U-shaped remodeling device including multiple stabilizer/reconfiguration segments and pacing leads;

FIG. 69A is a cross-sectional view of a main segment encased in a suturable material;

FIG. 69B is a cross-sectional view of a main segment encased in a suturable material;

FIG. 70 is a perspective view of the device in FIG. 69A and having one large stabilizer/reconfiguration segment and pacing leads;

FIG. 71 is a perspective view of the device in FIG. 69 and having multiple relatively narrow stabilizer/reconfiguration segments and pacing leads;

FIG. 72 is a cross-sectional view of a ball snap clamping mechanism used to attach a stabilizer/reconfiguration segment to a main segment;

FIG. 73A is a cross-section view of placing an umbrella-like anchored tensioning device in a catheter in a ventricle;

FIG. 73B is a cross-section view of the insertion of the anchored device in FIG. 73A;

FIG. 74A is a cross-section view of an anchored tension device in a ventricle with tensioning cords;

FIG. 74B is a cross-section view of the device of FIG. 74A in place;

FIG. 75A is the device in FIG. 73A, including a clamshell like anchor before placement;

FIG. 75B is the device in FIG. 73B, including a clamshell like anchor after placement;

FIGS. 76A-C are side views of a main segment and stabilization protrusions before, during and after, respectively, placement of the device on a heart wall

FIGS. 77A-C are side cross-section views of a main segment having absorbable stabilization protrusions including a non-resorbable insert, before, during and after, respectively, absorption of the protrusion on a heart wall;

FIGS. 78A-B are side cross-section views of a main segment including tensions stabilization protrusions before and after, respectively, deployment of the protrusions;

FIGS. 79A-B are side cross-section views of a main segments including multiple longitudinally aligned stabilization protrusions;

FIGS. 79C-D are side cross-section views of a main segment including multiple transversely aligned stabilization protrusions;

FIGS. 80A-B are perspective and cross-section views of another embodiment of stabilization protrusions

FIG. 81A is a side view of the stabilization protrusion of FIGS. 80A-B, being placed in a main segment;

FIG. 81B is a side cross-section of the stabilization protrusion in FIG. 81A, in a main segment in FIG. 81A placed on a heart wall;

FIGS. 82A-B are side cross-section views of the device in FIG. 81B during and after, respectively, absorption of a portion of the stabilization protrusion;

FIG. 83 is a perspective view of a flexible sheath for covering one or more segments of heart remodeling devices of the present invention;

FIG. 84A is a perspective view of the flexible sheath in FIG. 83 in position around a heart;

FIG. 84B is a side cross-section view of the flexible sheath in position in FIG. 84A;

FIGS. 85A-85D are perspective views of rigid segments to be placed in the sheath in FIG. 83 to form a heart remodeling device;

FIGS. 86A-D are side cross-section views of placing multiple interlocking segments in the sheath in FIG. 83;

FIG. 86E is a side view of interlocking rigid segments in FIGS. 86A-D;

FIG. 86F is a cross-section view of the device in FIG. 86D and having a final segment encased in a sheath in place on an end of the device;

FIGS. 86G-H are cross-section views before and after, respectively, interlocking the final segment in FIG. 86F into place;

FIG. 87 is a perspective view of another embodiment of a main segment the curvature of which can be changed;

FIG. 88 is a perspective view of the individual blocks and pins comprising the device in FIG. 87;

FIG. 89 is a side cross-section view of a main segment including the structure in FIG. 87;

FIG. 90 is an alternative embodiment of the mechanism in an end block of the device in FIG. 89, for changing the curvature of the main segment;

FIGS. 91A-B are a side cross-section views of another embodiment having a single cable for changing the curvature of a main segment, in straight and curved positions, respectively;

FIGS. 91C-D are side cross-section views of another embodiment having two cables for changing the curvature of a main segment, in straight and curved positions, respectively;

FIGS. 92A-B are side cross-section views of another embodiment having one cable for changing the curvature of a main segment including one or more notched edges;

FIGS. 93A-B are perspective views of a series of telescoping segments in curved, and in curved and shortened, respectively, positions;

FIG. 94 is a perspective view of another embodiment for changing the length of a segment including telescoping elements;

FIG. 95 is a cross-section view of a series of telescoping elements having a slightly longer and narrower configuration;

FIG. 96 is a cross-section view of another embodiment of a segment including telescoping elements, a cable and threaded ends;

FIG. 97 is a perspective view of another embodiment for hydraulically adjusting the length or curvature of a segment;

FIG. 98 is a cross-section of another embodiment of changing the length of a segment including telescoping elements and piston bars between the telescoping elements;

FIGS. 99A-C are three descriptions of changing the curvature and/or length of segments according to the invention;

FIG. 100 is a schematic of placement in a body of an adjustment canister for adjusting the distance of two main segments and/or stabilizer/reconfiguration segments;

FIGS. 101A-E are perspective views of a control mechanism including covering caps, push rods and screw assembly, for locally or remotely adjusting the distance between an inside surface and an outside surface of a main segment, or the distance between two opposing main segments,

FIG. 102 is a perspective view of another embodiment of an adjustment mechanism for locally or remotely adjusting the distance between an inside surface and an outside surface of a main segment, or the distance between two opposing main segments;

FIG. 103 is a perspective view of another embodiment of an adjustment mechanism for locally or remotely adjusting the distance between an inside surface and an outside surface of a main segment, or the distance between two opposing main segments;

FIG. 104 is a perspective view of another embodiment of an adjustment mechanism including a diaphragm and a syringe, for locally or remotely adjusting the distance between an inside surface and an outside surface of a main segment, or the distance between two opposing main segments;

FIG. 105A is a side view of another embodiment of an adjustment mechanism including an electric or magnetic drive and a transcutaneous coupling, for locally or remotely adjusting the distance between an inside surface and an outside surface of a main segment, or the distance between two opposing main segments;

FIG. 105B is a side view of another embodiment of an adjustment mechanism including a solenoid or permanent magnet driven by a hydraulic pump and a transcutaneous coupling, for locally or remotely adjusting the distance between an inside surface and an outside surface of a main segment, or the distance between two opposing main segments;

FIGS. 106A-C are cross-section views of several embodiments of a main segment including an expandable membrane between an inner surface and an outer surface of the main segment, or for moving an inner surface of the main segment relative to an outer surface of the main segment;

FIG. 107 is a cross-section views of another embodiment of a main segment including an screw mechanism for moving an inner surface of the main segment relative to an outer surface of the main segment;

FIG. 108 is another embodiment of the device of FIG. 108 including a rotatable cable for advancing the screw;

FIGS. 109A-B are side cross-section views of a main segment including a lever operated by a pull cord for moving an inner surface of the main segment relative to an outer surface of the main segment, in closed and open positions, respectively;

FIGS. 110A-B are side cross-section views of a main segment including another embodiment of a lever operated by a screw cable for moving an inner surface of the main segment relative to an outer surface of the main segment, in closed and open positions, respectively;

FIGS. 111A-B are side cross-section views of a main segment including a hydraulic bellows for moving an inner surface of the main segment relative to an outer surface of the main segment, in closed and open positions, respectively;

FIGS. 112A-B are side cross-section views of a main segment including a hydraulic piston for moving an inner surface of the main segment relative to an outer surface of the main segment, in closed and open positions, respectively;

FIGS. 113A-B are cross-section views of another embodiment of a main segment including an expandable fluid between an inner and outer surface of the main segment, for moving an inner surface of the main segment relative to an outer surface of the main segment, in closed and open positions, respectively;

FIGS. 114A-B are cross-section views of another embodiment of a main segment including movable screw operated shims between an inner and outer surface of the main segment, for moving an inner surface of the main segment relative to an outer surface of the main segment, in closed and open positions, respectively;

FIG. 115 is an end view of another embodiment of an apical stabilization cap;

FIG. 116 is a side view of the device in FIG. 115;

FIG. 117 is a top perspective of the device in FIG. 115;

FIG. 118 is a bottom perspective of the device in FIG. 115;

FIG. 119 is perspective view of another embodiment of an apical stabilization cap;

FIGS. 120A-B are perspective and side views of an apical stabilization cap including a guide channel;

FIGS. 121A-D are perspective and side views of several embodiments of seams of the apical stabilization cap in FIG. 119 or FIGS. 120A-B;

FIG. 122 is a side view of the apical stabilization cap in FIG. 119 on a heart;

FIG. 123 is partial view in FIG. 122 showing pleats or tucks for circumferential size adjustment of the cap;

FIG. 124 is a perspective view of a main segment stabilized on a heart with an apical stabilization cap;

FIG. 125 is a perspective view of a another embodiment of an apical stabilization cap with four circumferential purse strings for adjusting the shape and/or size of the cap;

FIG. 126 is a partial perspective view of two main segments and one or more cables connecting the segments;

FIG. 127 is an enlarged perspective view of a clamping mechanism for clamping cables to the main segment;

FIG. 128A is a top view of the clamping mechanism in FIG. 127;

FIG. 128B is a cross-section view of the clamping mechanism in FIG. 127;

FIG. 129 is a top perspective view of a clamp off the main segment;

FIG. 130 is a longitudinal cross-section of the clamp in FIG. 129;

FIG. 131 is an enlarged view of a longitudinal cross-section of a portion of the clamp in FIG. 130;

FIG. 132 is a perspective view of a clamping mechanism on a main segment;

FIG. 133 is a cross-sectional view of the center portion of the clamping mechanism in FIG. 132;

FIGS. 134-137 are perspective or side views of another embodiment of the a heart remodeling device and a remote adjusting mechanism, including a clamping mechanism;

FIG. 138 is an enlarged side view of a portion of a main segment having three purse string or cable holes;

FIG. 139 is an enlarged perspective side view of the clamping mechanism in FIG. 138;

FIG. 140 is an enlarged perspective view of the clamping mechanism shown in FIG. 138;

FIGS. 141-142 are side and perspective views of another embodiment of a main segment;

FIG. 143 is an enlarged view of the main segment of FIGS. 141-142 on a rigid rod;

FIG. 144 is a perspective view of a main segment and a stabilizer/reconfiguration segment on a heart;

FIG. 145 is perspective view of the device in FIG. 144 on a heart with the posterior portion of the device in partial phantom lines;

FIG. 146 is a top view of the base of a heart, with the device in FIG. 145;

FIG. 147 is a perspective view of two main segments and two stabilizer/reconfiguration segments attached to the main segments;

FIG. 148 is a top view of the device on the heart shown in FIG. 147 where the heart wall is enlarged below the stabilizer/reconfiguration segments;

FIG. 149A is a perspective view of another embodiment of a stabilizer/reconfiguration segment;

FIG. 149B is a perspective view of another embodiment of a stabilizer/reconfiguration segment;

FIG. 150 is a side cross-section view of a heart fitted with a stabilizer/reconfiguration segment to support a valvular annulus of a heart;

FIG. 151 is an enlarged perspective view of a portion of a main segment including a sheath and stabilization protrusions;

FIG. 152 is an enlarged perspective view of another embodiment of a main segment including a covering sheath;

FIG. 153 is a cross-section view of the main segment of FIG. 152 with stabilization protrusions;

FIG. 154 is a perspective view of two main segments, one or more tethers, stabilization protrusions and a covering sheath over the device;

FIG. 155 is a perspective view of two main segments, one or more tethers, stabilization protrusions and an alternative embodiment of a covering sheath over the device;

FIG. 156 is a cross-section view of the main segment in FIG. 155 after placement on a heart wall;

FIG. 157 is a cross-section view of the main segment in FIG. 155 after movement along the direction the arrow;

FIG. 158 is a cross-section view of the main segment in FIG. 155 after placement for a period of time allowing tissue ingrowth into the sheath and with secured edges;

FIG. 159 is a perspective view of a dilator body and dilator nose for placing devices according to the present invention;

FIG. 160 is an enlarged view of the dilator body and dilator nose in FIG. 159;

FIGS. 161A-D are perspective and side views of a dilator clasp adapter, for connection to a dilator body and, for example, a main segment;

FIG. 162 is a cross section showing an endoscope surrounding a portion of the heart;

FIG. 163 is an enlarged view through the endoscope in FIG. 162 as it moves to a site of perforation of the pericardium;

FIG. 164 is a perspective view of a biting forceps grasping and opening a hole in a portion of the pericardium;

FIG. 165 is a cross-section view a tether or guide wire advanced through a hole in the pericardium, around the heart, and back out through the site of entry, and the endoscope leaving the field of view;

FIG. 166 is a cross-section view of a dilator body advanced over a tether or guide wire surrounding a heart;

FIG. 167 is a cross-section view of the dilator body and tether or cable in FIG. 166, and showing a dilator clasp adapter having an end of main segment inserted therein;

FIG. 168 is a cross-section of a dilator body advancing a main segment into position on the posterior portion of the heart;

FIG. 169 is a cross-section showing one end of second main segment threaded through an end of the tether or guide wire before placement of the second main segment on an anterior portion of the heart;

FIG. 170 is a cross-section showing a second end of a tether threaded through a second end of the second main segment before placement of the second main segment on the posterior portion of the heart;

FIG. 171 is cross-section view of the device in FIG. 170 on the heart;

FIG. 172 is a perspective view of a heart with one side of Velcro® fastener having alternating elastic strips, attached to the heart tissue;

FIG. 173 is an enlarged perspective view of a main segment with a second side of a of Velcro® fastener having alternating elastic strips attached thereto; and

FIG. 174 is a cross-section of a heart wall and an attached structure (such as a main segment), wherein the structure is attached with a Velcro® fastener having alternating sections of elastic material.

### DETAILED DESCRIPTION OF THE INVENTION

The invention is described with reference to the drawings. The figures of the drawings are illustrative rather than limiting and are included to facilitate the explanation of the invention.

Remodeling Support Device

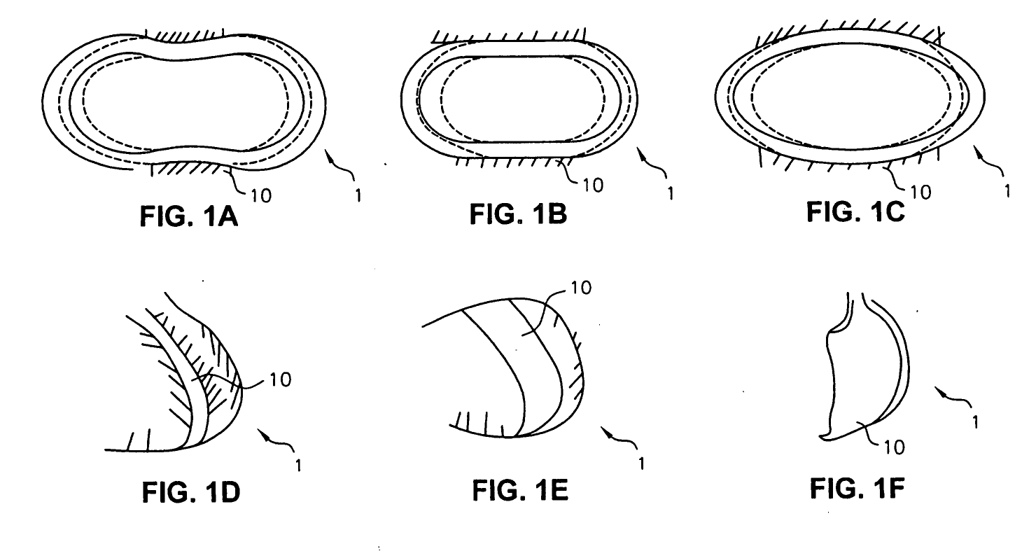

The invention provides a segment that supports and reconfigures the heart. As shown in FIG. 1A, a main segment 10 can be modeled to a heart 1 having actual human cardiac heart failure (CHF) dimensions. Preferably, the main segment 10 is configured and positioned on the heart to provide a contact pressure of about 1.4 to about 0.7 times (+/−0.2) the cavitary pressure.

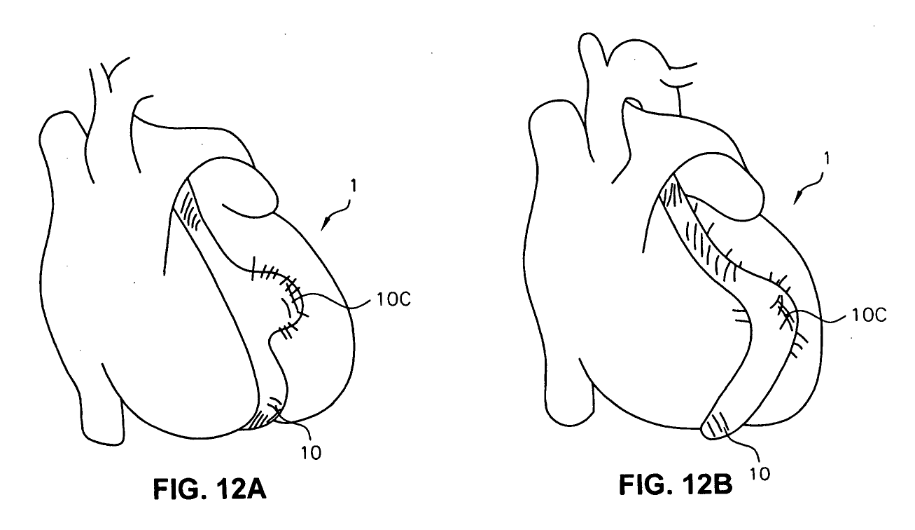

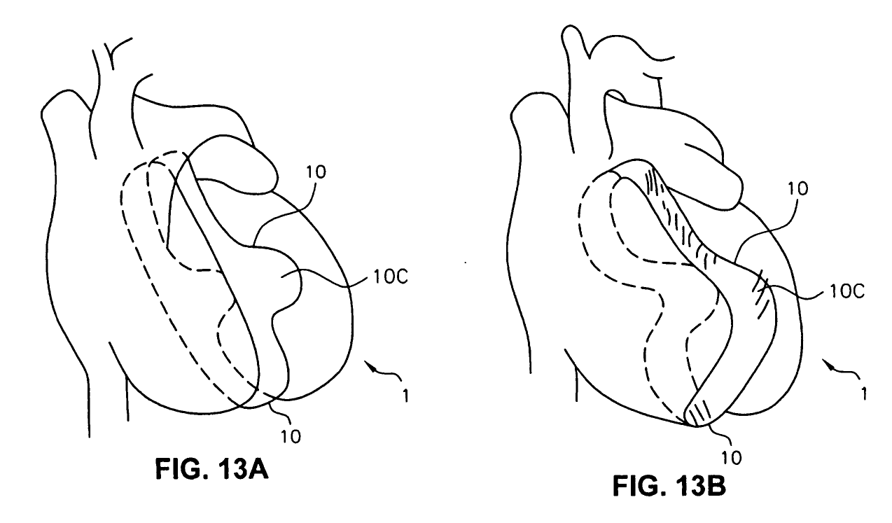

Main segment 10 of the invention can have many differing shapes, depending, for example, on the condition being treated and the size and shape of the heart. The cross section of the segment can have, for example, a convex shape toward the heart (as shown by main segment 10 in FIG. 1A), flat shape (as shown in FIG. 1B as main segment 10), swan shape (as shown in FIG. 12B and 13B), elliptical shape, concave shape (as shown by main segment 10 in FIG. 1C), or a combination thereof. FIGS. 1D, 1E, and 1F show main segments 10 of FIGS. 1A, 1B, and 1C, respectively, placed on a human heart 1.

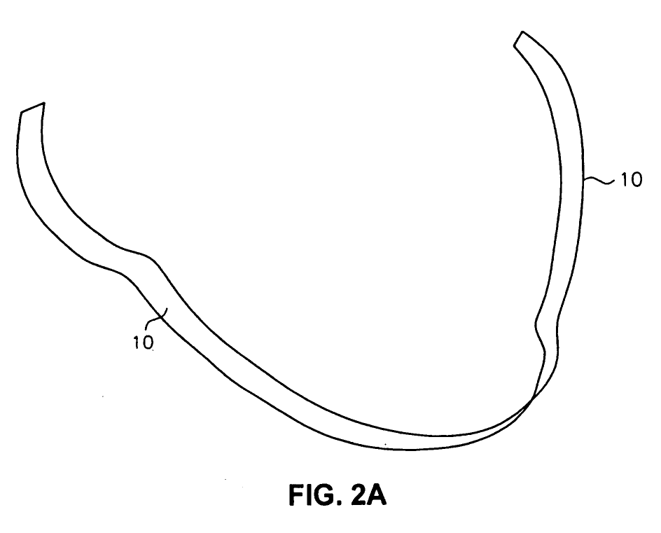

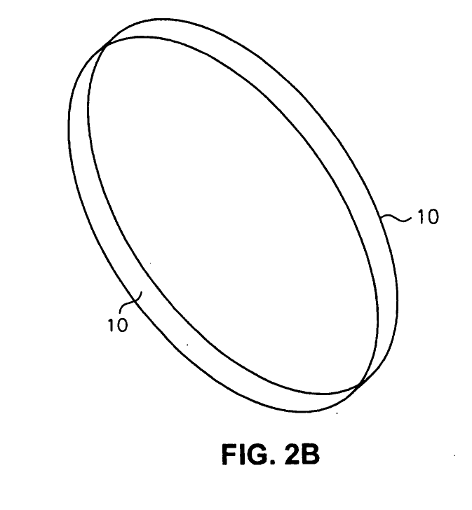

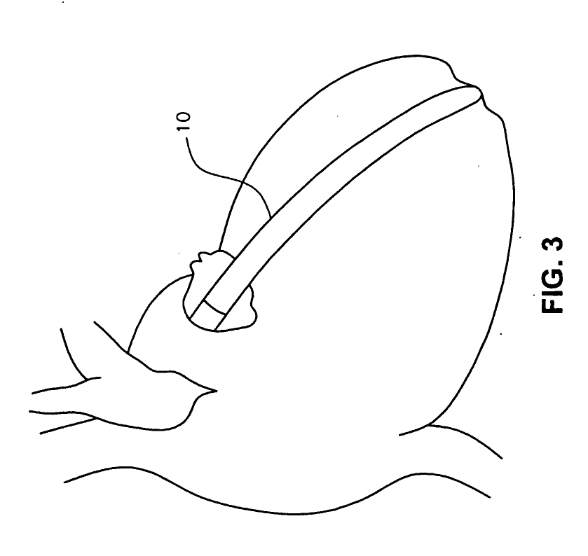

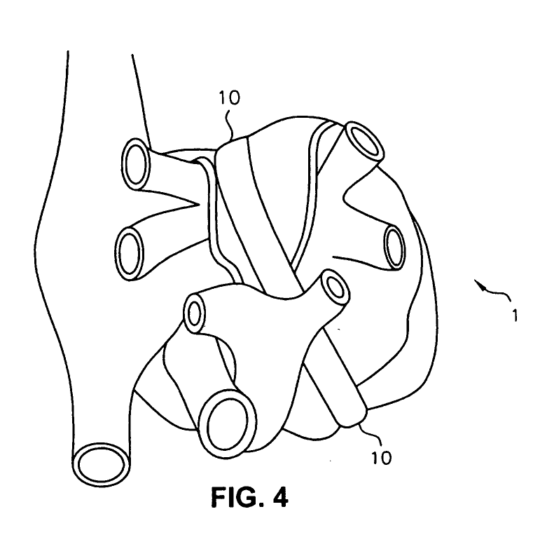

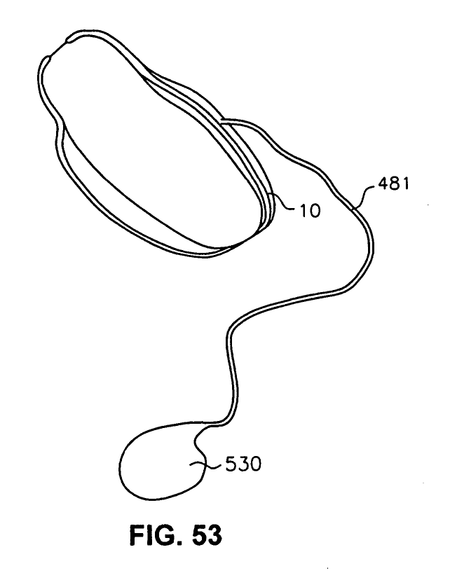

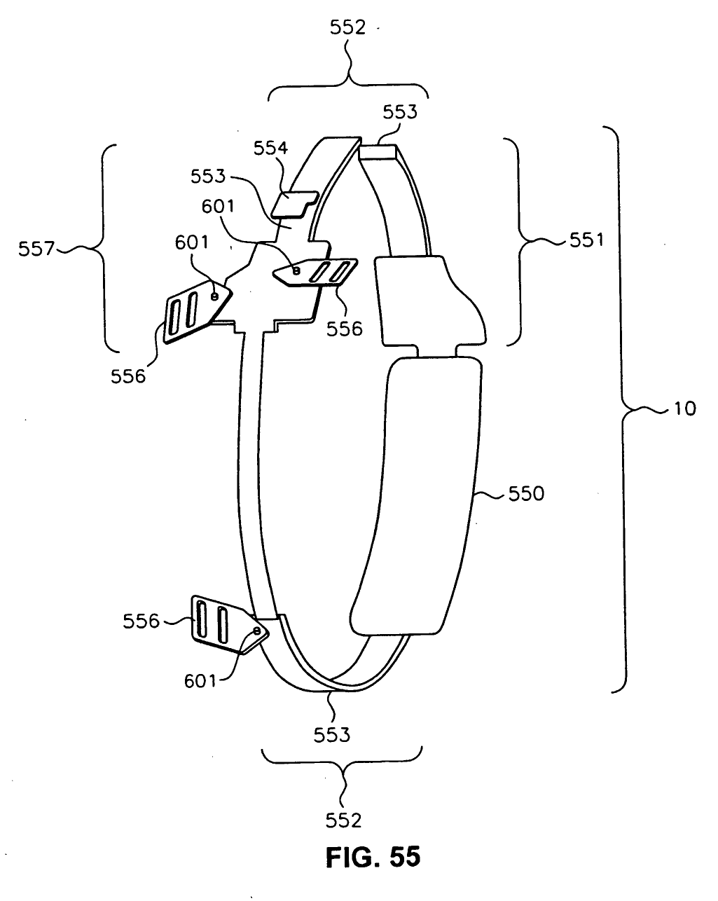

In addition, main segment 10 can have, for example, an O-shaped configuration such as main segment 10 shown in FIGS. 2, 2A, 2B, and 4. In FIG. 2A, main segment 10 is shown in an open configuration that is closed to form an O-shaped configuration around the natural heart or a portion thereof, as shown in FIGS. 2B, 3, and 4. Main segment 10 can also have adjustment mechanisms for adjusting the size (for example, length and width) and shape (for example, curvature) of the main segment with respect to the heart, including, but not limited to, the adjustment mechanisms shown, for example, in FIGS. 53, 55,62, 87-98. In some embodiments of the devices according to the present invention, up to 30% or more reduction in effective radius (e.g., endocardial or midwall radius) is achieved at initiation of systole.

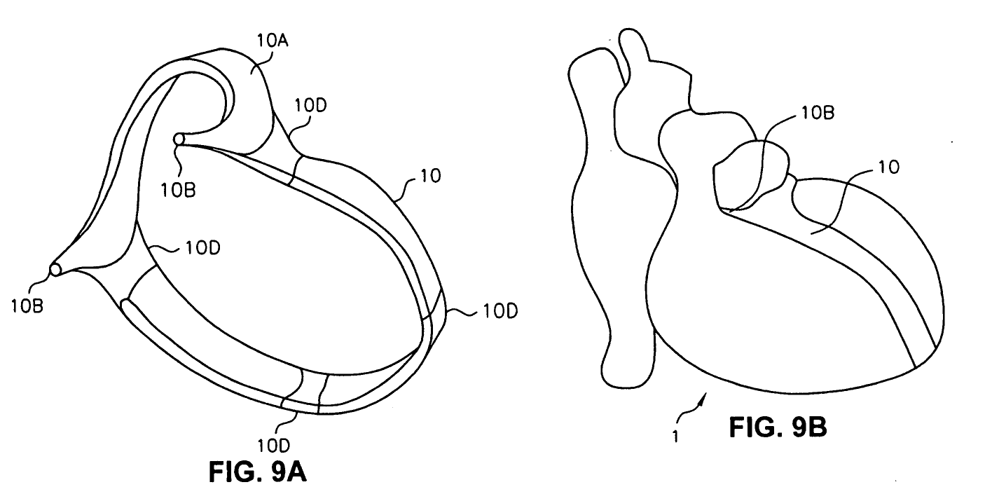

Referring again to FIG. 4, in one embodiment, the O-shaped device is positioned under the pulmonary artery root into the transverse sinus, then through the pericardial reflection and, then into the oblique sinus between the left and right pulmonary veins. In one embodiment according to FIGS. 2A, 3, and 4, and other embodiments of an O-shaped device, spontaneous systolic torsion is permitted by four discrete pivot points located on the device, such as is shown in FIG. 9A as pivot points 10d as more fully described in U.S. patent application Ser. No. 09/326,416, which is hereby incorporated by reference. The pivot points may be covered by a tough continuous elastomeric skin.

It is thought that some embodiments according to the present invention work because ventricular wall stress produced by a given intracavitary pressure is altered in direct proportion to the local radius of curvature or, alternatively stated, intracavitary ventricular pressure required to achieve a given wall stress is altered in inverse proportion to the local radius of curvature.

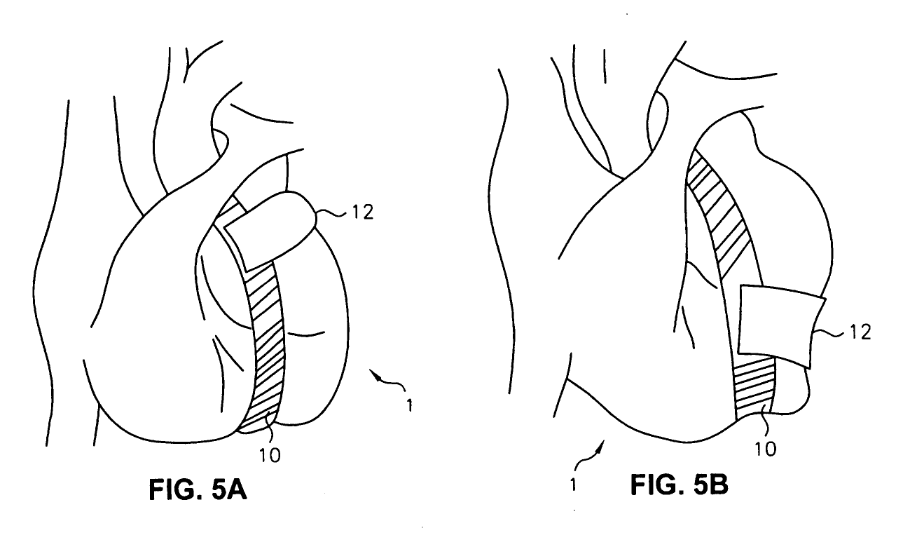

The present invention also provides a stabilizer/reconfiguration segment 12 (as shown for example in FIGS. 5A, 5B, 6, 7, 8, and 144-147) that stabilizes main segment 10 on heart 1 and/or supports and reconfigures part of the outside of heart 1 in one or more regions, for example, the region of the mitral or tricuspid valve apparatus in order to improve or eliminate reverse flow through those valves. In one embodiment, the present invention solves regurgitation (also known as insufficiency or incompetence) of the mitral valve or tricuspid valve of the heart. This is a condition in which the leaflets of the valve(s) fail to coapt sufficiently to halt backward flow of blood from a left or right ventricle of the heart to its respective atrium during contraction.

Stabilizer/reconfiguration segment 12 can be either a stand-alone device attached to treat the heart (e.g., valvular disease or separation caused by other heart disease), or used in combination with other heart treatment devices. This device is designed to fit adjacent to and support part of the external surface of the heart for the purpose of aiding mitral or tricuspid closure.

Preferably, stabilizer/reconfiguration segment 12 can be placed without use of cardiopulmonary bypass, without opening any cardiac chamber, and on a beating heart. Central anchoring of the stabilizer/reconfiguration segment 12 to a ventricular remodeling clasp including main segment 10, or other structure fixed to the ventricular wall, is expected to render the resulting repair more durable, better control valve shape, and be able to have an option of including a step of manipulating papillary muscle base position.

FIGS. 5A and 6-11B illustrate stabilizer/reconfiguration segment 12 for stabilizing main segment 10 on heart 1 and/or reconfiguring a portion of heart 1 that supports the valvular annulus of heart 1, directly or indirectly, by fitting around and supporting an outer margin of the junction between the atrium and ventricle, and/or the region thereof, of either the left or right side of the heart. In one embodiment, stabilizer/reconfiguration segment 12 exerts force upon the epicardium of the heart overlying the region of the junction between the left or right atrium and the ipsilateral ventricle (including the contiguous left or right atrial wall, and/or the contiguous left or right ventricular wall, and the coronary arteries and cardiac veins in the region), so that force is transmitted through these structures to the parts of the mitral or tricuspid annulus supporting the mural leaflets (posterior leaflet of the mitral valve and/or both the anterior and posterior leaflet of the tricuspid valve).

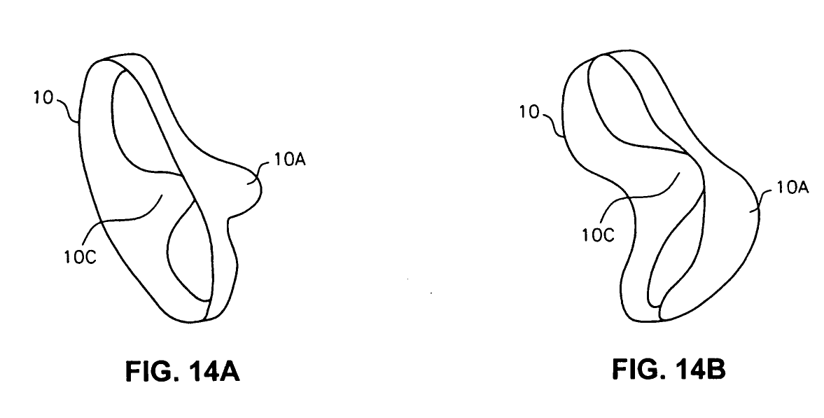

FIGS. 12A, 12B, 13A, 13B, 14A and 14B illustrate a device including main segment 10 having portions (extension segments) 10a, or 10c that support the base of one or more papillary muscles of either the mitral and/or tricuspid valve. In one embodiment, the device according to the present invention exerts force upon the epicardium overlying the region of the base of the papillary muscles in either ventricle.

It should be appreciated that each of the elements of the invention can be combined to achieve a desired outcome. For example, a structure intended to remodel the mitral valve may be mutually anchored to a structure intended to remodel the tricuspid valve.

Main segment 10 can be open-shaped, such as a ring, band, or collar structure, designed to fit around and support the outer margin of either (i) the junction between the atrium and ventricle and/or a region thereof and/or (ii) a portion of the ventricular wall overlying papillary muscle bases, of either the left or right side of the heart. Main segment 10 can be designed to be connected and supported at either end by attachment to one or more relatively stationary structures.

Main segment 10 can also have one or more portions such as extension segments 10a shaped for stabilization and/or support of the main segment 10 adjacent the heart 1, as shown in FIGS. 9A and 9B. In one embodiment, extension segment 10a is a tab-shaped, generally curved member, designed to be connected and supported at one end by another relatively stationary structure. Main segment 10 can also include one or more discrete pivot segments, shown in FIG. 9A as pivot segments 10d, which can provide low resistance to deformation in a direction perpendicular to the epicardial surface of the heart and can preserve freedom of movement for spontaneous systolic torsion as the heart expands and contracts.

The embodiments shown in FIGS. 1A-14B can include one or more adjustable stabilizer/reconfiguration segment 12 to stabilize (e.g., laterally stabilize) main segment 10 adjacent heart 1. One example of this stabilization is shown in FIG. 5A with main segment 10 being stabilized by stabilizer/reconfiguration segment 12. Stabilizer/reconfiguration segment 12 optionally can be shaped, sized, and configured so as to reconfigure the heart or a heart valve. More specifically, stabilizer/reconfiguration segment 12 can be used as shown in FIGS. 5A and 5B to cause a reconfiguration (e.g., valve remodeling) of the heart 1. The size, shape, and placement of the stabilizer/reconfiguration segment 12 can be varied depending on intended use. For example, the stabilizer/reconfiguration segment 12 can be used simply as a stabilizing band that passes around the opposite side of the heart (e.g., at least part of the right ventricle and/or atrium in the case of a member supporting the mitral valve) to maintain placement of one or more main segments 10 on the heart.

Stabilizer/reconfiguration segment 12 can be formed of numerous materials for stabilizing or supporting main segment 10. In addition, stabilizer/reconfiguration segment 12 can be adjustable as to total length and/or shape, by using, for example, a cord or cable traction, cable torsion, or other means applied directly to stabilizer/reconfiguration segment 12. Furthermore, adjustable stabilizer/reconfiguration segment 12 can be adjusted by means of one or more strings such as purse-strings where stabilizer/reconfiguration segment 12 is totally or partially flexible, or by telescoping of its parts where totally rigid. Such telescoping, in turn, can be driven, for example, by cable tension, hydraulic fluid injection/withdrawal, or turning of threaded members. In addition, stabilizer/reconfiguration segment 12 can be fixed centrally to one or more main segments 10 with sufficient stability to form a cantilever structure by which apically or basally-directed force components of heart-contact pressure serve to stabilize the clasp position in the apico-basal direction.

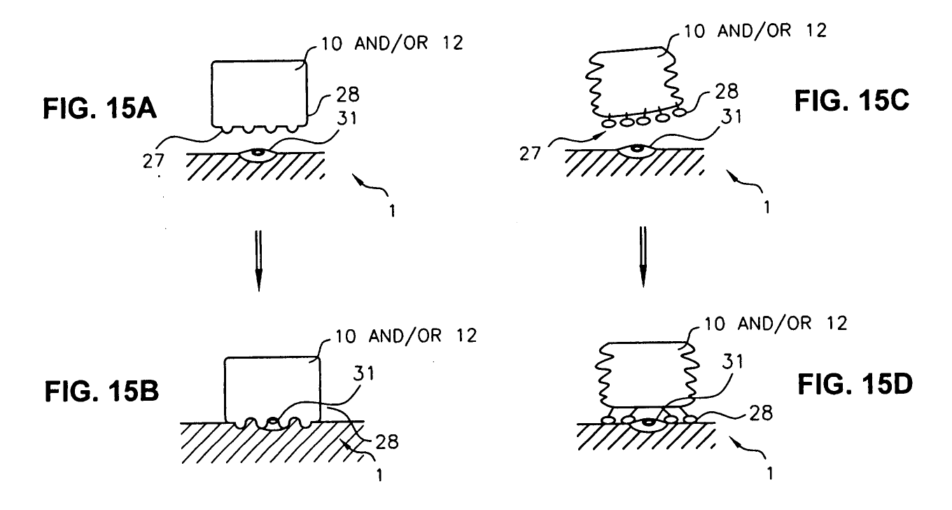

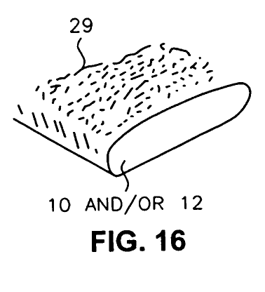

Main segment 10 and/or stabilizer/reconfiguration segment 12 can also have a heart-contacting surface 27 that is, for example, a solid surface, multiply perforated, such as a net or mesh (shown for example in FIGS. 69a, 69b, 153, 154, and 155), or a combination thereof. In one embodiment, heart contacting surface 27 may be a fluid filled (e.g., gel filled) or ‘potting’ filled pad, or a surface studded with bumps 28 or beads 29, as shown in FIGS. 15A, 15B, 15C, 15D and 16. FIGS. 15A, 15B, 15C, 15D, and 16, illustrate a cross section or perspective views of main segment 10 and/or stabilizer/reconfiguration segment 12 having bumps 28. In one embodiment, bumps 28 or beads 29 are roughly hemispheric or semi-hemispheric, fixed projections having a diameter of about 2 to about 2.5 mm, that are spaced about 2 to about 2.5 mm from one another, as shown in FIGS. 15A and 15B. Surface 27 may also have, for example, beads 29 that float, i.e., are attached to the surface and are movable with respect to the surface, as shown in FIGS. 15C and 15D. Preferably the moveable beads 29 have a diameter of about 1.5 to 2 mm and are tethered about 2.5 to about 3 mm apart. As shown in FIGS. 15A, 15B, 15C, and 15D, main segment 10 and/or stabilizer/reconfiguration segment 12 may be brought into contact with a section of natural heart 1 that has a traversing coronary artery 31 near the surface. Artery 31 moves slightly to nestle between beads 29 or bumps 28 due to its own intrinsic mobility. In the embodiment with floating bumps 28 or beads 299, bumps 28 and beads 29 may also move to accommodate positioning of artery 31.

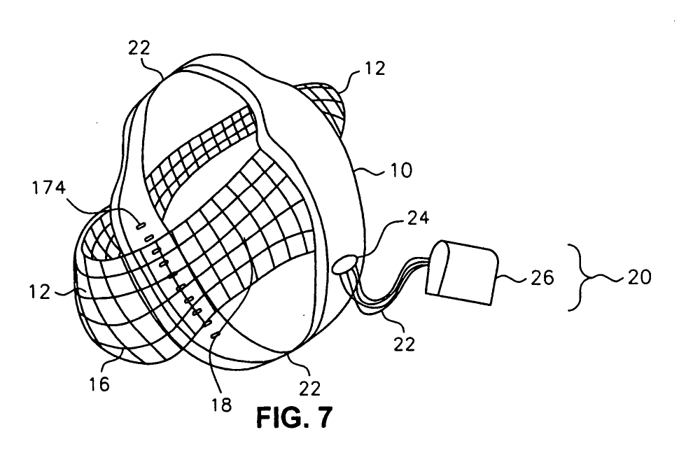

As shown in FIG. 7, the stabilizer/reconfiguration segment 12 can be formed of a mesh framing 16 having openings 18. Mesh framing 16 is flexible, rigid, or a combination thereof. Factors determining the desired flexibility or rigidity of the stabilizer/reconfiguration segment 12 include valve remodeling, facilitating coaptation of mural and non-mural leaflets, countering displacement of papillary muscle bases, and minimizing cyclic compressive or tensile stress at heart-contacting surfaces. Stabilizer/reconfiguration segment 12 can be made of, for example, a fabric material such as a porous or mesh material.

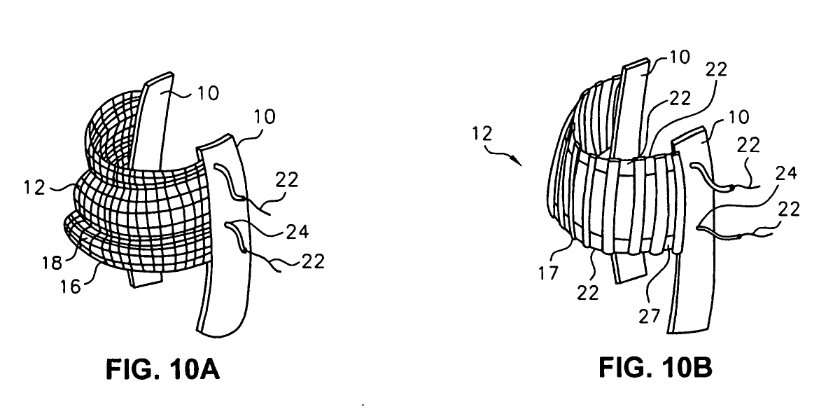

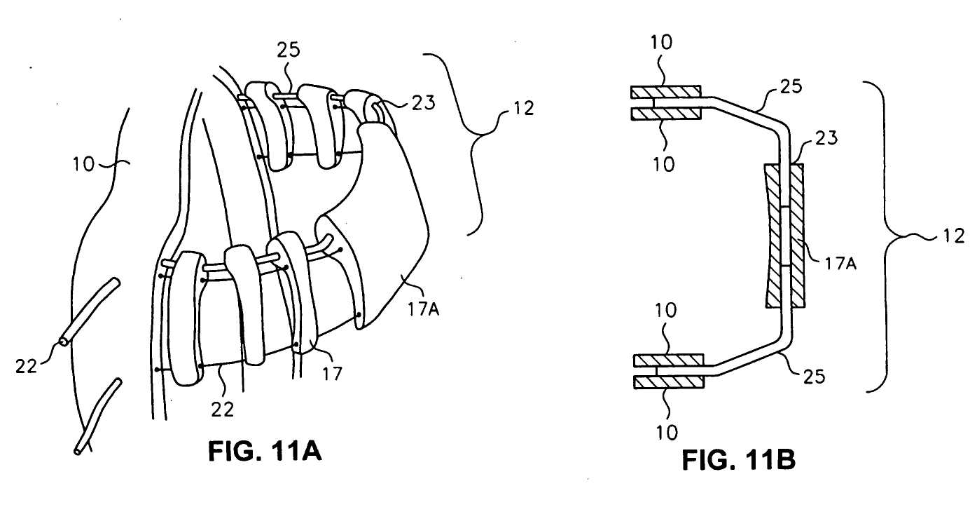

Stabilizer/reconfiguration segment 12 can also include, as illustrated in FIG. 10B, one or more bars or stays 17 connected to one another via one or more strings or cables 22. FIG. 10A illustrates that in embodiments where stays 17 are not used, adjustment of stabilizer/reconfiguration segment 12 may result in uneven tightening of the drawstrings. In one embodiment, each stay 17 can be identical in size and shape, as shown in FIG. 10B, or one or more of the stays 17 can have different sizes and shapes to optimize stability and/or support, such as stay 17a illustrated in FIG. 11A. Stays 17 can be rigid, semi-rigid, or a combination thereof. In addition, stays 17 can be curved, straight, or a combination thereof, to accommodate the size and shape of the heart.

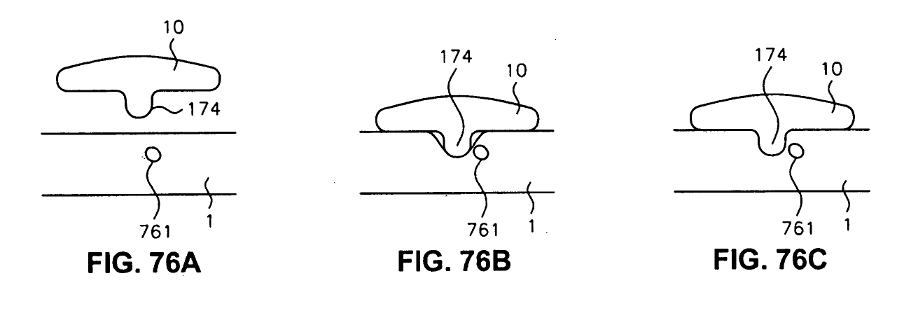

As shown in FIG. 7, main segment 10 can be positioned and/or stabilized adjacent the heart by stabilization protrusions 174, such as pegs, studs, and the like, including the stabilization protrusions described in FIGS. 76a-82b.

As shown in FIGS. 9A, main segment 10 can also include an extension segment 10a having an end 10b for attachment of a stabilizer/reconfiguration segment 12. End 10b can be removably connected to one or more means for positioning and/or stabilizing main segment 10 adjacent heart 1.

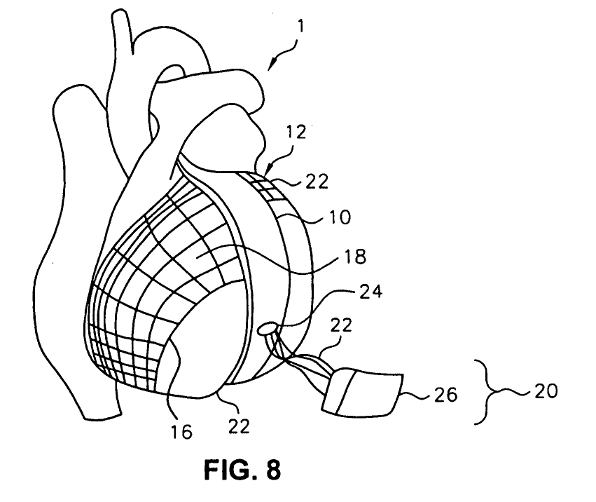

Stabilizer/reconfiguration segment 12 can also be adjusted to control position, stability, and/or support of the device, as shown in FIGS. 7, 10A, and 10B. FIG. 7 illustrates one embodiment of an adjustment mechanism 20 for adjusting and/or maintaining a desired shape and/or positioning of the main segment 10 and/or stabilizer/reconfiguration segment 12. Adjustment mechanism 20 shown in FIG. 7 includes a string/cable 22 which extends through main segment 10 and or through stabilizer/reconfiguration segment 12 as shown in FIG. 8. String/cable 22 extends out of the main segment 10 at an opening 24 and into an adjustment control mechanism 26 that adjusts the length of string/cable 22, thereby altering the position and/or size of stabilizer/reconfiguration segment 12 during or subsequent to placement.

FIG. 10B illustrates a stabilizer/reconfiguration segment 12 that is formed of stays 17 connected via string/cable 22 to main segment 10. As shown in FIGS. 11A and 11B, stabilizer/reconfiguration segment 12 can include one or more guides 25 extending through openings 23 of stays 17 and through main segment 10 as shown in FIG. 11B.

As shown in FIGS. 12A, 12B, 13A, 13B, 14A and 14B, main segment 10 can also be sized and shaped to support the base or other portions of one or more papillary muscles of either the mitral and/or tricuspid valve of heart 1. Main segment 10 can include, for example, a segment 10c for papillary support, integral with main segment 10, for supporting the base or other portions of one or more papillary muscles.

Embodiments of the stabilizer/reconfiguration segment 12 include:

(1) a totally flexible band or cord, approximately ‘C’ shaped, contoured to fit the outer surface of the left or right atrioventricular groove, that is fixed anteriorly and posteriorly to the members of an extracardiac remodeling clasp, as shown in FIGS. 7 and 8;

(2) a band or cord such as described in (1) above that has an extension intended to lie adjacent at least part of the external surface of the wall or the left and/or right ventricle and/or atrium as shown in FIGS. 7 and 8;

(3) a rigid collar or ring, approximately ‘C’ shaped, contoured to fit the outer surface of the left or right atrioventricular groove, that is fixed anteriorly and posteriorly to the members of an extracardiac remodeling clasp (FIG. 9);

(4) a rigid collar or ring, such as described in (3) above, that has an extension (10b) attachable to a stabilizer/reconfiguration segment 12 intended to lie adjacent at least part of the external surface of the wall or the left and/or right ventricle and/or atrium (FIG. 9);

(5) a ring, approximately ‘C’ shaped, contoured to fit the outer surface of the left or right atrioventricular groove, that is fixed anteriorly and posteriorly to the members of an extracardiac remodeling clasp including at least one main segment 10, of which some portion(s) is/are substantially flexible and other portion (s) is/are substantially rigid (as shown in FIG. 9);

(6) a rigid, flexible, or part-rigid, part-flexible ring, band, or collar, such as described in (1-5) above, of which the heart-contacting surface is a conforming cushion made of a fluid (e.g., gel) or ‘potting’ filled membrane sac;

(7) a rigid, flexible, or part-rigid, part-flexible ring, band, or collar, such as described in (1)-(5) above, of which the heart-contacting surface is a conforming cushion made of a soft solid polymer;

(8) a rigid collar or ring, such as described in (1)-(4) above, for which length can be adjusted in one or more dimensions by means of articulating, telescoping members (FIGS. 11A and 11B);

(9) a collar or ring, such as described in 8 above, for which telescoping members are controlled by traction via a sheathed string or cable (such as string/cable 22 shown in FIGS. 11A and 11B);

(10) a flexible cord or band, such as described in (1)-(9) above, for which length can be adjusted by traction on one or more enclosed cords or cables (such as cable 22 shown in FIGS. 11A and 11B; in a purse-string fashion in FIGS. 10A and 10B);

(11) a cord or band, such as described in (10) above, in which the enclosed cord or cable length is controlled by traction on sheathed extensions of the cord or cable;

(12) a part-rigid, part-flexible ring, such as described in (5) above, for which length may be adjusted by one or more of the mechanisms described in (8-10);

(13) a ring, collar, or band, such as described in (1)-(12) above, that is fixed to, and stabilized by, a flexible band that circumscribes at least part of the length of the opposite side of the heart (either in addition to or instead of stabilization by and fixation to the members of a heart-remodeling clasp);

(14) a ring, collar, or band, such as described in (1)-(12) above, that is fixed to and mutually stabilized by another ring, collar, or band that circumscribes the atrioventricular groove on the opposite side of the heart (either in addition to or instead of stabilization by and fixation to the members of a heart-remodeling clasp);

(15) one or more tabs extending to one side of a member of a ventricular remodeling clasp, or other framework on the heart, positioned to exert normal or tangential force on a region of ventricular wall that supports the base of a papillary muscle;

(16) an integral part of a ventricular remodeling clasp, or other framework on the heart, positioned to exert normal or tangential force on a region of ventricular wall that supports the base of a papillary muscle;

(17) one or more rigid ‘tabs’ that extend from or are optionally integral with a framework, such as a heart remodeling clasp, positioned to displace the ventricular wall segment that includes a papillary muscle base toward the heart base or the cavity (FIGS. 9, 12A, 12B, 13A, 13B, 14A, and 14B); and

(18) one or more areas of deviation that extend from a framework, such as a heart remodeling clasp, positioned to displace the ventricular wall segment that includes a papillary muscle base toward the heart base or the cavity (FIG. 12B).

Multi-Segmented, Self-Orienting, Heart-Contacting Plates for Heart Geometric Remodeling

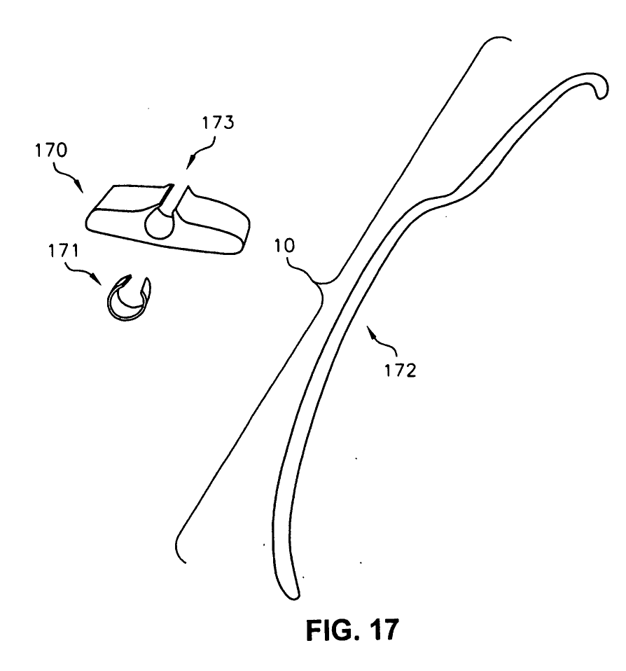

In one embodiment, and as illustrated in FIG. 17, the present invention also provides a heart-contacting main segment 10 that can be employed with the devices of the present invention. In one embodiment, main segment 10 includes one or more segment plates 170 that can be structured and mounted for rotation about the axis of a rigid frame 172. Rigid frame 172 maintains the centerline of plate 170 in the position prescribed to improve cardiac function (whether as part of a passive device, e.g., a restructuring assembly of the type disclosed herein or an active device, e.g., a wall-actuating assembly disclosed in U.S. Pat. No. 5,957,977, incorporated herein by reference. The permitted segmental or local axial rotation by the plates 170 and the balance of forces dictate that the most stable (lowest-energy) rotational position at any location is transverse tangentially to the heart surface. Segment rotation is sufficiently independent such that a plate 170 or part of a plate 170 may pivot if such a configuration is needed to maintain local tangent conformity to the surface of the natural heart.

A cross-section of the locally-rigid frame on which plates 170 are mounted can be, at least in part, arcuate or circular, and plates 170 can be mounted on the frame without axial fixation, such that the plates may rotate. By having very low torsional rigidity in the long axis of plates, different areas of plates 170 may rotate independent of each other. One advantage is that transverse (meaning perpendicular to the local long axis of the mounting frame) orientation of plates 170 adapts, because of the balance of moments imposed by reaction of the heart surface, to tangency with that surface resulting in substantial or full surface contact.

FIG. 17 also illustrates an embodiment of a plate 170, a plate spacer 171, and a frame, shown as rod 172, constructed in accordance with principles of one aspect of the present invention. In one embodiment, plate 170 illustrated in FIG. 17 has a slit or opening 173 adapted to accommodate plate spacer 171. Plate 170 can have any desired shape depending on the particular location of the natural heart or portion thereof to which it is to be applied. In one embodiment, plate 170 is convex in shape, where the convexity is toward a surface of the natural heart or portion thereof to which plate 170 is applied.

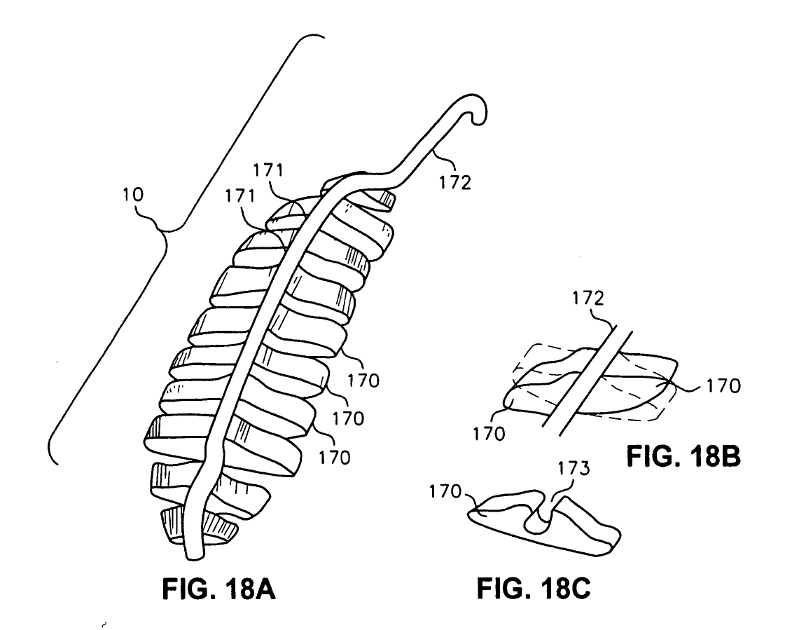

As shown in FIGS. 18A, 18B, 18C, one or more segment plates 170 can be positioned on rod 172. Segment plates 170 can include segment plate spacers 171 and can be attached to rod 172, for example, by a snapping action. In one embodiment, plates 170 can be fixedly attached to rod 172 such that the plates 170 do not pivot or rock with respect to rod 172. In a preferred embodiment, shown in FIG. 18B, plates 170 are removably attached to rod 172 such that plates 170 can pivot or rock and remain tangential with respect to the surface of natural heart 1. It should be appreciated by those of ordinary skill in the art that plate 170 can be attached to the frame by conventional means, such as by a ferrule coupling or pressure fitting, etc.

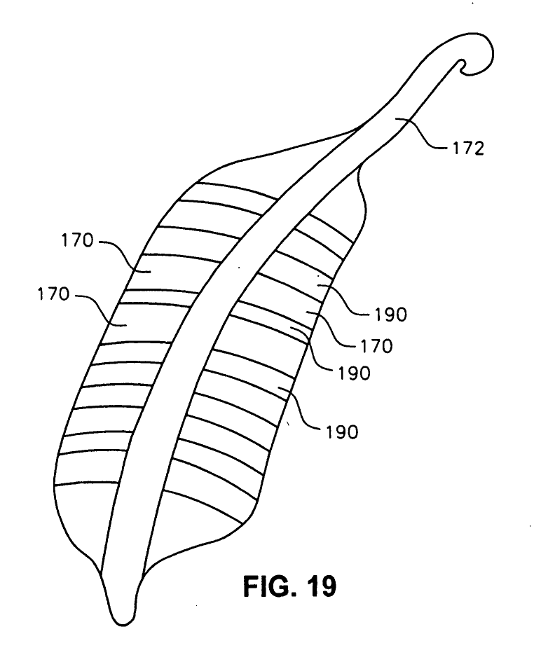

In another embodiment of the present invention, plate 170 can also be partially or fully covered by a shell 190, as illustrated in FIG. 19. The shell 190 serves to protect the patient against infection (e.g., by excluding tissue fluid from poorly-exchanged spaces where it would be a culture medium for bacteria) and also protects the heart surface against erosion by discontinuities between plate components. Preferably, shell 190 is composed of a biocompatible flexible, low-durometer polymer. In one embodiment, shell 190 includes a gel surrounding plates 170. In another embodiment, shell 190 is a solid shell formed of a uniform polymer material.

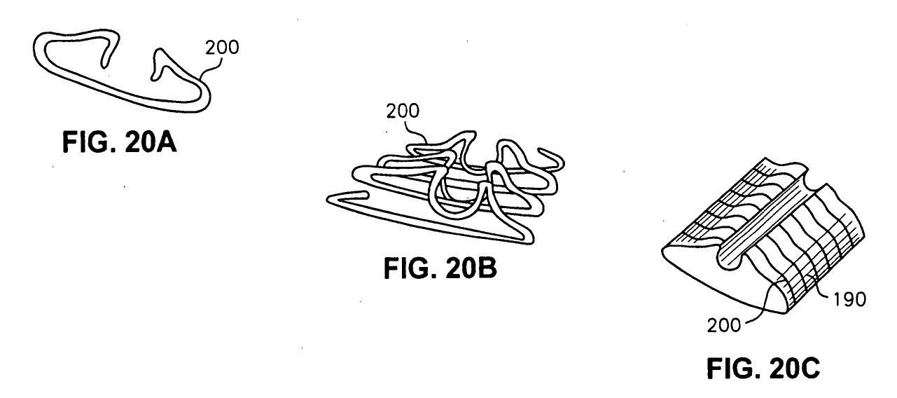

Plates 170 also can be formed from one or more plate wires 200, as shown in FIGS. 20A, 20B and 20C. In one embodiment of plate wire 200, illustrated in FIG. 20A, includes a series of single wires. In another embodiment, illustrated in FIG. 20B, plate wire 200 includes a continuous spiraled wire. As shown in FIG. 20C, plate wire 200 can be contained within shell 190.

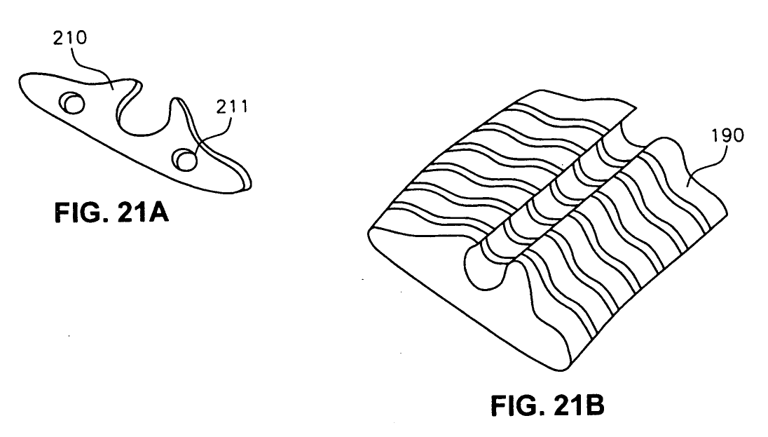

Another embodiment of the heart-contacting plate used for a main segment 10 according to the present invention is illustrated in FIGS. 21A and 21B. As shown in FIG. 21A, the heart-contacting plate can include a rigid or semi rigid plate 210. Plate 210 can include an opening 211 to accommodate the flow of the material forming shell 190 through opening 211 such that the rigid segment plate is embedded within shell 190, as shown in FIG. 21B.

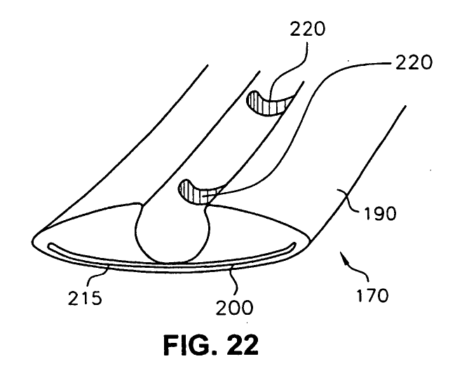

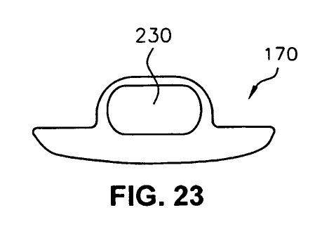

Another embodiment of a heart-contacting plate according to the present invention is illustrated in FIG. 22, main segment 10 is formed from individual plate wires 215 embedded in a soft, elastomeric encapsulating material of shell 190. In one embodiment, segment plate wire 215 and shell 190 can have a convex surface that contact the heart, such as that illustrated in FIG. 22. FIG. 22 also illustrates holes 220 which allow the passage of stabilization protrusions 174 such as pegs shown in FIGS. 7, and 76A-82B, through shell 190 into heart 1. This aspect is discussed in more detail below. FIG. 23 illustrates a cross-section of another embodiment of a heart-contacting plate 170 of the present invention in which a plate 170 includes an opening 230 (e.g., a round or oval opening) through which rod 172 can pass.

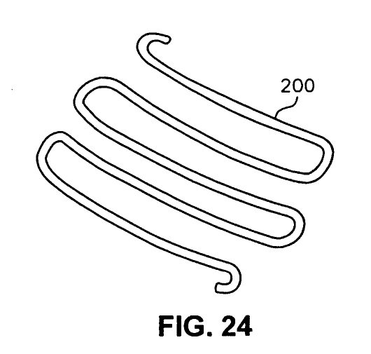

Plate wire 200 can also have a flat zigzag configuration, as shown in FIG. 24, prior to encapsulation in shell 190. In this zigzag configuration, adjacent segment plate wires 200, optionally, can be joined by a bend in the wire at each wire end. In one embodiment, adjacent plate wires 200 are formed from a continuous wire.

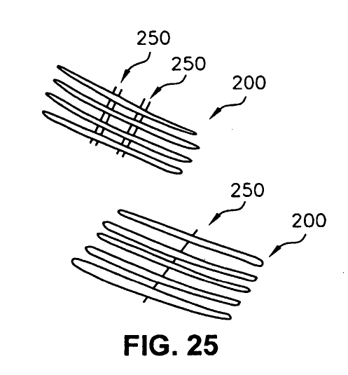

FIG. 25 illustrates an embodiment in which plate wires 200 are connected by one or more plate wire connectors 250. Plate wire connector 250 is preferably mounted substantially perpendicular to the plate wires 200. Plate wire connector 250 can include, for example, a polymer or wire attached to each plate wire 200, for example, by welding, soldering, or the use of an adhesive. The purpose of wire connector 250 is to facilitate placement of plate wires 200 or similar elements into a mold, and stabilize their position during application or injection of the low durometer polymer or other suitable material to form shell 190.

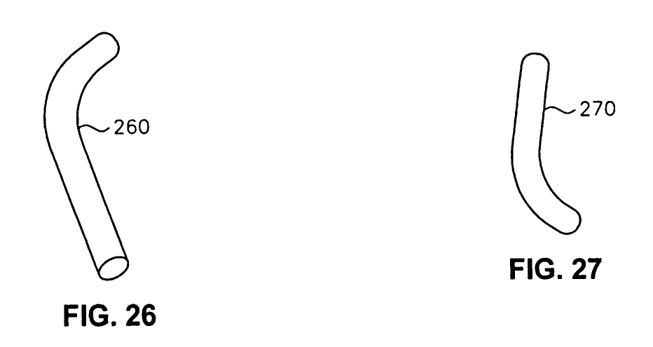

Main segment 10 of the present invention can include a single frame piece or individual components connected together to form main segment 10. In one embodiment, illustrated in FIGS. 26-28, main segment 10 comprises an apical segment (270), atrial segment (260), and main segment (10), all of which are sized for the particular dimensions of heart 1. Atrial segment 260, as illustrated in FIG. 26, can be configured for placement adjacent the atrial wall. As shown in FIG. 27, apical segment 270 can be configured for placement adjacent the ventricular apical wall. The outer surfaces of atrial and apical segments 260 and 270 shown in FIGS. 26 and 27 can be covered by a textured material, such as, for example, a velour, porous (such as a mesh) fabric, to facilitate tissue ingrowth and fixation.

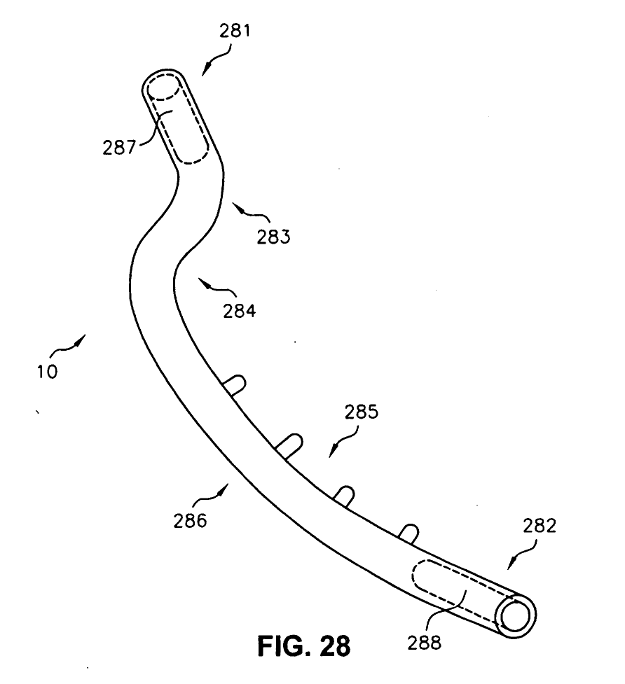

FIG. 28 illustrates an embodiment of a main segment 10 having a central spine 286 that is configured for placement adjacent a portion of the ventricular wall and atrioventricular junction and extensions 281 and 282 that are either straight or arcuate, depending on the shape of heart 1. More specifically, main segment 10 illustrated in FIG. 28 includes extension 281 having a connector portion 287 (such as a hollow section for releasably accommodating atrial segment 260, as shown in FIG. 26) for connection to apical segment 260; a curved section 283 convex to the heart, approximating a circular arc of about 60 to 90 degrees and intended to lie adjacent the atrioventricular junction, preferably having a radius of curvature ranging from about 5 to about 15 mm; a ventricular shoulder section 284 concave toward the heart, having a circular arc, generally having a radius of curvature of about 10 to about 30 mm, and generally extending about 60 to about 90 degrees; a main section 285 that is approximated by a circular arc (for example, having a radius of curvature of about at least about 100 mm or greater) or an elliptical arc (having a major hemi-axis of at least about 100 mm or greater); and a connector 288 (such as a hollow section for releasably accommodating apical segment 270 as shown in FIG. 27) for connection to apical segment 260.

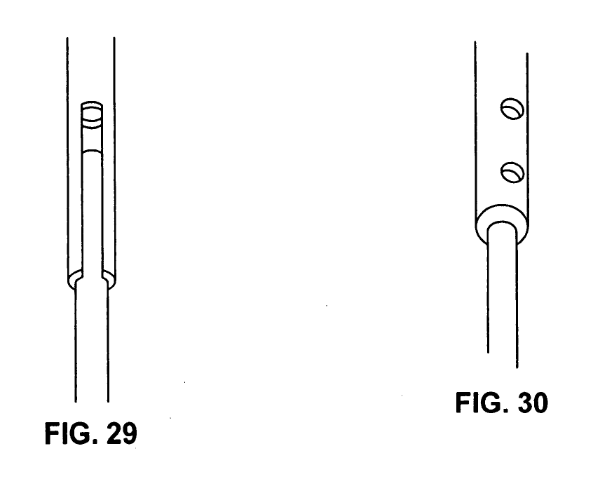

FIGS. 29 and 30 illustrate embodiments of extensions 281 and 282 for connection to the atrial segment 270 and apical segment 260, respectively. In a preferred embodiment, extensions 281 and 282 are telescoping and include indexed (e.g., ball and socket or ratchet) or continual sliding adjustment mechanisms. Alternatively, extensions 281 and 282 can be side-by-side interlocking grooves that provide flexural stability. Extensions 281 and 282 may be circular or non-circular in cross-section. Straight extensions are preferred, as the degree of telescoping does not impose any change in the relative angulation of the two ends of the complete rod assembly. If extensions 281 and 282 are curved, the degree of combined (between both atrial segment 260 and main segment 10, and between apical segment 270 and main segment 10) telescoping without unacceptable change of end angulation may be limited.

Generally, closed, non-communicating spaces that would contain stagnant tissue fluid should be avoided. This can be accomplished, for example, by open-sided, outside telescoping section as shown in FIG. 29, or by one or more fenestrations in the outside telescoping section, as shown in FIG. 30. It should be appreciated that conventional means of position locking after adjustment of the length of rod 172 can be employed, including, but not limited to, set screws and tightening collets (e.g., a metal band, collar, ferrule, or flange).

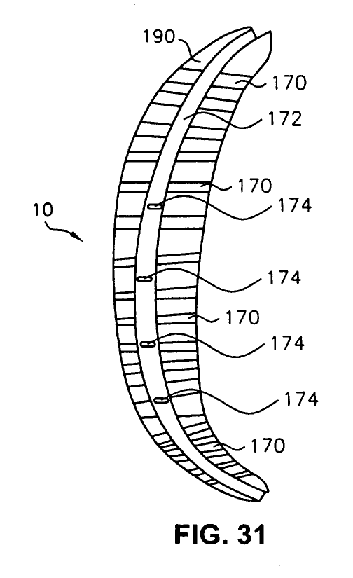

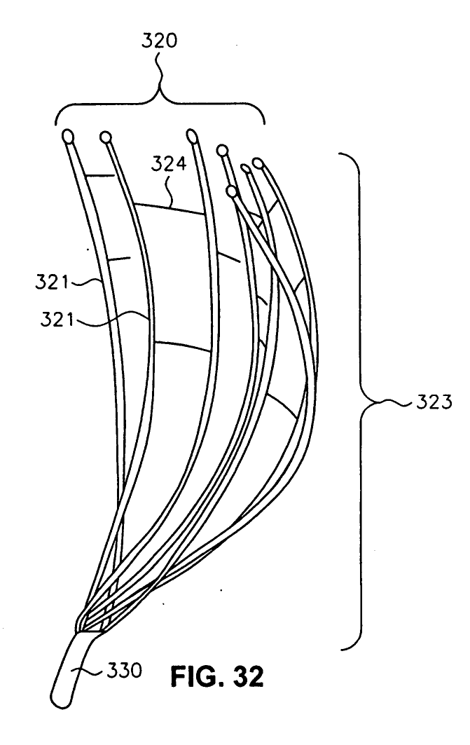

FIG. 31 illustrates main segment 10 including a multiple of plates 170 (not shown), rod 172 and shell 190 forming heart-contacting surface 27 of a pliable and/or elastic material for placement adjacent the ventricle or a portion thereof, the atrio-ventricular junction, and part or all of the atrial wall, preferably the portion of the anterior or posterior atrial wall nearest the atrioventricular junction. Plate 170 and rod 172 can be pre-attached, either flexibly or rigidly, or can be joined at the time of placement of the device. Multiple plates 170 attached to a single rod 172 can be formed according to any of the embodiments shown in FIGS. 17-28.

The present invention also includes an embodiment where a single large plate (e.g., a solid, semi-solid, fluid or ‘potting’ filled pad) in the shape as shown in FIG. 31, is substituted for a multiple of plates 170. The single large plate or pad is attached to rod 172 and has sufficient torsional flexibility over its entire length such that the plate can conform to a surface of the natural heart to which it is to be applied and maintain a position substantially tangent to the natural heart surface even while the heart contracts and expands.

The mounting framework for a heart remodeling device according to the present invention that employs plates 170 or a large single plate, of the present invention is made of a generally circular or round cross-section rod 172. Rod 172 is curved so that its inner (toward the heart) surface approximates the centerline of intended heart-wall contact. Mounted on this framework are an alternating series of plates 170, alternating with plate spacers 171. Plates 170 are approximately rectangular when viewed from the direction of the heart surface. When viewed from a direction along the local frame axis, the heart-contacting surface is generally a circular arc, having a radius of about 60 to 200 mm, or an elliptical arc (having a major hemi-axis of at least about 100 mm or greater). On the opposite side, viewed from this same direction, there is a notch of a width and shape to accept and snap onto rod 172, after which plate 170 may rotate on rod 172. Spacers 171 are part of a circle, that similarly fit onto rod 172, alternating with plates 170.

Plates 170 are generally about 1 to 12 mm in the dimension that parallels the local orientation of the frame. In that same dimension, spacers 171 are generally about 1 to 12 mm. Plates 170 are generally about 12 to 30 mm in the direction that is both perpendicular to the local frame and parallel to the local heart surface, the width intended for the completed frame at that location. This dimension, as well as the radius of curvature for the plate 170 surface that is to contact the heart, is computed from heart diameter, wall thickness, geometric values, and the intended epicardial to cavitary pressure ratio and extent of intended radius reduction.

In the direction that is perpendicular both to the local frame and to the local heart surface, the dimension of rod 172 and plate 170 is sufficient to effect sufficient flexural rigidity across the width of the completed plate 170 to prevent substantial deformation under expected forces when mounted on rod 172 and used to deform the heart as intended clinically. After assembly, the entire plate 170, spacer 171 (if used), rod 172 assembly is covered with a low durometer polymer, that is biocompatible, such as a polyurethane or a silicone rubber, as in FIGS. 20c and 31.

The present invention reduces or eliminates non-tangential contact between plates of a ventricular geometric remodeling device and the ventricular epicardial surface. Consequences of such non-tangential contact are mediated by excessive pressure, and include local subepicardial tissue ischemia, coronary artery occlusion and/or damage, and possible erosion into the surface. The present invention also reduces or eliminates the attendant risk of excessive localized pressure which may cause one or more of the above consequences.

Plates 170 are different from standard plates 550 (such as that shown in FIG. 55 below) that are fixed to the support structure of a remodeling device (such as a heart remodeling device such as the CardioClasp) in that the plates 170, upon contact with the epicardium, rotate to the lowest-energy (most stable) position, preferably tangent to a surface of heart 1.

An advantage of the invention is that the lowest-energy (most stable) position, because of the structure of plate 170 mounting, is tangent with the epicardium, rather than a fixed orientation to the frame of the device, which would risk edge effects and excessive contact pressure between the remodeling device and heart 1.

Variations of the invention include:

(A) An assembly similar to that shown in FIGS. 19, 20C, 21B, 22 and 31 (all of which may include a low-durometer polymeric filling, ‘potting’, or fluid such as a gel), may also be used but without the low-durometer polymeric filling, ‘potting’, or fluid (e.g., a gel).

(B) Plates 170 (such as in FIG. 17) made of a low-durometer polymer, such as a polyurethane or a silicone rubber, that is reinforced by embedded wire, either a multitude of wire loops or links of coiled wire. In one embodiment, the wire reinforcement provides sufficient rigidity of the surface in the direction perpendicular to the long axis of plate 170. Plates 170 themselves have little torsional rigidity or intrinsic longitudinal rigidity. Longitudinal rigidity is imposed, however, by cylindrical rod 172 onto which plates 170 are mounted. Mounting may be either via a central hole or bore through the long axis of plates 170 or (preferred) a slot in the surface (the ‘free surface’) opposite that contacting the heart. The width of the slot decreases, at least at intervals, to slightly less than the diameter of rod 172 near the free plate surface so as to allow a ‘snapping-on’ type of position stability. As is the case with plates 170 (either (A) above or the preferred embodiment described earlier), blunt stabilization protrusions 174 or fixation pegs, if used, would be mounted in or to rod 172 and pass through holes in heart-contacting surface 27 of shell 190.

(C) Plates meeting the description of (B) except that the reinforcement plates or wires are multi-perforated, generally 1 to 3 mm thick, mini-segments of rigid biocompatible polymer or metal embedded at intervals in the of the low-durometer polymer shell 190. The mini-segments impose and permit the same range of rigidity as do the wire reinforcements of (B).

Systolic to Diastolic Pressure Transfer Mechanism

In FIGS. 32-45, there are shown a number of embodiments of heart assist and reconfiguration devices including elastic members placed inside or outside the heart and configured to contact a portion of a heart wall to exert a force thereon. As shown, these embodiments generally comprise one or more spring members configured to be positioned adjacent a section of a heart wall and to be biased against the heart wall. This may be accomplished by various configurations of wire leaf spring members.

Alternatively, this may be accomplished by suitably shaped and heat treated metal such as stainless steel or shape memory metal such as nitinol, forming a suitably configured shell, possibly configured by computerized conformation to the shape of the desired location within or outside the heart, and then laser etching the device from the shell.

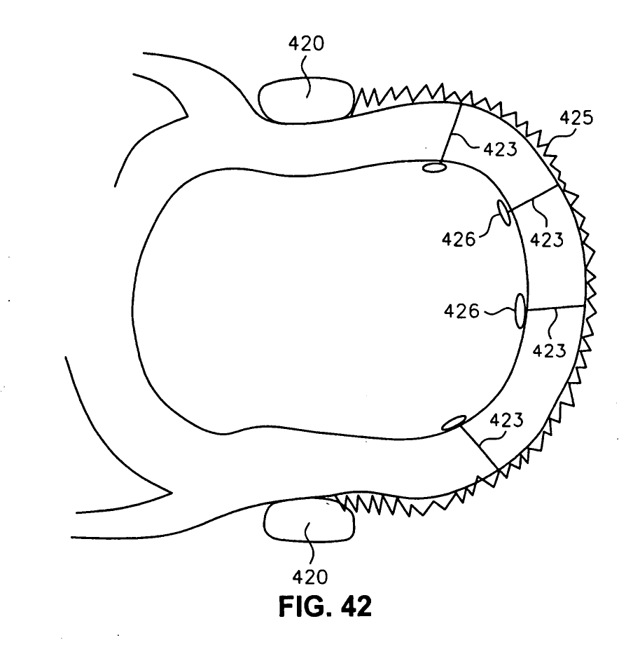

As shown in FIGS. 32-45, these embodiments include a spring mechanism including a fan-like array 323 or a single spring element such as spring 425 in FIG. 42, that exerts outward force against the inside of the left or right ventricle of the heart. The spring mechanism works by storing energy while the ventricular walls move centrally during active contraction of the ventricles (cardiac systole), and releases that energy while the ventricular walls move outward during passive relaxation of the ventricles (cardiac diastole). By using preferably metallic (such as CP titanium or stainless steel) springs, with low hysteresis or energy loss, relatively little energy is lost. Since the movement of the ventricular walls in contraction and in relaxation is equal and opposite, near-equality in energy storage and release means that the pressure effect will be the same. That is, the spring mechanism will reduce pressure within the ventricle by a numerically near-equal amount in systole and in diastole, at equivalent ventricular size. The pressure decrement will be the same in early systole as in late diastole, in mid-systole as in mid-diastole, and in late systole as in early diastole. When the wall moves inward with contraction, spring mechanism is also deformed inward. This exerts an outward force on the wall both during contraction and relaxation that is determined principally by the instantaneous ventricular circumference. The relationship between instantaneous circumference and pressure decrement is dependent on the characteristics of the spring mechanism, such as the effective spring constant if its structure renders it linear in action, its tangent spring constant at each level of deformation otherwise, and its resting configuration. The natural outward force of the ventricle, simultaneous size and shape of the ventricle as well as the spring constant determine the absolute amount of pressure decrement, that is, the difference in chamber pressure from what it would be if the spring mechanism were absent.

The spring mechanism can be used for patients who have symptoms or risks associated with decreased compliance of the ventricles during filling. This is generally manifested by increased pressure in the ventricle(s) at the end of filling (elevated left or right ventricular end-diastolic pressure, LVEDP or RVEDP), which in turn leads to elevated left or right atrial pressure and then to elevated pressure in the veins draining the lungs (pulmonary veins) or the veins draining the body (systemic veins), respectively. Symptoms of a left sided problem include shortness of breath and risks are dangerously low oxygen saturation because of fluid in the lungs (pulmonary congestion, progressing to pulmonary edema). Symptoms of a right sided problems include swelling of the legs and feet, followed by, fluid in the abdomen and swelling of abdominal organs, particularly the liver, while risks are poorer blood flow through organs, particularly the liver, and failure of those organs.

The spring mechanism is also suitable to provide a margin of reserve in the strength of contraction of the ventricles such that reduction of the systolic (contracting) pressure in that ventricle or ventricles would be expected to cause lesser problems than those relieved by reducing the diastolic (filling) pressure of that same ventricle.

Accordingly, the spring mechanism is useful in, but not limited to, such patients as recipients of a treatment, such as geometric remodeling of a ventricle with or without a specialized device as described herein or in U.S. Pat. No. 5,702,343 (Acorn), U.S. Pat. No. 6,085,754 (Acorn), U.S. Pat. No. 5,961,550 (Myocor) or U.S. Pat. No. 5,800,528 (Abiomed) all of which are hereby incorporated by reference, or recipients of a partial left ventriculectomy. One advantage is a well tolerated partial loss of now-excessive systolic pressure reserve in exchange for a significantly beneficial reduction of diastolic filling pressure. These treatments may tend to induce an upward (which would be unfavorable) proportional change in ventricular filling pressure that is, relative to the basal filling pressure, similar to the favorable proportional upward change in ventricular ejection pressure reserve. However, since baseline ejection pressures are from 4 to 15 times as high as baseline filling pressures, similar arithmetic reduction in each will have a much more significant favorable effect on filling pressure than it does an unfavorable effect on ejection pressure.

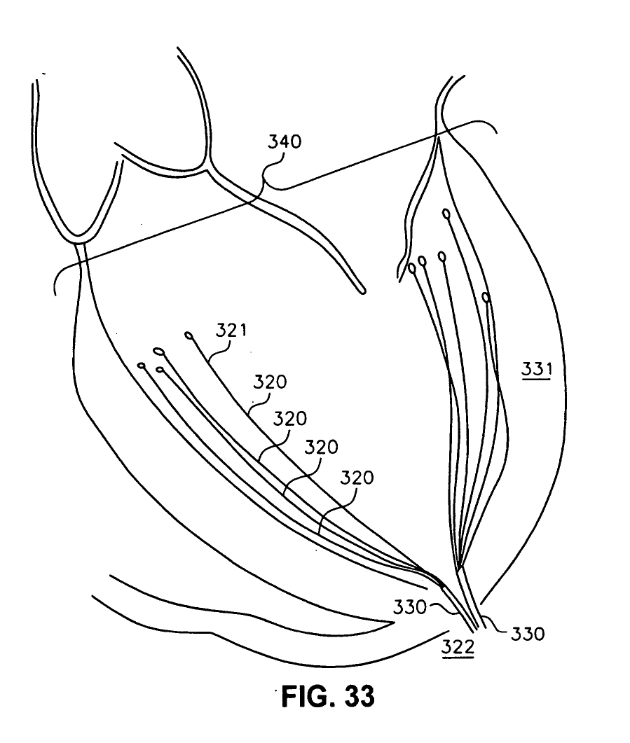

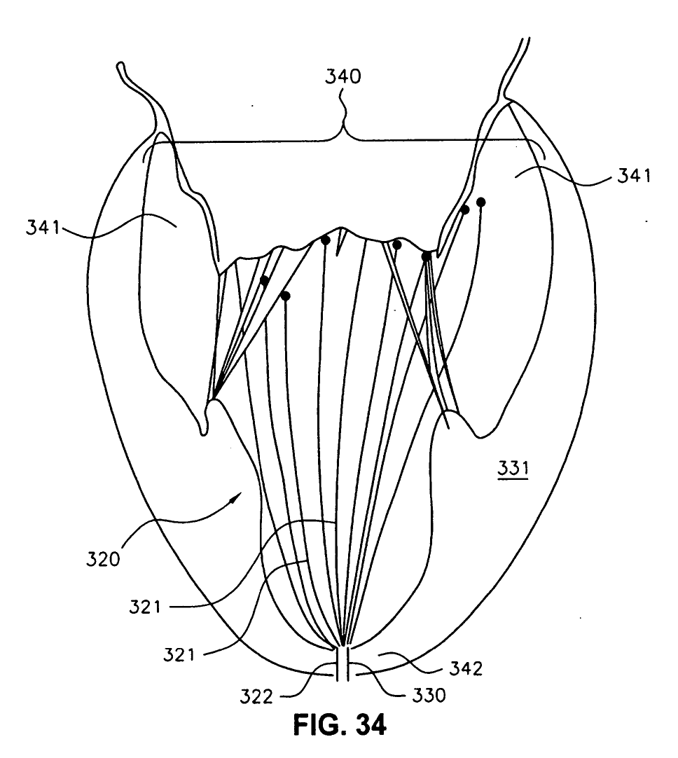

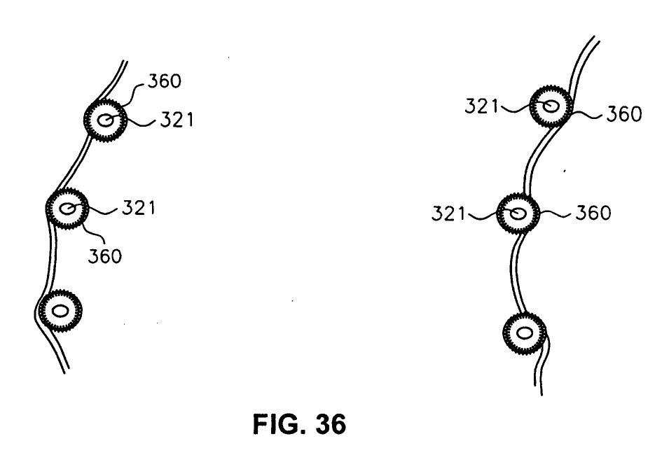

In one embodiment, the spring mechanism includes at least one, preferably two, and possibly more than two, bundles 320 of spring wires 321 that lie against the inner walls of the ventricle, as shown in FIG. 32. Spring wires 321 of each bundle 320 or a plurality of bundles 320 are fixed to each other at one end 322, placed at or near an apical end of the ventricle. From that point, each bundle forms a fan-like spring array 323 with each wire 321 extending toward the base 340 of the ventricle as shown in FIGS. 33, 34, and 37B. Spring wires 321 may, or may not, be individually covered by a porous or textured polymer covering 360 (as shown in FIG. 36), such as expanded polytetraflurethelene (ePTFE). Similarly, wires 321 of a bundle 320 may be joined by polymer strands or tethers 324.

The set curvature of individual wires 321, and their alignment at the point of joining, is such that when released, the array of wires 321 in a bundle 320 conforms to part of a hollow solid somewhat larger than the ventricle being remodeled. In the case of the left ventricle, this would be in the general shape of part of an ellipsoid of revolution of minor axis greater than that of the ventricle. Both the resting shape of spring wires 321 and the flexural rigidity of spring wires 321 are selected such that an average outward force is exerted on the ventricle at all points in the cardiac cycle commensurate with the desired reduction in cavitary pressure. At the point of junction, such as post tip 330 of spring wires 321 of each bundle 320, spring wires 321 coalesce into a solid rod, fabricated by welding or by adhering with a biocompatible adhesive two or more spring wires 321, such as by using an epoxy compound.

The present invention embodied in FIGS. 32-45 treats the problem of symptomatic or hazardous elevation of diastolic pressure in the cardiac ventricle(s). It is different from either vasodilating or diuretic medications in that there is no reason to expect any effects other than on the heart. In addition, there is no direct risk of renal (kidney) damage or dysfunction, of electrolyte imbalance, or of dehydration using the present invention, in contrast to the use of medicines. Furthermore, there is a lesser risk of symptomatic hypotension using the diuretic present invention than with the use of vasodilator medicines.

FIG. 32 illustrates one embodiment of the present invention. As shown in this figure, bundles 320 of spring wires 321 can be composed of spring wires 321 having an apical end 322 and linked by interlinking strands or tethers 324.

FIG. 33 illustrates halves of two bundles 320 shown inside and against the wall of a longitudinally sectioned left ventricle 331 (cut perpendicular to septum, viewing toward posterior wall) and having post tips 330.

FIG. 34 illustrates bundle 320 shown as seen from inside a longitudinally sectioned left ventricle (cut parallel to septum, viewing toward free wall 341), in relation to the apex 342 of the ventricle and base 340 of the ventricle.

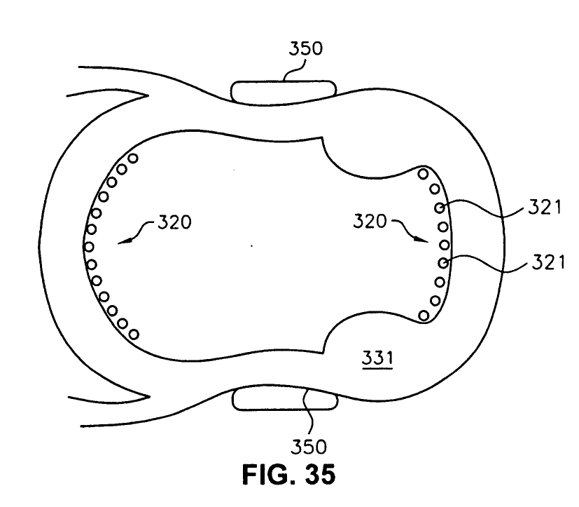

FIG. 35 illustrates a top view of a transverse section of a heart in which two bundles 320 have been positioned against the free wall and septum, respectively, of the left ventricle. FIG. 35 illustrates bars or plates 350 of a ventricular remodeling device (as shown, for example, in FIGS. 10A and 10B) which may be used in conjunction with a spring mechanism or another heart remodeling or surgical procedure such as those known to the art, including U.S. Pat. No. 5,702,343 (Acorn), U.S. Pat. No. 6,085,754 (Acorn), U.S. Pat. No. 5,961,550 (Myocor), U.S. Pat. No. 5,800,528 (Abiomed), or those described in McCarthy et al., “Early results with Partial Left Ventriculectomy,” from the Departments of Thoracic and Cardiovascular Surgery, Cardiology and Transplant Center, Cleveland Clinic Foundation, Presented at 77^{th} Annual Meeting of the American Association of Thoracic Surgeons, May 1997, 33 pages, all of which are hereby incorporated by reference.

FIG. 36 illustrates an enlarged view of the illustration in FIG. 35. As shown in FIG. 36, spring wires 321 can be covered with a polymer covering 360, such as a polymer such as knitted polyester, to facilitate tissue ingrowth. FIG. 36 illustrate cross-sections of such covered spring wires 321, respectively, before (left-side) and after (right-side) tissue ingrowth surrounding spring wires 321.

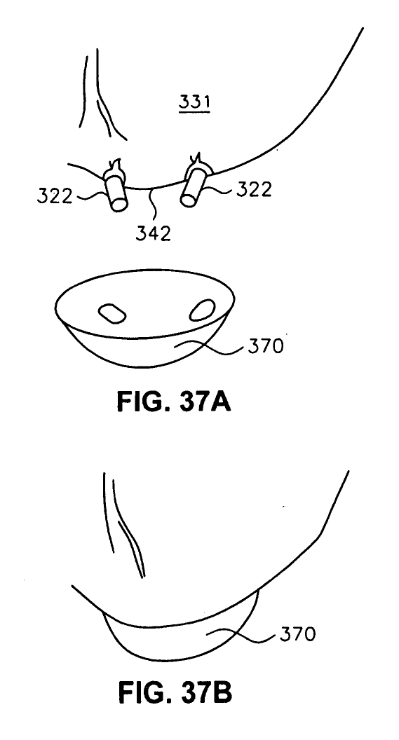

FIG. 37A illustrates an embodiment of an apical stabilization coupling 370, such as an apical cap including a mounting block that rests adjacent the apical portion of the heart and stabilizes fan-like array 323 adjacent or within an apicandial surface of the heart. In one embodiment, coupling 370 also fixes two or more bundles 320 of spring wires 321 together at apical end 322. Ventricle 331 shown in FIG. 37A has not been subjected to a geometric remodeling device.

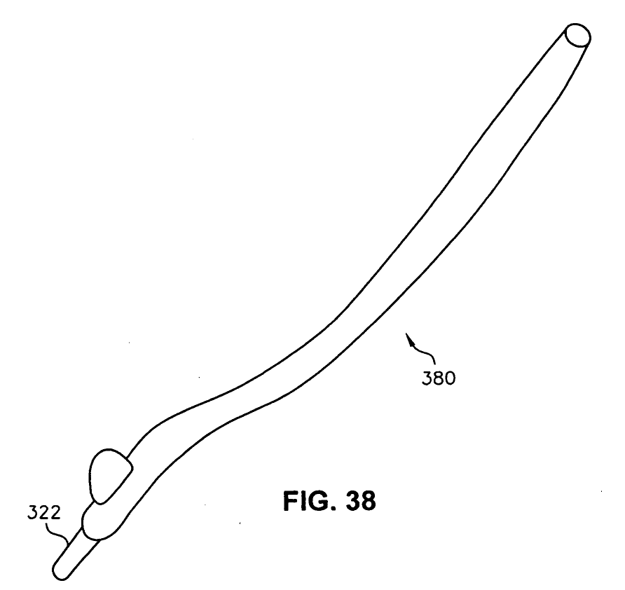

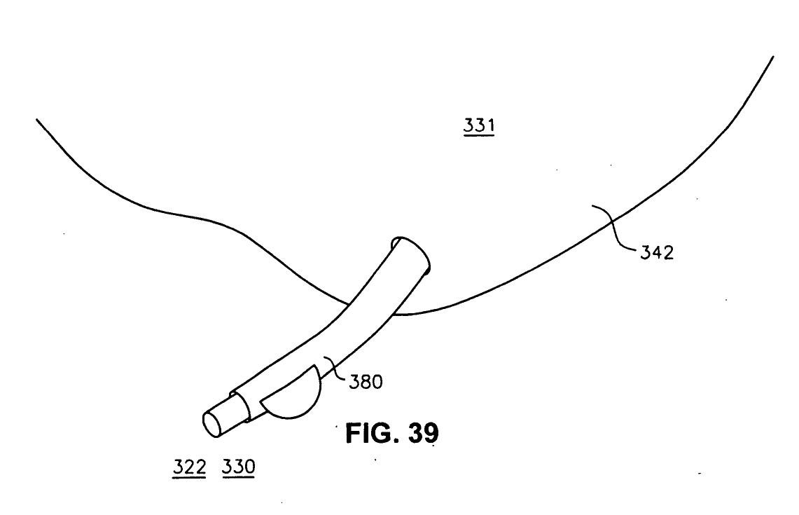

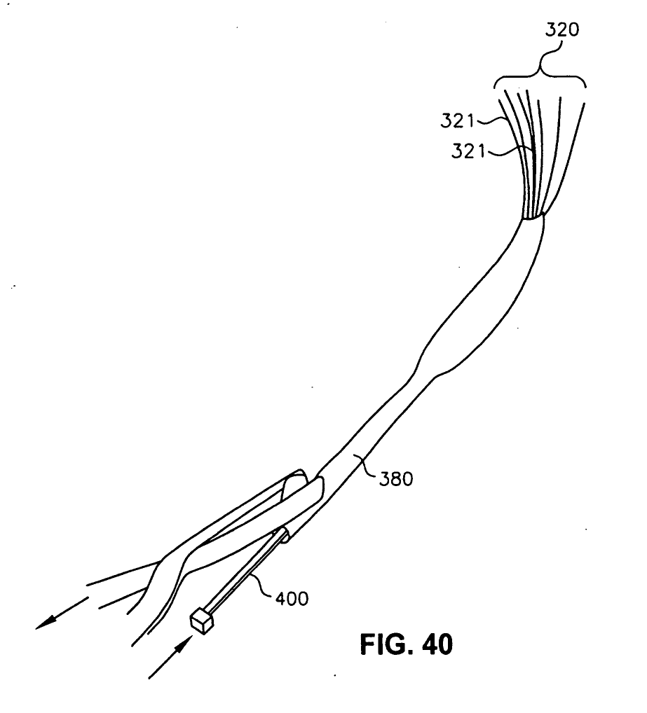

One method of positioning in a heart bundle 320 of wires 321 is shown in FIGS. 38-40. As shown in FIG. 38, the bundle 320 of wires 321 can be loaded inside a removable insertion sheath 380. Sheath 380, as shown in FIGS. 38 and 39, can then be inserted, for example, through an apical end of the ventricle. After insertion through the apical end of the ventricle, the removable insertion sheath 380 can be removed, for example, by traction and insertion of a stylus 400, as shown in FIG. 40.

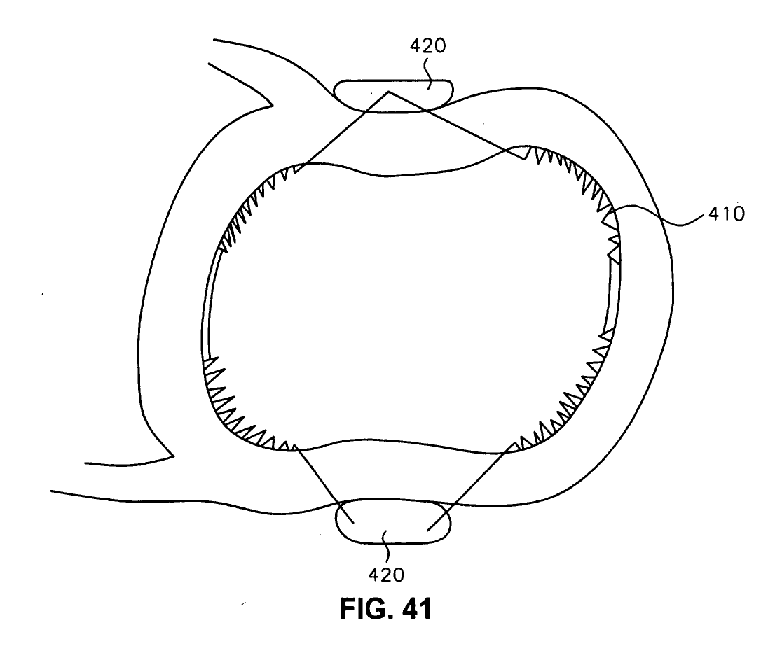

Another embodiment of the present invention is illustrated in FIG. 41. This embodiment includes one or more sections of helical, coiled or corrugated metallic spring wire 410 (referred to as a spring mechanism extending from the anterior to the posterior bar or plate 420 (such as a main segment 10 described herein) of a bimeridianal restraint type of ventricular geometric remodeling device. Spring wire 410 may be of one or more independent wire spring segments without inter-connection or contact, or they may be connected or interwoven during or before placement, or a continual spring segment. In one embodiment, each spring segment is connected at one end to one of the bars or plates 420 on the outside of the anterior wall of a ventricle, passing through that wall, crossing the inner (endocardial) surface of the interventricular septum and/or of the free ventricular wall, and passing through the posterior ventricular wall on its way to connection with another of these bars or plates 420 on the outer posterior wall. The ends of the spring mechanism are anchored to bars or plates 420, which exert force on the assemblies' opposite ends, compressing the ends toward each other, causing the center portion to exert outward force on the heart wall section that is traversed.

FIG. 42 illustrates another embodiment of the invention. In this embodiment the spring mechanism includes a spring assembly 425 anchored to remodeling plates 420 (of a bimeridianal restraint type of ventricular geometric remodeling device) on either end and extending across the outer (epicardial) surface of the ventricular free wall from one to the other of bars or plates 420. At intervals along spring assembly 425, struts 423 (e.g., pins, sutures, cords, cables, etc.) extend through the wall to buttresses 426 on the inner (endocardial) surface, segmentally tethering the spring assembly 425 to the wall so that when the wall moves inward with contraction, spring assembly 425 is also deformed inward.

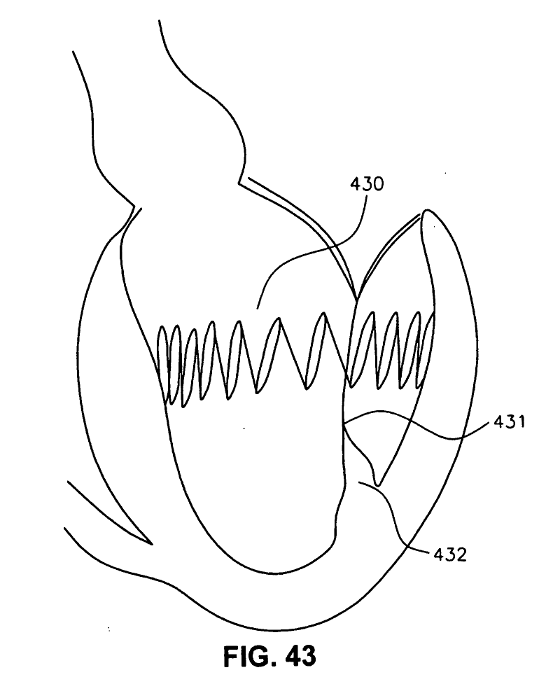

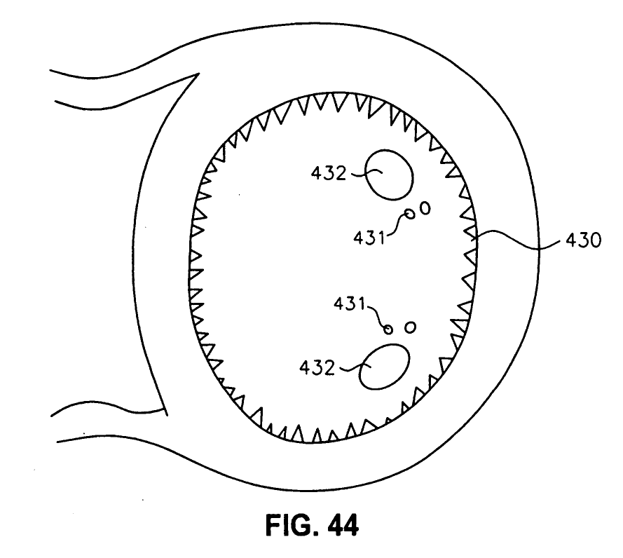

Another embodiment of the present invention is illustrated in FIGS. 43 and 44. In this embodiment, one or more spring mechanisms including spring assemblies 430 can be introduced into the ventricular cavity by one or more transvascular catheters, and assembled, by manipulation via the placing, for example, of catheters under fluoroscopic and/or echocardiographic visualization and guidance, into an encircling spring assembly 430 on the inner surface of the ventricle, lying on the inner surface of the ventricle at or near its largest circumference, between that inner (endocardial) surface and the valve-support apparatus (chordae tendinae 431 and papillary muscle tips 432).

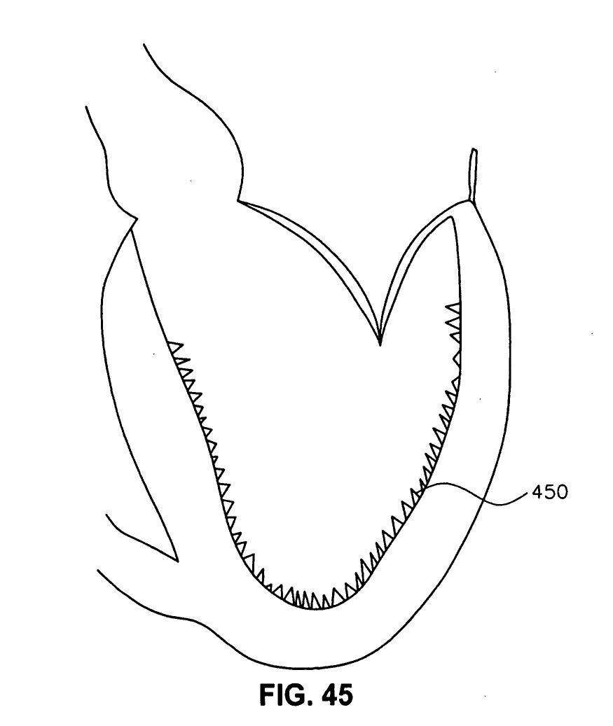

FIG. 45 illustrates a spring mechanism including a U-shaped spring assembly 450 that can be placed in the ventricle via a transvascular catheter under fluoroscopic and/or echocardiographic control, with attention to orientation and length of the arms of the ‘U’ so as avoid deformation and immobilization of the atrioventricular (mitral or tricuspid) valve of the ventricle. The center segment of the ‘U’ shaped spring assembly 450 can be positioned against the inner surface of the apical portion of the ventricle, while the two arms can be positioned against the interventricular septum and the free wall.

Spring assemblies 410, 425, 430 or 450 can also include two or more of the assemblies pre-attached to each other at the ventricular end that are separated upon release following transapical introduction into the ventricular cavity. Spring assembly 410, 425, 430, or 450 can also allow for adjustment of spring mechanisms after placement to alter the outward force/deformation relationship. This may be, but is not limited to, local deformation of one or more spring segments by traction or torsion via a transvascular catheter.

Method For Use

One embodiment of the method of use of devices according to the present invention includes the following steps. First, referring to FIG. 38, each bundle 320 of spring wires 321 is loaded into a separate removable, generally tubular, polymer sheath. A stab wound is then made in the apical end of the ventricle and dilated mechanically, with local pressure to control bleeding. The wire-containing sheath 380 is next introduced, with direction controlled by manual or instrument grasp of the solid post tip 330 of bundle 320. During guiding of the sheathed bundle into the ventricle, position is maintained with the basal end against the inside wall, so as to be generally between the wall and chordae tendinae and/or valve leaflets. When fully advanced, a stylus is inserted in the outside end of sheath 380 and post tip 330 is maintained stationary while sheath 380 is withdrawn. This releases wires 321 of bundle 320 to ‘fan-out’ against the inside (endocardial) surface of the ventricular wall. In a preferred embodiment, placement will generally be either against the lateral wall, between the papillary muscles, or against the interventricular septum.

When the desired number of bundle(s) 320 have been placed, the ventricular apical stab wounds are controlled by purse-string sutures or other mechanical means, with post-tips 330 protruding. Apical stabilization coupling 370 is attached to one or more post-tips 330, so as to control the position of bundles 320 relative to each other (where more than one bundle is used) and to the ventricular wall. In the event of concomitant placement of a ventricular geometric-remodeling device, such as a clasp described herein, post-tips 330 of spring bundles 320 may, or may not, be fixed to the apical components of the clasp, if any. The apical stabilization coupling may or may not be adjustable as to separation and relative angulation of post-tips 330.

Fluoroscopy is generally expected to be used during placement, with exposure of the cardiac apex either through a small open incision (intercostal or subcostal) or through a thoracoscope port.

The spring mechanisms described above can be made of biocompatible metals such as stainless steel and shape memory metals such as nitinol.

Tethered-Bar (O-Cable Clasp) Device for Bimeridianal Cardiac Geometric Remodeling

As discussed above with reference to FIG. 42, for example, tie present invention also provides a heart-remodeling device comprised of two rigid main segments 10, designed to be placed in contact with substantially opposite surfaces of a heart chamber, or of two contiguous heart chambers (such as the left ventricle and left atrium), and held to no more than a desired distance from each other by tethers (such as bands, cords, cables, chains, and the like) joining main segments 10 at their extremities, passing on the outside surface of the cardiac chambers. Such devices are sometimes referred to herein as a clasp or heart remodeling clasp or device.

These devices work by pressing inward on the walls of one or more chambers surrounded thereby, altering shape of the chamber or chambers. In doing so, the ratio of wall tensile stress to chamber pressure is reduced.

In common with other variants of bimeridianal restraint wall stress reduction devices, and in contrast to other heart-failure treatments, by reducing the ratio of wall stress to chamber pressure, this device provides the benefit of more effective heart muscle cell contraction that is mediated by cellular afterload reduction, but without the risk of excessive blood pressure lowering.

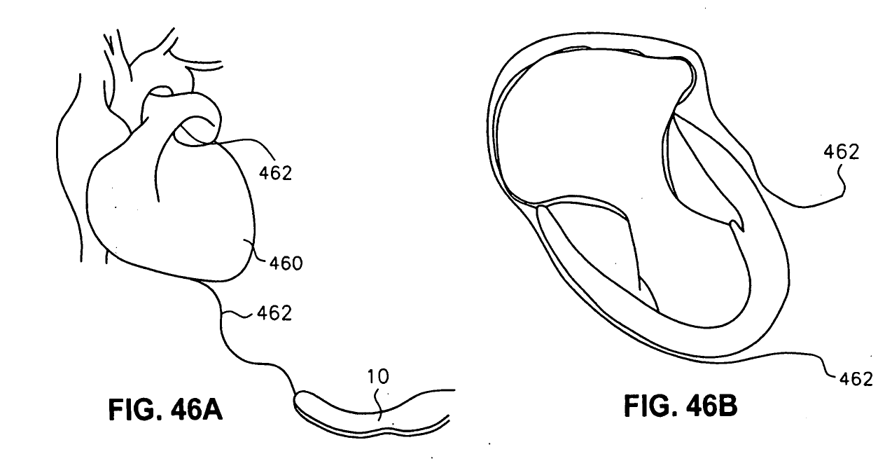

In contrast with other known variants of bimeridianal restraint devices, in most embodiments described herein, spontaneous ventricular torsion is permitted without added complexity of discrete pivoting joints. In addition, adjustment of bar separation, at either or both ends during or subsequent to placement, is simpler, and more readily adapted to minimally or non-invasive techniques. Furthermore, minimally invasive placement may be facilitated by use of an initially placed tether or tethers as a guide and traction mechanism for main segment 10 positioning, as shown for example in FIGS. 46A, 46B, 47A, 47B, 48A, 48A, 49A, and 49B.

FIGS. 46A-53 illustrate several embodiments of devices and components of remodeling devices according to the present invention. FIGS. 46A-50B each have a part “A” and a part “B,” part “A” showing the heart in perspective view through various stages of clasp placement, and part B showing a longitudinal section at the same stage of the placement. This is a non-limiting example in which placement is about the left ventricle 460 and left atrium 461, and positioning of main segment 10 is on the anterolateral and posteromedial aspects of both these chambers. FIGS. 46A-49B directly illustrate successive stages in a preferred method of placement, as well as the structure of the device.

FIG. 46A shows a tether 462, such as a cable, cord, band, chain, guide wire, and the like, that has been passed longitudinally around the heart. Tether 462 can be passed, for example, from the ventricular apex, along the posteromedial surface of the left ventricle, across the posterior atrioventricular junction, through the oblique sinus between the left and the right pulmonary veins (right side of the left veins, left side of the right veins), through an opening in the pericardial reflection separating the oblique and transverse sinuses, through the left part of the transverse sinus (anterior-superior to the “roof” of the left atrium, on either aspect of the atrial appendage, and posterior-inferior to the left and/or main pulmonary arteries), across the anterior atrioventricular junction, longitudinally across the anterolateral surface of the left ventricle, and returning to the apex.

FIG. 46A further shows that one end of this tether is attached to what is to become the atrial end of main segment 10. In another embodiment, the main segment 10 may have a channel (open or closed) from one end to the other which allows main segment 10 to be threaded onto a tether 462 after the placement described above.

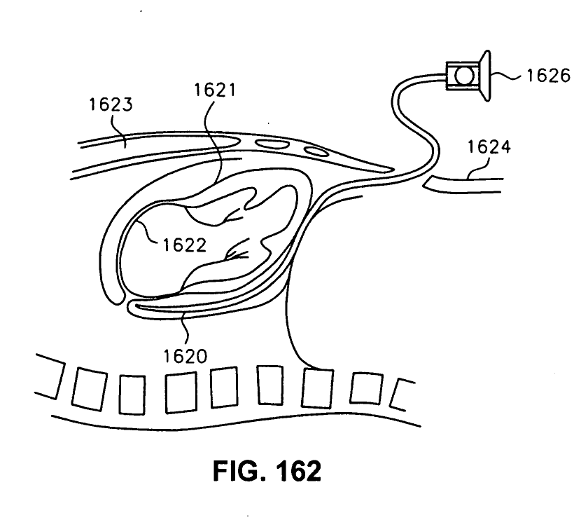

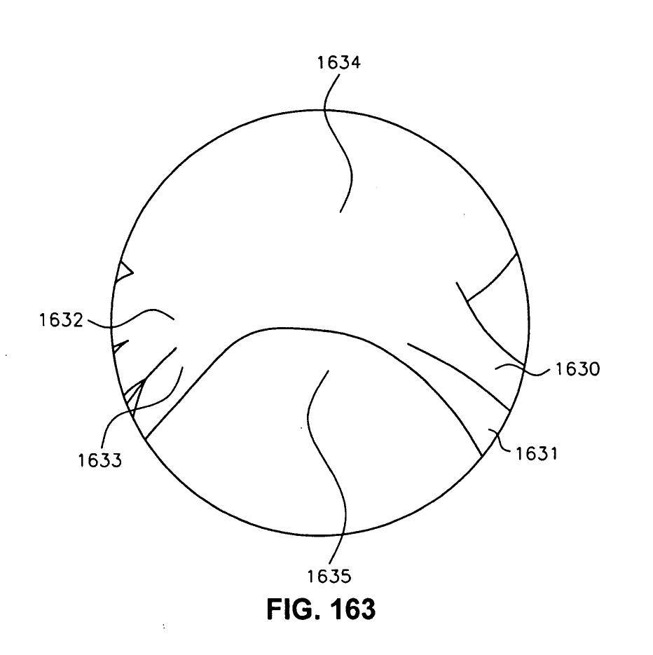

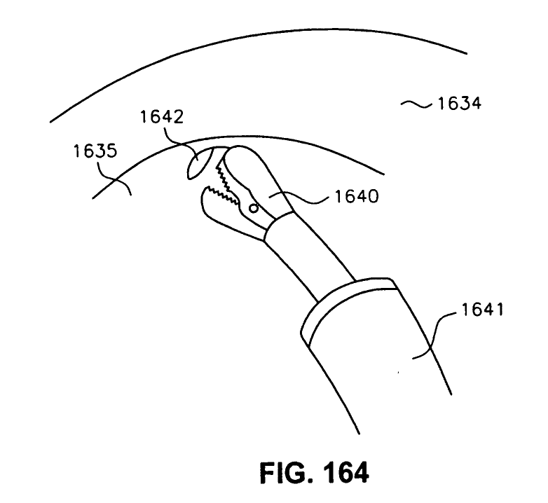

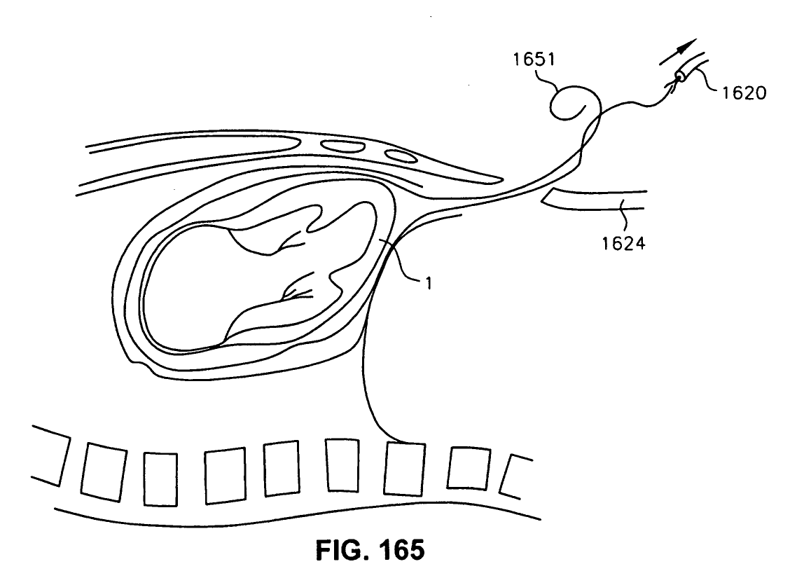

A non-limiting example of a placement method includes placement of an endosurgical access port into the pericardial cavity and introduction of a flexible endoscope through that port as described below (see FIG. 162). The scope could be advanced (with or without supplemental carbon dioxide insufflation and/or positioning the patient with the left posterior chest upward for separation of planes) along the path described above or in the opposite direction, under visual control. Passage through the pericardial reflection may be achieved by either blunt puncture or nibbling via a flexible endoscopic forceps, such as a grasping or biopsy type as described below (see FIGS. 163 and 164). Then, with the port withdrawn, the scope tip may re-exit the pericardial space along side its entry through the port incision. Next, one end of tether 462 (cable or other type) could be grasped by a flexible endoscopic grasping forceps and pulled around the heart as the endoscope is withdrawn as described below (see FIG. 165).

Another potential non-limiting example of a placement method includes the use of multiple ports, including one with a video camera and one or more with grasping, pulling, or other manipulating instruments, with or without ancillary CO₂ insufflation.

It is anticipated that imaging techniques, including ultrasonic (transesophageal, surface, or other). magnetic resonance imaging, and x-ray fluoroscopic methods, can also be used to facilitate accuracy and/or ease of placement of tether 462 or subsequently placed components such as main segment 10.

Localized areas or elements of difference radiopacity or ultrasonic response from surrounding areas or elements may be selectively located on the elements to facilitate placement of elements, relative placement of mating members or longitudinal or radial orientation of elements. The latter may be facilitated by configuration of differential localized areas in shapes which vary with rotational orientation.

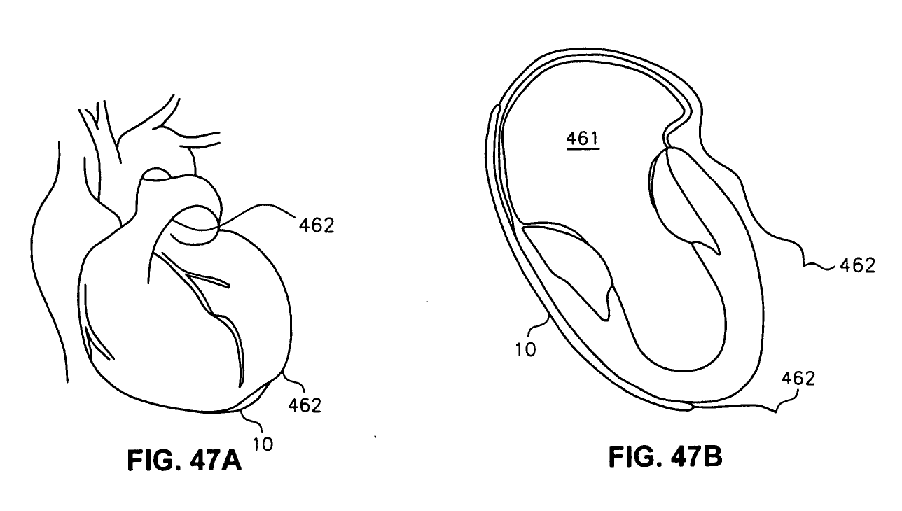

FIG. 47A illustrates that traction on tether 462 may pull the main segment 10 (e.g., posterior main segment 10) into position below and behind the heart chambers. In one embodiment, a second tether 472 (not shown) can be attached to the opposite end of posterior main segment 10 and that end of second tether 472 can be pulled into the pericardial space along with posterior main segment 10 and an anterior main segment 10 could be slid into position along second tether 472.

In the alternative noted above (of the single tether and non-attached but channel-containing posterior main segment 10), posterior main segment 10 can be threaded onto tether 462 and pushed into position along tether 462 while tether 462 is held stationary.

In either case, an incision whose circumference was, or could be stretched to, the circumference of posterior main segment 10 and any auxiliary parts, would suffice. That incision could be subxiphoid or intercostal near the ventricular apex or basal section of heart, as non-limiting examples.

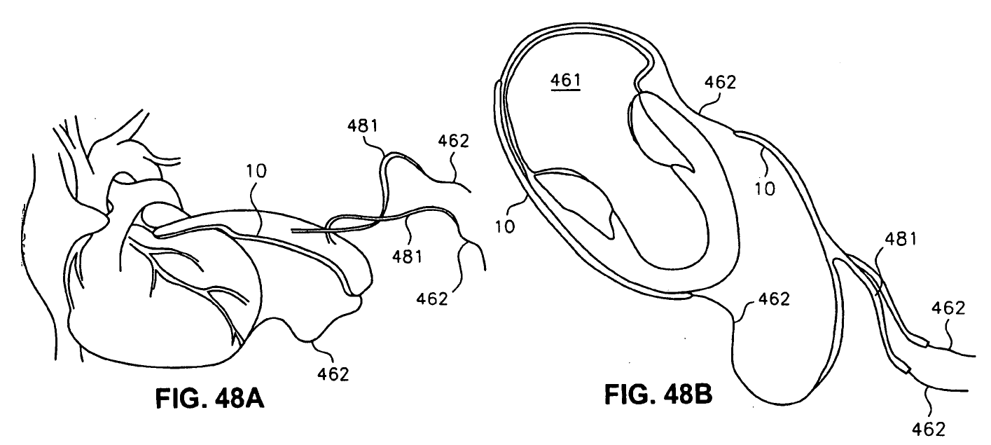

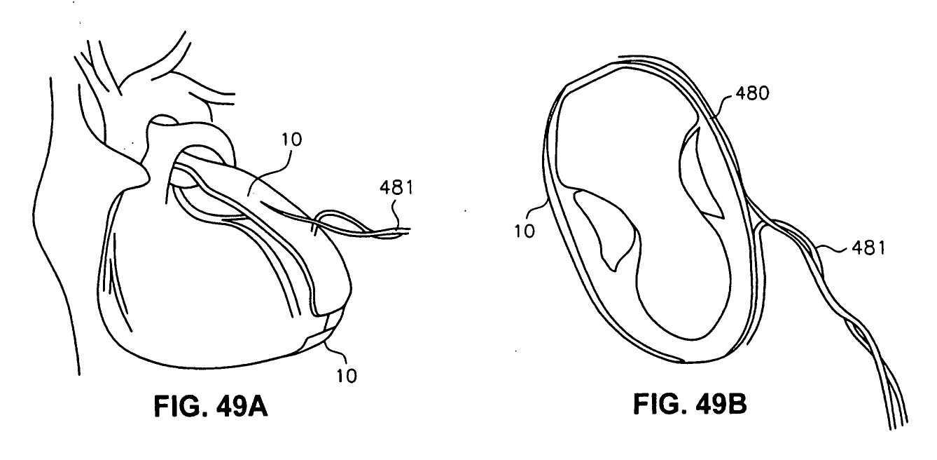

FIGS. 48A-49B show an anterior main segment 10, which has two channels 480 (for example, as shown in FIG. 49B) within main segment 10, one exiting either end, being threaded onto two ends of tether 462, respectively. Each of channels 480 in anterior main segment 10 has an outer end. For a clasp intended to be placed in an open operation, the openings may be in the outer surface of the bar. In a preferred embodiment, where a heart remodeling clasp is intended to be placed in a minimally invasive operation, or a mini-incision operation, the openings of the anterior main segment 10 would continue into a sheath or carrier 481 that is quite limp flexurally but stiff compressively. In either case, the separation distance of the anterior main segment 10 from the posterior main segment 10, at either end, may be adjusted at time of or subsequent to clasp placement, by advancing or withdrawing tether 462 into or out of the carrier sheath at its outer end.

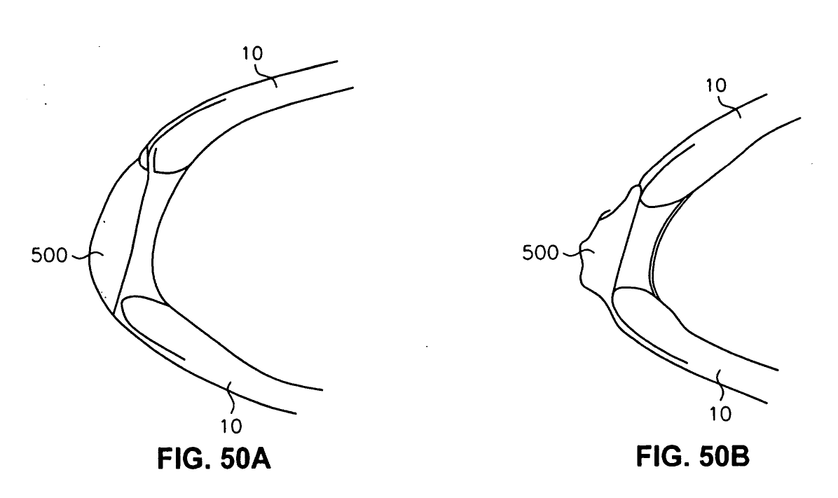

FIGS. 50A and 50B show an spacer or encasement 500 (e.g., formed of elastomeric material) placed at one or both ends between two main segments 10, surrounding tether 462 between the generally rigid main segments 10. During initial or subsequent tether length adjustment, spacer or encasement 500 can be compressed to varying degrees. The purpose of spacer or encasement 500 is to minimize potential tissue trauma by means of increasing the bearing area contacting the heart and other tissues. In addition, the separation of tether 462 from adjacent cardiac or noncardiac tissue or structures achieves a distribution of force and/or affects tissue response in order to reduce or eliminate risk of trauma to such tissue or structures. Spacer or encasement 500 does not substantially compromise either the freedom of length adjusunent of tether 462 or the effect of such adjustment on the net force delivered to the ends of the main segments 10.

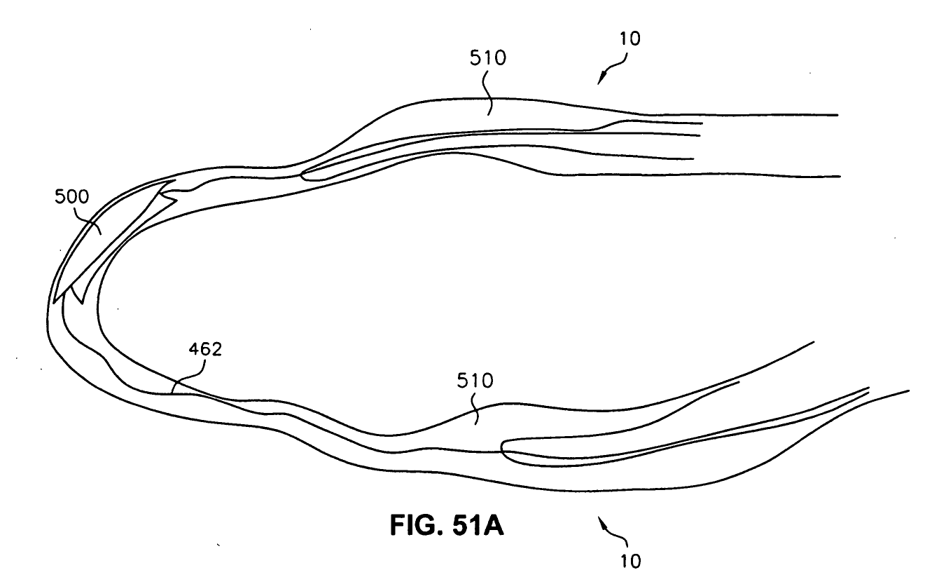

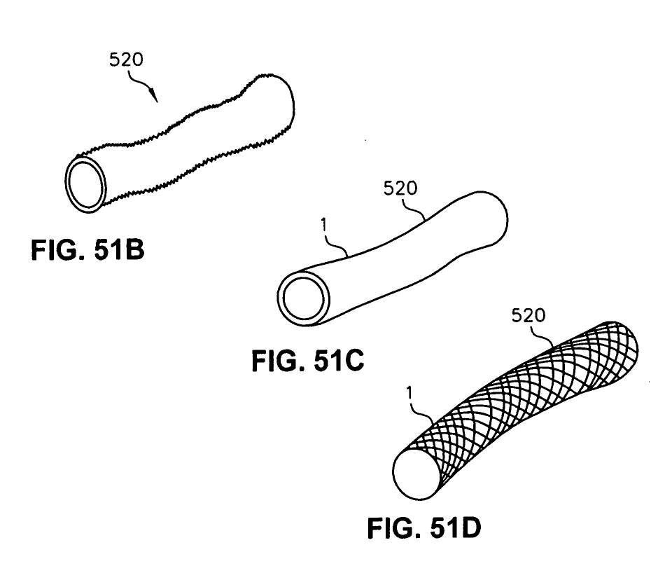

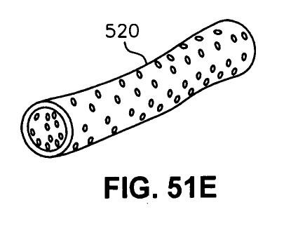

FIG. 51A shows a variation in which a tubular enclosing sheath 510, for example of either a solution-cast elastomer or one of the several materials successfully used for vascular grafts (knitted or woven polyester or expanded PTFE, for example) or other materials, is placed over tether 462, either at the time of tether insertion or subsequent to insertion of a heart remodeling clasp placement. Main segment 10, with or without spacer or encasement 500, are then inserted over tether 462 and within sheath 510. Sheath 510 may be of uniform diameter, but is preferentially of varied caliber to fit the varied component circumferences. In the case of caliber variation, it may be necessary for sheath 510 to be sufficiently elastic to allow passage of larger members.

FIGS. 51B-51E illustrate additional embodiments of spacer or encasement 500. FIG. 51b illustrates a tube 520 which is made from a porous material that is of stable circumferential dimension but freely compliant in length (within a desired predetermined operating range) to applied compressive or tensile force. An example criterion for free length compliance is, for example, that tube 520 alone will require less than 0.1N of either tensile or compressive force to either lengthen or shorten, respectively, the entire range of its operation.

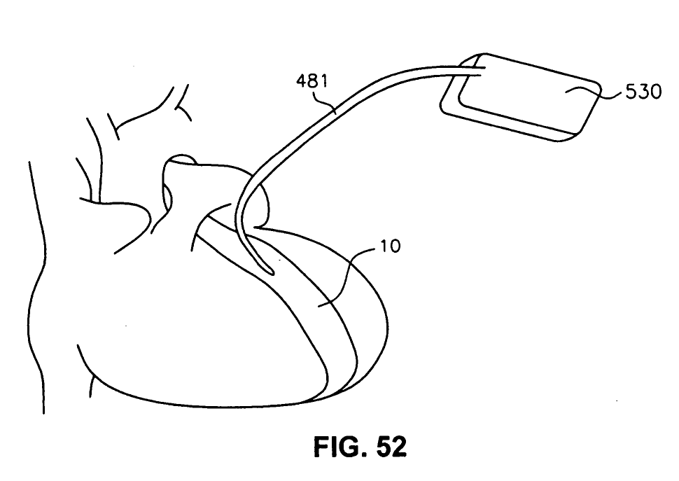

Examples of spacer or encasement 500 include tubes shown in FIGS. 51B-51E. FIG. 51B shows, as noted above, a tube 520 made of porous, surface crimped corrugated fabric such as commercially knitted, woven, or braided vascular prostheses or custom-fabricated approximations of such tubes. A typical material of construction is polyester. Expanded polytetrafluoroethylene (PTFE) tubes without outer membrane jackets or other reinforcement means are also useable (as shown in FIG. 51c), as are woven or loosely (e.g. <20 yarn-count/inch) diagonal-braided yarn tubes (as shown in FIG. 51d). FIG. 51e illustrates a tube 520 as shown in FIG. 51c and having holes or perforations (such as round, rectangular, diamond shaped, etc.) along its wall to allow for tissue ingrowth after placement. FIG. 52 shows the addition of an adjustable control mechanism 26 including adjustability canister 530 (for example, for adjusting a distance between main segments 10 and/or the size and shape of stabilizer/reconfiguration segment 12), which may be placed at some distance from the heart such as, for example, the subcutaneous tissue of the abdomen or prepectoral region. FIG. 53 shows another perspective of such a clasp with adjustability canister 530.

Adjustability canister 530 can be used to adjust by non-invasive, minimally invasive, and/or invasive procedures, a distance between main segments 10 and/or the size and shape of stabilizer/reconfiguration segment 12. Canister 530 can be accessed, for example, under local anesthesia by an open incision that allows tightening or loosening of a screw mechanism by an instrument (e.g., allen wrench or screwdriver) to advance or retract the length of tether 462. Canister 530 can also be accessed under local anesthesia and a skin/tissue-penetrating instrument such as a flat or triangular tipped (Keith) surgical needle used to engage a screw mechanism through a self-sealing elastomeric plug. Canister 530 could also contain a ratchet mechanism with a permanent magnet affixed, so that a varying magnetic field at skin surface, generated either by a moving a permanent magnet or a solenoid, may advance or retract the length of tether 462. In addition, canister 530 can have a compressible diaphragm on the surface nearest the skin, which may be cyclically compressed, engaging a ratchet mechanism to advance or retract the length of tether 462. Furthermore, canister 530 can have an electrochemical cell (batteries), geared electric motor, and appropriate assembly, that when actuated may advance or retract length of tether 462. In one embodiment, adjustable control mechanism is programmable from outside by radio or magnetic signals such as used in programmable pacemakers or radio-controlled toys in ways familiar to those experienced in these fields of technology. The adjustable control mechanism may include position sensors and electronics for telemetric detection of position by the programming device. In that event, it may or may not have a feed-back servo mechanism whereby the external programmer may have the desired position or desired movement or desired force entered as a digital or analog signal.

Alternate Heart Remodeling Clasp

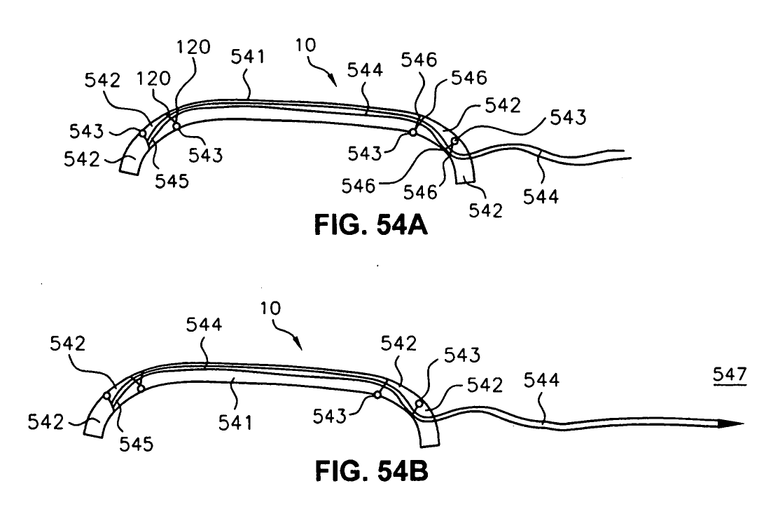

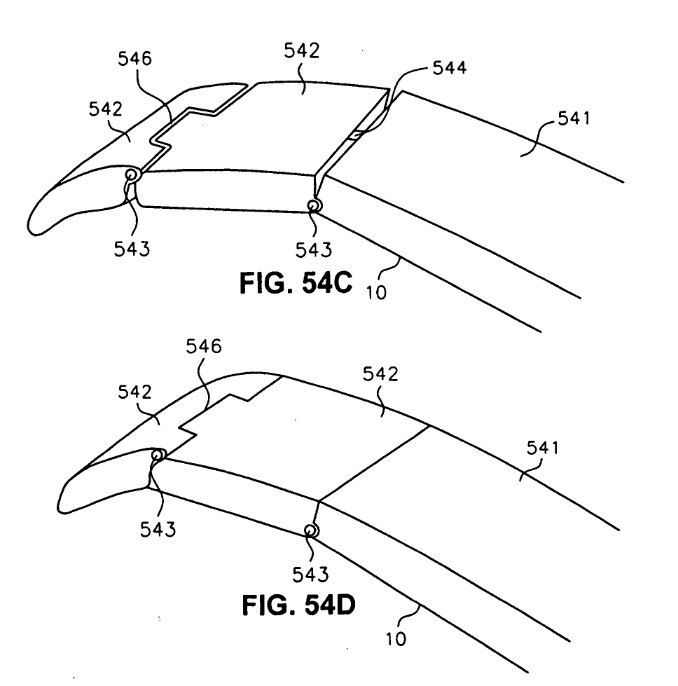

FIG. 54A shows one embodiment of an improved type of main segment 10 of a heart-remodeling clasp according to the present invention. It is similar to other main segments 10 in that it employs bimeridianal restraining segments to reduce the wall-tension/chamber-pressure ratio. Bimeridianal restraining segments include middle segment 541, and one or more shoulder sections 542 connected together and to middle segment 541 by hinges 543. In one embodiment, a traction cable 544 is anchored to one of end segments 542 at point 545 and passes through shoulder segments 542 and segments 541, 542 via openings 546. In one embodiment, openings 546 are located opposite hinges 543 as shown in FIG. 54B.

As traction cable 544 is tensioned and pulled through openings 546 in the direction of arrow 547, shoulder segments 542 and bimeridianal restraining segments 540 are configured into the position shown in FIG. 54B where hinges 543 are closed. As the tension on traction cable 544 is released, the bimeridianal restraining segments 540 can return to the position shown in FIG. 54A. By tensioning or releasing the tension on traction cable 544, bimeridianal restraining segments 541, 542 on the natural heart surface can be tensioned or released to the desired position to accommodate and/or assist systolic and diastolic function of the heart.

FIGS. 54E and 54F show an embodiment of main segment 10 such as that shown in FIGS. 54A and 54B except the relative width of each segment is larger.

Adjustable Stabilizing and/or Reconfiguration Segments

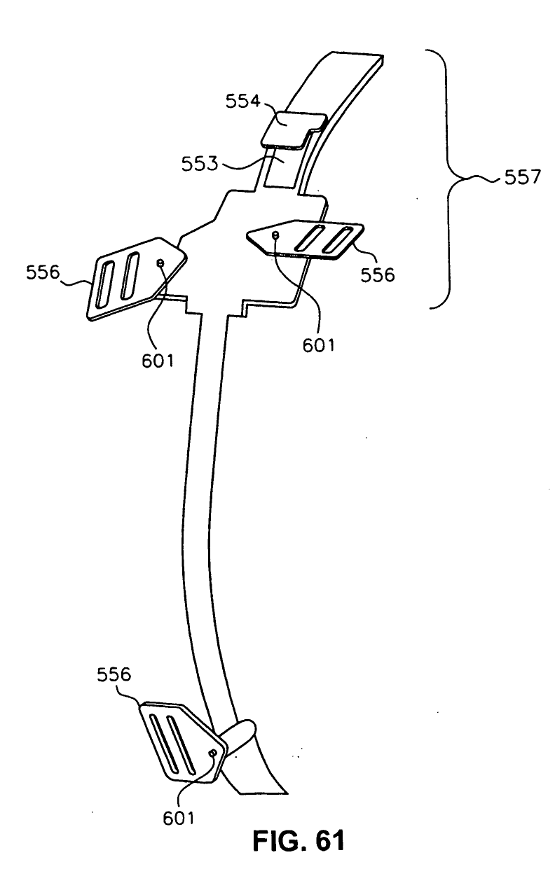

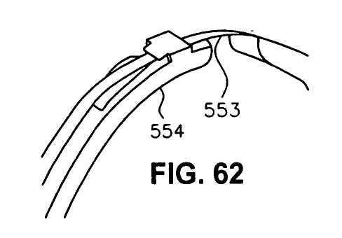

In one embodiment, as shown in FIG. 55, a heart remodeling clasp according to the present invention includes main segment 10 having compression segment 550, shoulder segments 551, 557, and adjustable closure 552. Compression segment 550, for example, includes in one embodiment the features of segment plates 170 shown in the FIGS. 17-31. Adjustable closure 552 can be any adjustable closure that will join main segments 10 and compression segments 550 at the top and bottom of the clasp. In one embodiment, adjustable closure 552 includes adjustable cable or strap 553, and releasable lock 554, as shown more specifically in FIGS. 61 and 62.

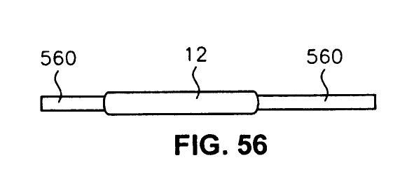

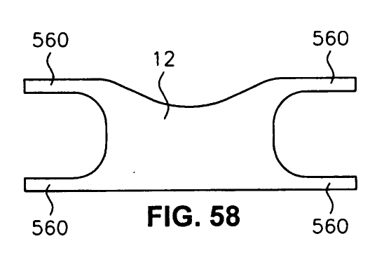

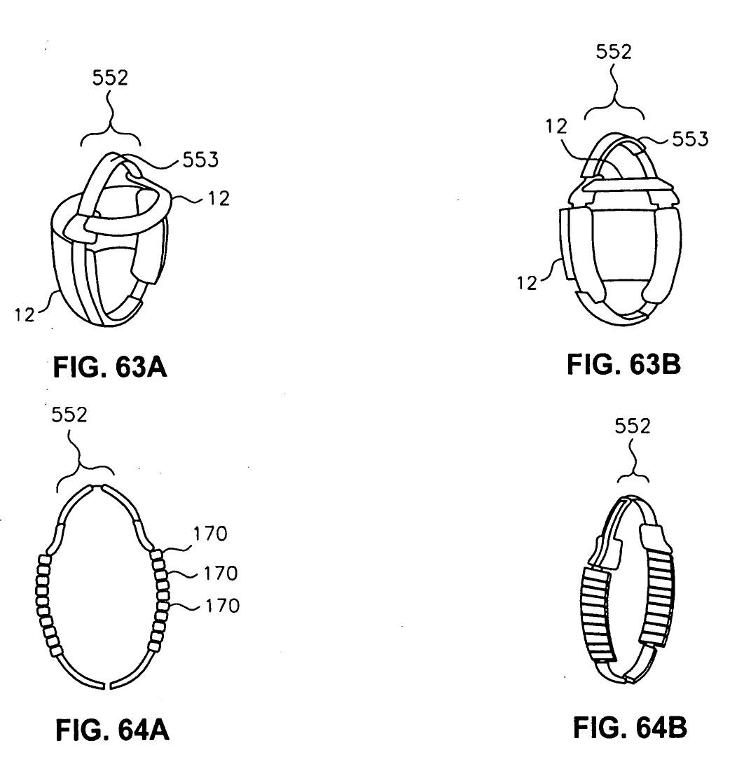

The heart remodeling clasp according the present invention can also be used with adjustable stabilizer/reconfiguration segments 12 as shown in FIGS. 56 and 58. Adjustable stabilizer/reconfiguration segment 12 are used to (a) stabilize the main segment 10 in position on die natural heart as shown, for example, in FIGS. 63a and 63b and/or (b) to reconfigure one or more portions of the natural heart as shown in, for example, FIGS. 5, 7, 8, 10A, 20B, 11A, 11B, 12A, 12B, 13A, 13B, 14A, and 14B.

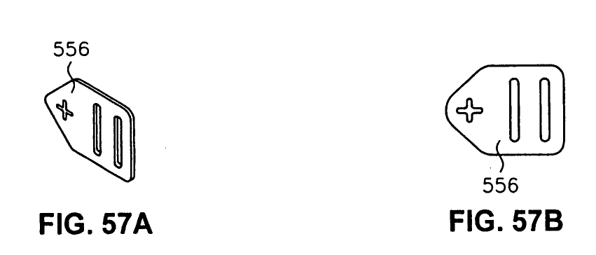

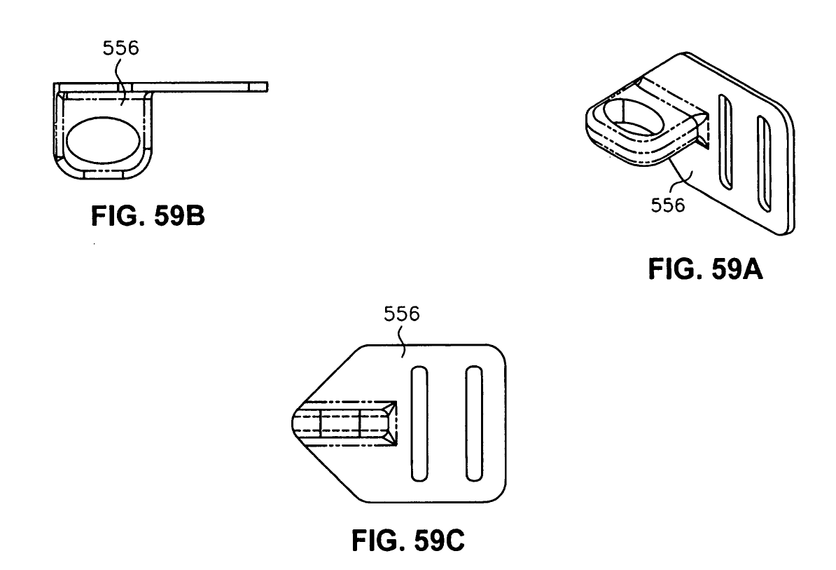

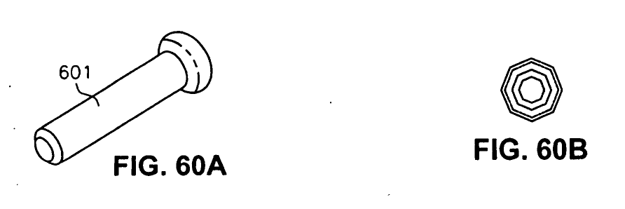

Adjustable stabilizer/reconfiguration segments 12 are configured to fit the particular shape of the portion of the natural heart on which they are to be located. For example, adjustable stabilizer/reconfiguration segments 12 can be configured as shown in FIGS. 56, 58, 63A, 63B, or as shown, for example, in FIGS. 5, 7, 8, 10A, 10B, 11A, 11B, 12A, 12B, 13A, 13B, 14A and 14B. Adjustable stabilizer/reconfiguration segment 12 is flexible, semi-rigid or rigid depending on intended placement and use thereof. In one embodiment, adjustable stabilizer/reconfiguration segment 12 is attached to the clasp by slipping ends 560 (as shown in FIGS. 56 or 58) thereof through attachment clips 556 or any other means for adjustably attaching stabilizer/reconfiguring segment(s) 12 to the clasp. Attachment clips 556 are configured as shown in FIGS. 55, 57a, 57b, 59a, 59b, and 59c and are attached to the clasp via attachment pins 601 (shown in FIG. 60) at a location on the clasp to achieve the desired stabilization and/or reconfiguration. For example, attachment clips 556 can be attached adjacent the shoulder segment 557 or at any point along the compression segment 550, as shown in FIG. 55.

It should also be noted that the spacers or encasements 520 discussed above with respect to FIGS. 50A and 51A-51E, could also be used to cover adjustable cable or strap 553, or any other part of the main segment 10 or adjustable stabilizer/reconfiguration segment 12 where the direct contact of the heart is undesirable.

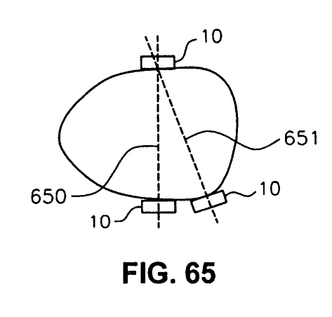

In one embodiment shown in FIG. 55, shoulder 557 is configured to fit adjacent the atrioventricular groove and compression segment 550 is configured to fit adjacent (e.g., on) the left ventricle. If main segment 10 starts to slip off the natural heart 1, tension in adjustable stabilizer/reconfiguration segment 12 created by such slippage increases to prevent main segment 10 from slipping off the natural heart or a portion thereof, as shown diagrammatically in FIGS. 65-67.

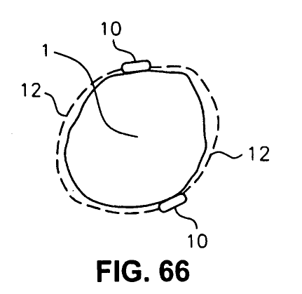

FIG. 65 shows two lines of orientation, line 650 which illustrates the situation where main segments 10 are positioned 180° from each other, and line 651 which illustrates an off-center positioning between main segments 10. The degree of offset can vary, but is preferably is in the range of between 145° and 180°. In FIG. 66, main segments 10 are held in place by one or more pieces of material making up stabilizer/reconfiguration segment 12 on the lateral side of the heart and one or more additional pieces of material making up stabilizer/reconfiguration segment 12 on the right ventricular side of the heart.

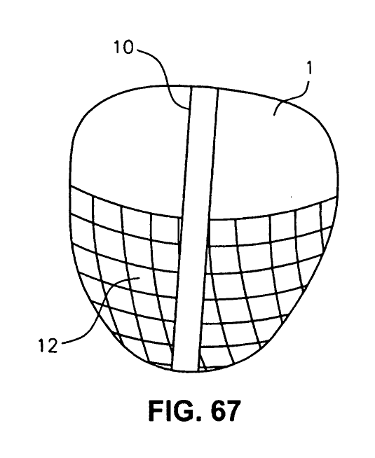

FIG. 67 shows the same embodiment as illustrated in FIG. 66, but from a side perspective using a stabilizer/reconfiguration segment 12 that is relatively wide compared to the size of the heart being treated. The orientation of main segments 10 can be placed on a heart without regard to the internal structure of the heart as required for devices internal to the heart. Accordingly, main segments 10 can be placed on the heart and achieve increased heart function (e.g., increased ejection fraction and decreased valvular regurgitation), as are not experienced with many internal devices.

All elements are configured to fit the particular portion of the heart on which they are to be placed. For example, as shown in FIGS. 63a, 63b, 64a, and 64b, closure segments 552 can be configured to bridge the basal portions and apical portions of the natural heart.

Alternative Adjustable Stabilizing/Reconfiguration Segments Clasp with Pacing Leads

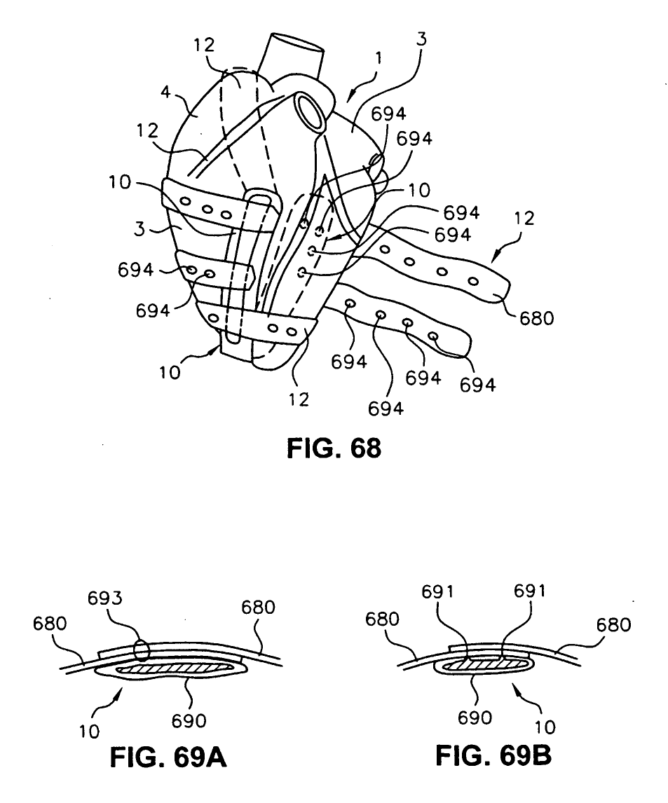

The present invention is also directed to an adjustable stabilizing/reconfiguration segment 12 for use with transceivers or pacing leads 694 capable of receiving and transmitting electrical signals, for example from a pacemaker. Referring to the figures, an exemplary natural heart 1 is shown in FIGS. 68, 70 and 71.

A natural heart 1 has a lower portion comprising two chambers, namely a left ventricle 2 and a right ventricle 3, which function primarily to supply the main force that propels blood to and from the lungs, and the peripheral circulatory system, which propels blood through the remainder of the body. Natural heart 1 also includes an upper portion having two chambers, a left atrium 3 and a right atrium 4, which serve as an entryway to the left and right ventricles 2 and 3, respectively. As shown in FIG. 68, adjustable stabilizing/reconfiguring segment 12 includes one or more straps 680 (e.g., which may be suturable) which encircle the heart and are secured to any one or more of the main segments 10 described in this application, including a U shaped member segment as more fully described in U.S. patent application Ser. No. 08/035,710, incorporated herein by reference, with sutures.

FIGS. 69A and 69B show alternate constructions of the main segment 10 and straps 680. In FIG. 69A, a cross-section is shown in which main segment 10 is encased in a suturable material encasement 690 such as a porous or non-porous material such as polyester mesh, woven polyester, silicone rubber, polyester fabric or reinforced silicone. Encasement 690 about main segment 10 provides a means for attaching straps 680 to main segment 10, which itself may be formed of material that would accept a suture. In FIG. 69B, main segment 10 is formed such that its exterior surface includes encasement 690, shown held in between two projections 691 in main segment 10. In this embodiment of the present invention, sutures 693 may be passed through straps 680 into encasement 690 held to main segment 10. Sutures 693 (not shown) in both FIGS. 69a and 69b.

As shown in FIG. 68, several adjustable stabilizing/reconfiguration segments 12 may be used to help maintain main segments 10 in position on the natural heart. FIG. 68 shows three adjustable stabilizing/reconfiguration segments 12 in position with two additional adjustable stabilizing/reconfiguration segment 12 crossing over the top of the natural heart. Thus, in this embodiment, five (5) stabilizing/reconfiguration segments 12 are used.

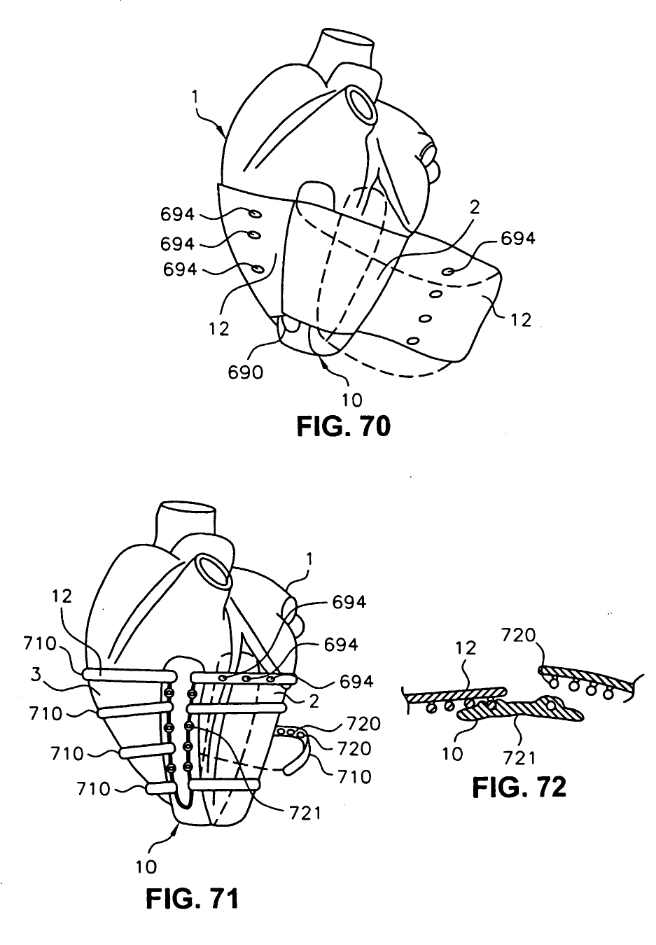

As shown in FIG. 70, the anchoring of adjustable stabilizing/reconfiguration segments 12 may take the form of a soft harness such as porous (e.g., a suturable mesh) or non-porous material. In this embodiment, adjustable stabilizing/reconfiguration segments 12 are wrapped about the natural heart and sutured to a suturable material encasement 690 of the main segment 10 as shown in FIGS. 69A and 69B. Adjustable stabilizing/reconfiguration segment 12, for example, may be formed of any biocompatible material and may be relatively narrow or may cover a relatively wide swath across the natural heart as desired by the surgeon.

As shown in FIGS. 71 and 72, adjustable stabilizing/reconfiguration segments 12 alternatively include one or more rigid, semi-rigid or flexible bands 710 that are designed to encircle the heart and include clamping mechanism 720, or the like, at each end of adjustable stabilizing/reconfiguration segments 12 which cooperate with an engagement mechanism 721 attached to or integral with main segment 10. As shown in this embodiment, clamping mechanisms 720 are ball snaps 722 which engage receptacles 723 in the engagement mechanism 721. In this form of the present invention, entire band 710 may be formed of a rigid, semi-rigid or flexible material. Alternatively, the ends thereof might be formed of such a material and the remainder of the band 710 may be configured like straps 680 as shown in FIGS. 68 and 69a, with the clamping mechanisms 720 being as shown in FIG. 72. In addition, any other type of adjustable attachment mechanism or non-adjustable mechanism, such as clamps, may be used to secure adjustable stabilizing/reconfiguration segments 12 to main segment 10.

In certain embodiments of adjustable stabilizing/reconfiguration segment 12 according to the present invention, several distinct regions are formed which may be utilized to hold and carry transceivers or pacing leads 694 which extend from or through the adjustable stabilizing/reconfiguration segments 12. Transceivers or pacing leads 694 also can be placed on the main segment 10 as shown in FIG. 68 in phantom. There may be one or more pacing leads and/or transceiver elements (e.g., elements capable of sending and receiving, both from the heart and electrical devices, electrical signals) as desired such that pacing or other manipulation or diagnosis of the heart may be readily accomplished.

In some cases, the stabilizer/reconfiguration segment may be sized to be slightly shorter than the exterior heart wall which it traverses so that it exerts a continual inward pressure on the wall and thus serves to reconfigure the heart in that location. In other embodiments, the stabilizer/reconfiguration segment is sized to exert little or no inward force on the heart wall and thus serves only as a stabilizer element.

Catheter Based System to Reduce Myocardial Wall Tension

The present invention is also directed to a method for placing restructuring or other devices into one or more chambers of the heart. In one embodiment, the method according to the present invention includes a catheter based system that may be used to place a system such as that shown in U.S. patent application Ser. No. 08/035,710 or U.S. Pat. No. 5,961,440, both of which are hereby incorporated by reference.

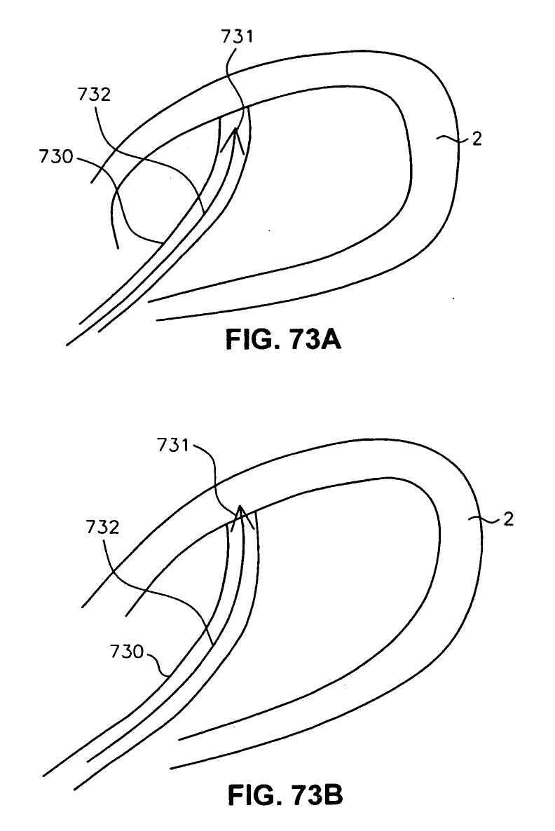

In the present method, as shown in FIGS. 73A-75B, via an artery leading to the ventricle, a catheter 730 is positioned within the left ventricle 2 in a non-invasive or minimally invasive procedure. A reversibly collapsible anchor 731 in the form of a clamshell or umbrella in its collapsed form is pushed outwardly through the left wall of left ventricular 2. This insertion of a reversibly collapsible anchor 731 through the wall may be aided with intravascular ultrasound. Once through the wall, anchor 731 opens to provide a nail or rivet-like planar surface that is then pulled back against the external surface of the wall. The same deployment of a second anchor 731 occurs on another portion of the wall of the left ventricle 2, for example on the wall of left ventricle 2 opposing the location of first anchor 731. Wires, cables or cords 732 attached to the anchors 731 are then connected and tightened, thereby decreasing this left ventricular dimension, and exerting a continual inward pull on the chamber walls, indenting the walls and reconfiguring the chamber. In one embodiment, a single wire, cable or cord 732 is used.

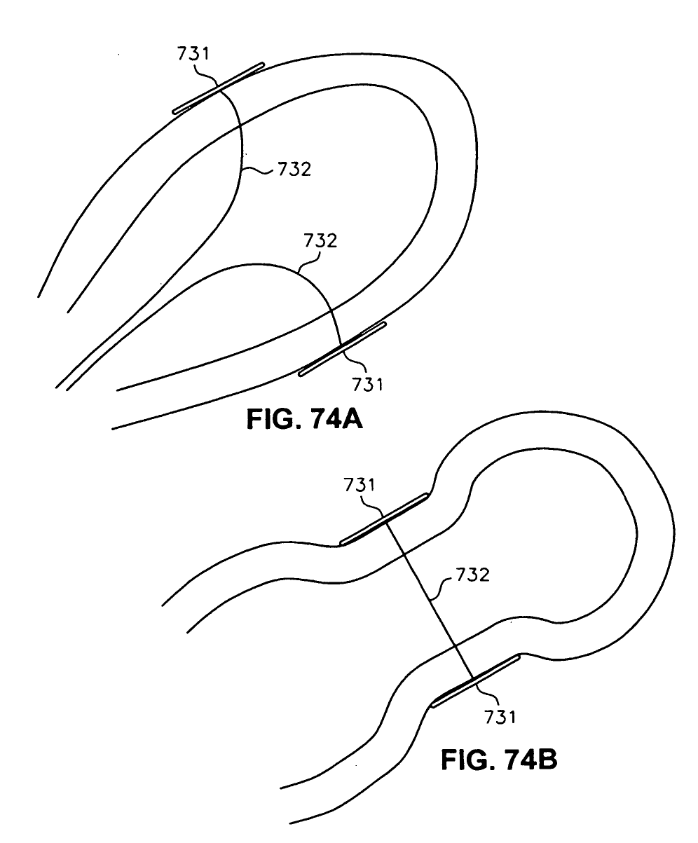

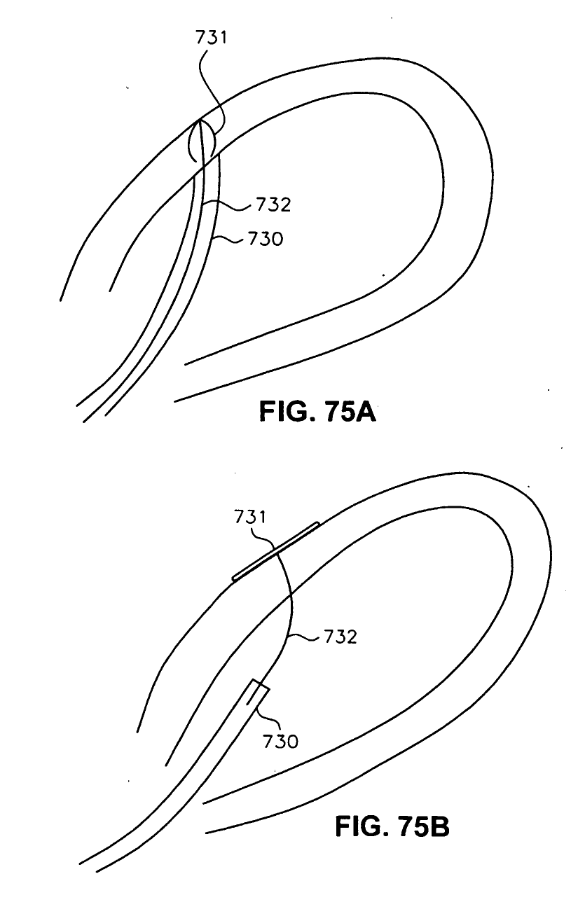

FIG. 74A shows anchor 731 open against the exterior wall of the left ventricle 2 after the two cords 732 have been placed. FIG. 74B shows the final cord 732 after joining and tightening of the two cords 732 originally placed. FIGS. 75A and 75B show clamshell anchoring mechanisms which work in the same manner as the umbrella embodiment described above. The umbrella-like anchor may also include a head which when elongated is an elongated planar configuration rather than round so that pressure applied against the exterior surface of the heart creates an elongated indentation in the chamber. umbrella-like anchor may also include a head which when elongated is an elongated planar configuration rather than round so that pressure applied against the exterior surface of the heart creates an elongated indentation in the chamber.

By using the method of inserting transventricular reconfiguration members described above according to the present invention, the surgeon can avoid opening the patient's chest wall.

Delayed-Penetration Pegs for Epicardial Fixation

In certain embodiments, the invention also provides local stabilization and/or fixation of elements of heart remodeling clasp-type reconfiguration devices according to the present invention. Such elements may include elements that assist in stabilizing a surface of a natural heart. As shown in FIGS. 76a, 76b and 76c, cross-sections of clasps according to the present invention (for example those shown in FIGS. 1a, 3, 7, 10A, 10B, 53, 55,) can be stabilized and/or fixed to the surface of the natural heart by one or more stabilization protrusions 174 in the form of pegs or studs designed for delayed penetration into the natural heart surface 1. Stabilization protrusions 174 may be attached to or integral with main segment 10 and/or adjustable stabilization/reconfiguration segment 12.

Stabilization protrusion 174 is particularly adapted to devices which, by their nature, are kept pressed against the natural heart surface 1 and for which the major risk is tangential displacement.

Stabilization protrusions 174, for example, have three main embodiments: (1) permanent protrusions or pegs; (2) fully or partially absorbable protrusions or pegs; and (3) extendable protrusions or pegs; and combination of the same. Extendable protrusions or pegs 174 can be either permanent or partially absorbable.

The principle of the stabilization protrusion 174 according to the present invention is as follows. The length of stabilization protrusion 174 is somewhat longer than the diameter of stabilization protrusion 174. Stabilization protrusion 174 can be of any cross sectional profile. A preferred profile is generally circular, with a relatively blunt hemispheric tip.

In one embodiment, more than one stabilization protrusion 174 is formed integral with main segment 10 in a single line along the length of stabilization protrusion 174. Each stabilization protrusions 174 are separated from one another by a space, for example, at least twice the length of an individual stabilization protrusion 174. Due to differing heart wall thicknesses of an individual, optimal penetration of stabilization protrusion 174 into natural heart surface 1 is determined experimentally. The maximum stabilization effect is thought to occur at the maximum penetration of stabilization protrusion 174 that will not damage the epicardium during brief (e.g., approximately <15 minutes) trial placements. This strategy is intended to allow movement one or more times during the placement operation, based on gross, echocardiographic, or other assessment.

Stabilization protrusions 174 are thought to work because initially the relatively tough epicardial layer of natural heart surface 1 is deformed at the site of pressure by stabilization protrusions 174 in a tent-like fashion downward into the natural heart surface, as shown in FIG. 76B. The muscle fibers and blood vessels 761 are free to move for short distances and will be displaced to one or the other side without damage. The ‘tented’ epicardium, so viscoelastically deformed, acts to counter potentially displacing tangential forces and thus to stabilize in position. Referring to stabilization protrusion 174, pressure on the very small surface area at the tip of stabilization protrusion 174 is quite high, approximately 1 to 5 megaPascals (7,500 to 37,500 mmHg). This pressure causes very localized tissue death or necrosis followed by loss of mechanical integrity. The epicardium will then separate, and the margins of the hole created in the epicardium surround the sides of the stabilization protrusion 174 toward the bar as shown in FIG. 76c. At this time, the muscle fibers and blood vessels 761 continue to be displaced to the sides of stabilization protrusion 174. Position stabilization for stabilization protrusion 174, and thus of the main segment 10 or stabilization/reconfiguration segment 12, is maintained.

There is a tendency for devices such as heart remodeling clasps including main segments 10 and/or stabilizer/reconfiguration segment 12, according to the present invention which are applied to the surface of the heart to become displaced tangentially due to the motion of the heart. This has particularly been observed, for example, in the acute experimental trials of clasps according to the present invention, in the absence of such local stabilization means.

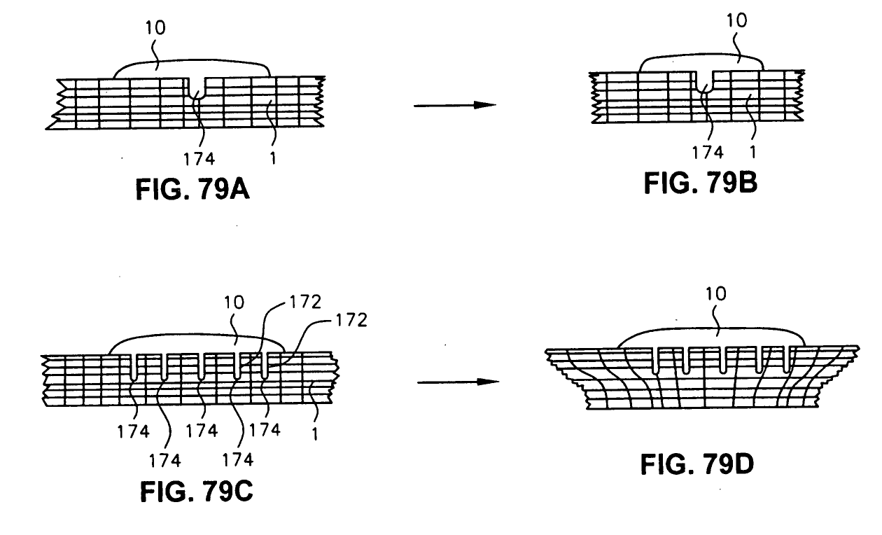

The likelihood is that a broad-based area of fixation of an epicardial-contacting device would ‘splint’ or immobilize the layers of myocardium immediately subjacent to the device, such that part of the muscle mass could not effectively contribute to heart function. This could occur with stabilization protrusion 174 if placed along the width of main segment 10 as shown in FIGS. 79C and 79D. Accordingly, in one embodiment stabilization protrusions 174 are confined to a narrow longitudinal centerline of a device such as main segment 10 of a heart remodeling clasp according the present invention, as shown in FIG. 79a. In FIG. 79a, only the first of multiple stabilization protrusions 174 are shown on main segment 10 in a top view in cross-section of main segment 10. In such devices, stabilization protrusions 174 may be an improvement over or used in addition to local fixation means such as adhesives and those methods and devices that promote scar tissue.

Stabilization protrusions 174 are different from sutures in that the protrusions do not require complex manual or instrumental manipulation to place. It is different from tacks or spikes in that blunt configuration of stabilization protrusions 174 delays penetration. It is different from adhesives in that effective fixation is only in the tangential direction and in that local transverse shortening of the heart is not restrained. It is different from methods that promote scar tissue fixation in that stability is immediate.

Relative to sutures, the devices with stabilization segments offers fixation with no complex manual or instrumental manipulation at the site of fixation, which is of great potential value in minimally invasive placement of the devices to be stabilized. Relative to sharp spikes or tacks, risk of coronary damage is expected to be greatly diminished. Relative to adhesives, the tangential-only fixation allows removal and repositioning any number of times without harm during placement, until position is acceptable. Relative to reliance on scar tissue formation, fixation is immediate.

Stabilization protrusions 174 according to the present have several embodiments, including permanent pegs, fully or partially absorbable pegs, and extendable pegs or combinations of the same. The permanent relatively blunt stabilization protrusions 174 (such as pegs) are rigid, nonabsorbable posts of the type shown in FIGS. 76A-76C, which extend, generally perpendicularly, toward the natural heart surface 1 from main segment 10.

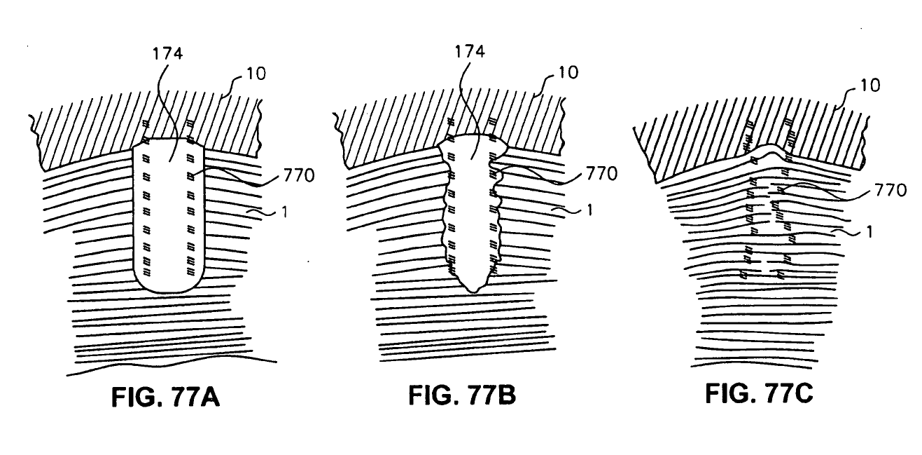

In another embodiment, as shown in FIGS. 77A, 77B and 77C, stabilization protrusions 174 are fully or partially absorbable pegs having a rigid component made of a fully or partially absorbable biomaterial. In this embodiment, stabilization protrusions 174 may also include a porous (for example a flexible or rigid) component 770 (shown in cross-section in FIGS. 77A, 77B and 77C) such as a flat or tube-like mesh, wire or net that is not absorbable and which extends into or is attached to main segment 10. The Porous component 770 is embedded in or may surround the rigid or semi-rigid component of stabilization protrusions 174. In this embodiment, the penetration mechanism is as for the stabilization protrusions 174 described above. Stabilization protrusions 174, exposed over time to tissue fluid and the agitation of cardiac motion at all surfaces, begin to dissolve and/or is partially absorbed (FIG. 77B) or fully absorbed (FIG. 77C) by the heart tissue, depending on the material of which stabilization protrusions 174 are composed. If stabilization protrusion 174 includes a flexible porous component exposed before, simultaneous with, and after full or partial absorption of the rigid component, the healing process of the myocardium which has been damaged by fiber separation, may cause collagen fibers to penetrate interstices in the porous component 770.

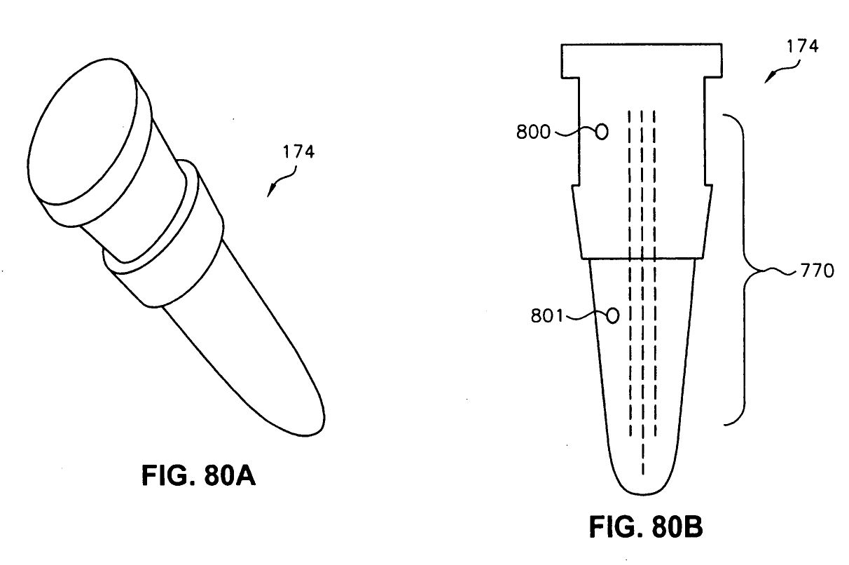

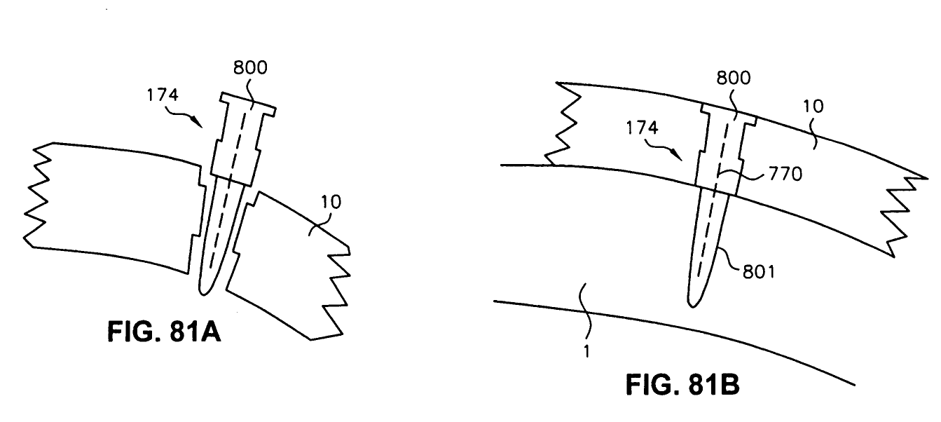

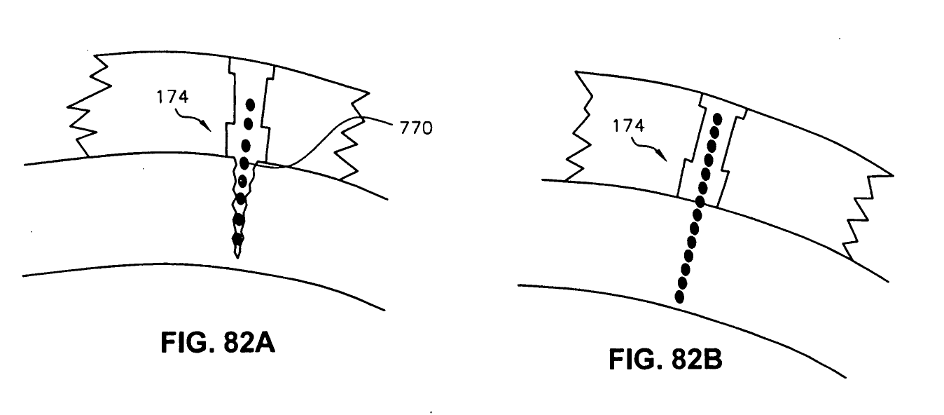

In another version of this embodiment, as shown in FIGS. 80a and 80b, stabilization protrusion 174 includes a rigid or semi-rigid non-absorbable head 800 (e.g., formed of a biocompatible polymer), a rigid or semi-rigid partially or fully absorbable tip 801, and a non-absorbable porous component 770 (e.g., a flexible or rigid mesh, wire or net). As shown in FIGS. 81A and 81B, head 800 is attached to main segment 10 by any mechanical or chemical means. Then, stabilization protrusion 174, by delayed penetration as discussed above with respect to FIGS.76A-76C, penetrates natural heart surface 1 by delayed penetration (the end result of which is shown in FIG. 81B), after which partially or fully absorbable tip 801 is absorbed as shown in FIG. 82A. The healing process of the myocardium which has been damaged by fiber separation causes collagen fibers to penetrate interstices in the porous component 770 as shown in FIG. 82B.

The composition of the stabilization protrusion 174 is selected and/or treated such that it will provide tangential stability of stabilization protrusion 174, and thus of main segment 10, on natural heart 1 until it is fully absorbed i.e., the stabilizing effectiveness of the rigid component continues until it is fully absorbed. The materials for fully or partially absorbable protrusion 174, or portions thereof, will ordinarily be selected to be partially or fully absorbable over a predetermined period of time.

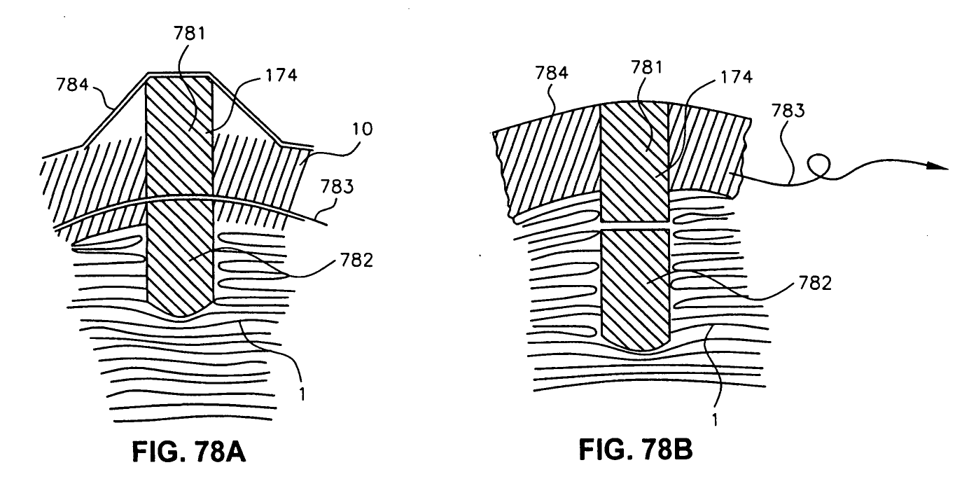

Another embodiment of stabilization protrusion 174, as shown in FIGS. 78a and 78b, according to the present invention is a spring-loaded, length-extending protrusion or peg. According to this embodiment, stabilization protrusions 174 have first and second sections 781 and 782, separated by a releasable holding mechanism 783 such as a wire or similar element, and a spring, elastic or tensioned band or wire 784, or similar element.

Stabilization protrusions 174 are initially engaged with natural heart surface 1 as discussed above up to the length of second section 782. After this initial penetration depth has been achieved, the penetration depth may be increased immediately or after a period of time by removing releasable holding mechanism 783 and allowing band or wire 784 to push stabilization protrusions 174 into natural heart surface 1 to an optimal depth.

This embodiment provides an initial limited penetration in the natural heart surface by stabilization protrusions 174 controlled by releasable holding mechanism 783, which opposes the extending force of band or wire 784. In one embodiment, band or wire 784 is formed of a silicone rubber strip. After main segment 10 is positioned on natural heart surface 1, releasable holding mechanism 783 is released, and the elastic or tension force of band or wire 784 causes stabilization means to penetrate natural heart surface to an optimal predetermined depth. Resistance of muscle fibers to displacement may or may not cause a detectable delay in full penetration.

The material of the spring-loaded or tensioned, length-extending stabilization protrusions 174 may be totally non-absorbable as in the permanent stabilization protrusions 174, and may be porous or non-porous.

The materials forming the stabilization protrusions 174 may be porous or non-porous. A porous material may be used to promote tissue in-growth into stabilization protrusions 174. As discussed above, the materials may also be non-absorbable, or partially or fully absorbable.

Flexible Sheath Containing Rigid Segments and/or Rigid Adjustable Segments

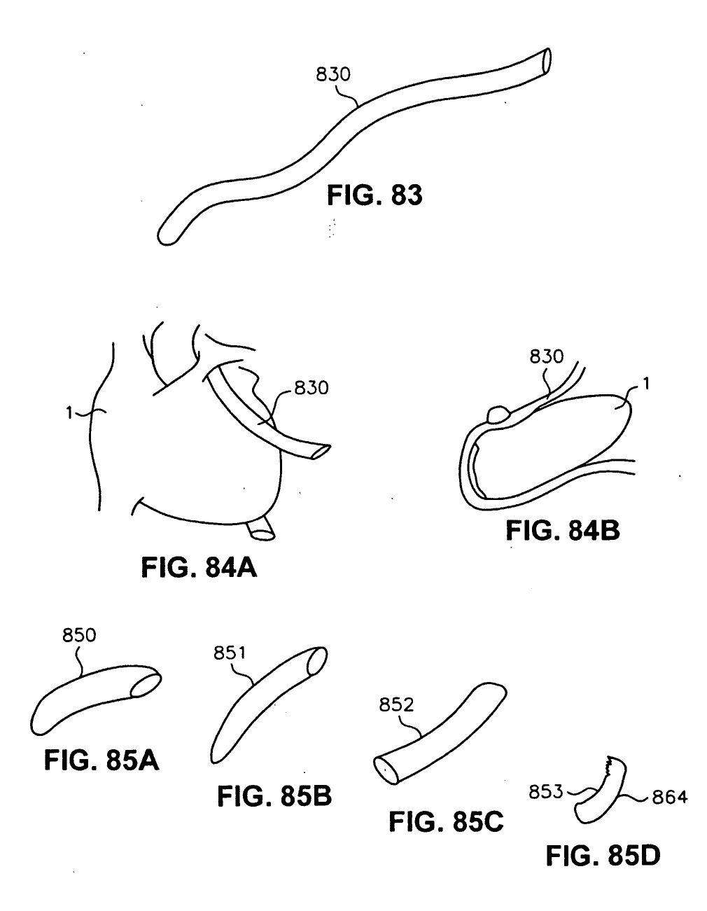

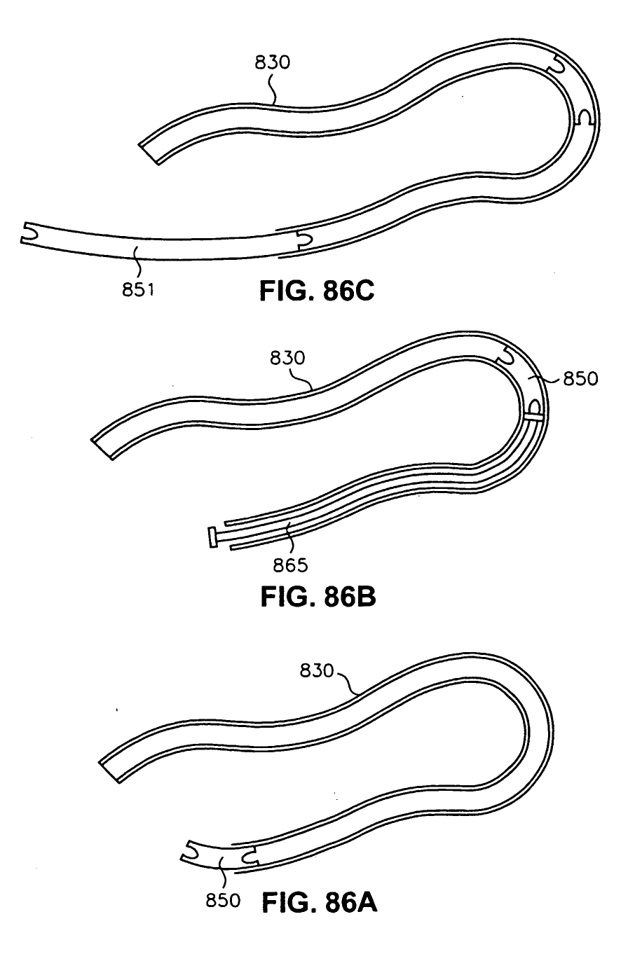

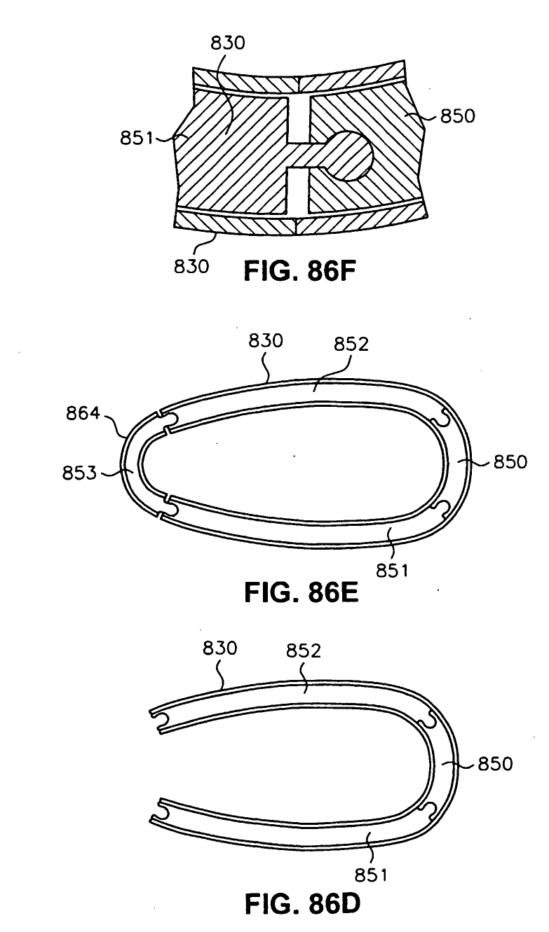

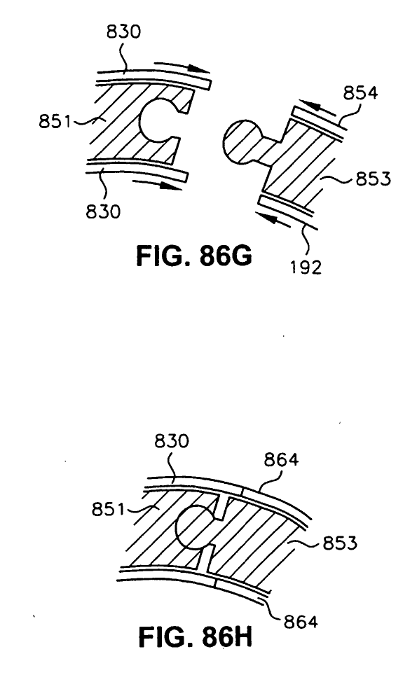

As shown, for example, in FIG. 83, the present invention is also directed to a flexible sheath 830 containing rigid adjustable or non-adjustable mating segments configured to be linked together to form main segment 10. FIGS. 83, 84A, 84B, 85A, [88]85B, [88]85C and 85D illustrate an embodiment of flexible sheath 830 which is placed around natural heart 1 or a portion thereof. Individual segments, for example first, second and third segments 850, 851, and 852, respectively, are then slipped into sheath 830 as shown in FIG. 86A, 86B, and 86C. As discussed more fully below, individual segments 850, 851, and 852 may be flexible, rigid, or semi-rigid and may be interlocking or non-interlocking, depending on the particular remodeling effect desired on natural heart 1 or a portion thereof. Segments 850, 851, and 852 may also be contoured as shown in FIGS. 86A-86C to effect a desired shape change. A fourth segment [854]853 (as shown in FIG. 85D) (which may also be contoured) has its own flexible sheath [854]864. Main segment 10 may be formed from any number of these individual segments.

FIG. 83 shows a flexible sheath 830 in accordance with the present invention. FIGS. 84A and 84B illustrate two views (84A a perspective view, and 84B a sectional view) of natural heart 1 with a flexible sheath 830 adjacent heart 1. FIGS. 85A, 85B, 85C and 85D show a set of rigid segments 850, 851, 852, and 853. These segments are configured to hinge or pivot against each other at ends with lateral stability provided by flexible sheath 830. First, second, third, and fourth segments 850, 851, 852, and 853, respectively, shown in FIGS. 85A, 85B, 85C and 85D may or may not be interlocking. FIGS. 86A, 86B and 86C, however, show a preferred embodiment of first and second segments 850 and 851 in which the segments are interlocking in this example by use of a ball and socket joint. Flexible push rod 865 is used to position the segments within sheath 830. FIG. 86F shows an enlarged cross-sectional view of the final end joining shown in FIG. 86E.

In accordance with principles of the present invention, flexible sheath 830 containing first, second and third segments 850, 851, and 852, respectively, can be assembled as follows. Referring to FIGS. 86A, 86B, 86C, 86D, 86E and 86Ff, first segment 850 (for example, basal segment for placement near basal portion of heart) is inserted into the tube using flexible push rod 865. Next, second segment 851 (for example, an anterior segment) is inserted into flexible sheath 830. Second segment 851 is then click-locked onto first segment 850. Next, third segment 852 (for example, a posterior segment) is inserted into flexible sheath 830 and is then click-locked onto the fist segment 850. Fourth segment 853 (for example, for placement near apical portion of the heart) is then inserted into its own flexible sheath 864 and is snapped into place with second and third segments 851 and 852 as shown in FIGS. 86E, 86G, and 86H such that flexible sheath 864 on fourth segment 853 meets and seals with the flexible sheath 830 on second and third segments 851 and 852.

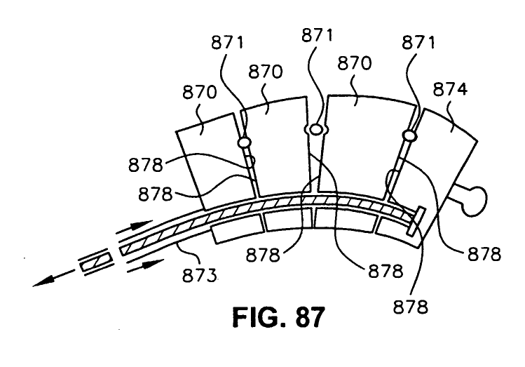

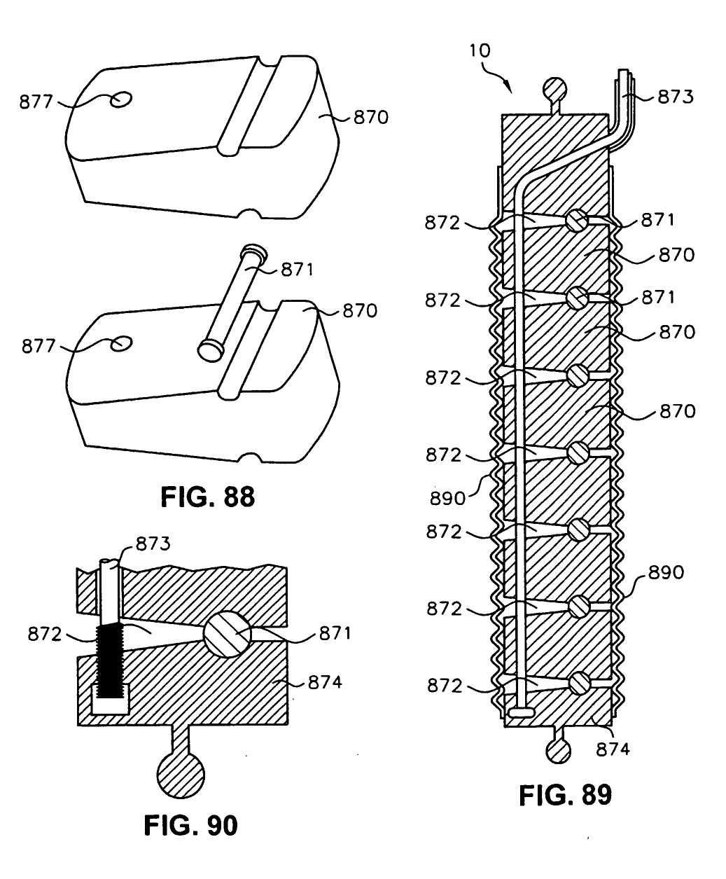

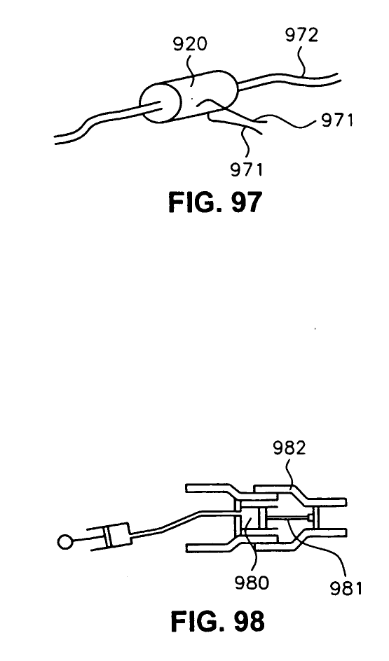

Another aspect of the present invention relates to apparatus and methods for altering die length or curvature of main segment 10. FIG. 87 shows a portion of a segment including a pull-cord version of a chain of hinged block forming, for example, a main segment [10] according to the present invention. As shown in FIG. 87, a series of blocks 870 having pivot pins 871 on one side, tapered edges 878 forming gaps 872 (see FIG. 89) on the opposite side, and a cable, cord or wire 873 attached to one of blocks 874 at one end of main segment 10. When the cable, cord or wire 873 is pulled, the side of the assembly on which blocks 870 have gaps is tightened and individual blocks 870 pivot around pins 871, with gaps 872 closing and blocks 870 coming into contact, thereby shortening that margin and bending the whole segment. Although only four blocks are shown in FIG. 87, any number of many more or less blocks can be used to form the desired length as shown in FIG. 89. As shown in FIG. 87, one of end blocks 874 is a cable-entry block, which is fixed to cable or cord or wire 873. When cable, cord or wire 873 is moved relative to the blocks 870, the other of end blocks 874 containing an end of cable, cord or wire 873 moves relative to the first end block 874 and main segment 10 bends. In one embodiment, one end of cable, cord or wire 873 is threaded into one of end blocks 874, and as a user winds or unwinds cable, cord or wire 873 into one of end blocks 874, one end of main segment 10 moves relative to the other end of main segment 10 and the segment bends. Although described with respect to main segment 10, the structure shown in FIG. 97 can be used for any of segments 850, 851, 852, or 853. FIG. 88 shows one example of two blocks 870 and one pin 871. Holes 877 receive cable, cord or wire 873.

In one embodiment, shown in FIG. 89, main segment 10 has a flexible outer sheath 890 which, for example is corrugated or smooth mesh, as in FIGS. 51B, 51C, 51D, 51E, 69A, 69B, and 83.

Additional mechanisms according to the present invention for adjusting curvature are described below. For example, FIG. 90 shows an embodiment where an end of cable, cord or wire 873 is threaded and is designed to rotate at its end when twisted remotely so as to bring portions of blocks 870 together and close gaps 872. In the embodiment illustrated in FIG. 90, as cable 873 is turned, block 874 is pulled closer to its adjacent block 870, closing gap 872. In turn, all blocks 870 comprising main segment 10 are pulled around their respective pins 871 so as to increase the curvature of the overall segment. In an alternative embodiment, the cable, cord or wire 873 is [be] pulled axially to shorted it and tighten the blocks 870 around their respective pins 871. Alternative embodiments can also achieve the objective of changing the bending moment, or curvature, of a segment according to the present invention, thereby effecting the radius reduction of a chamber of the natural heart.

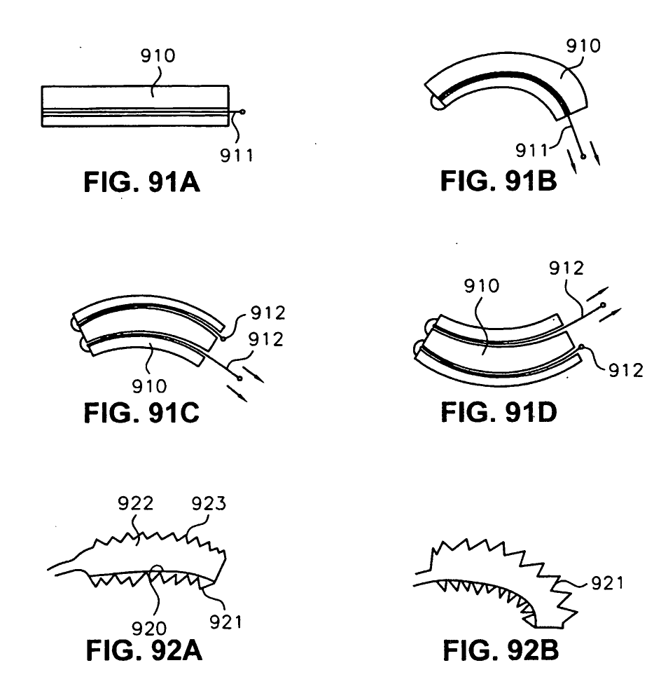

Another such example is illustrated in FIGS. 91A, 91B, [91C and 91D,] wherein a remodeling member in the form of flexible strip 910 has a cable, wire, or cord [911]912 disposed through one side of it. When the cable, cord, or wire 911 is shortened, for example by pulling, strip 910 tightens and curves to the side of the cable, wire, or cord 911, as shown in FIGS. 91A and 91B. FIGS. 91C and 91D illustrate a slightly different embodiment where two cables, cords, or wires 912 are both disposed within strip 910 or adjusting curvature of strip 910. This allows a balancing of forces and easy reopening of strip 910 by pulling on cable, cord, or wire 911 on the side opposite the curvature.

Another embodiment could be used to provide the bending moment discussed above. FIGS. 92A and 92B illustrate the use of hydraulics to achieve the change in bending moment. Flexible segment 920, which is not stretchable in a longitudinal direction, but which is bendable, is connected on its ends to a flexible, corrugated sheath 921 having a cavity 922. The sheath is inflated with a fluid as shown by the arrow in FIG. 92b, and pressure within sheath 921 causes the segment to bend in the direction dictated by flexible segment 920 causing corrugations 923 on upper wall [using upper teeth 925] to expand [and lower teeth 921]while corrugations 921 on lower wall adjacent flexible segment 920 remain near original length[to compress]. As the fluid is allowed to evacuate cavity 922, the [teeth]walls return to their released state and main segment 10 straightens, as shown in FIG. 92A.

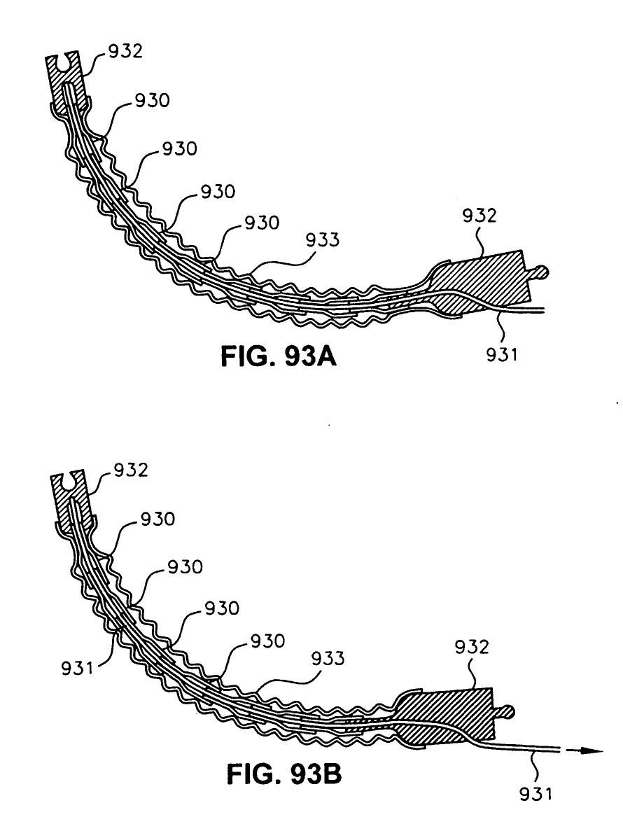

The present invention also provides additional mechanisms and embodiments for modifying the length and/or curvature of main segment 10, thereby effecting the radius reduction of a chamber of the natural heart. For example, FIGS. 93a and 93b illustrate a series of telescoping segments 930 which are narrow at one end and wider and the other, each narrow end being a male end and each wider end being a female end to allow variance in the length of the overall segment. In this embodiment, a cable, cord or wire 931 is run throughout telescoping segments 930. At each end of main segment 10 are ends 932 which for example in this embodiment, have the male and female ball and socket joints as described above for adjoining several segments to each other. Optionally, a sheath 933 also surrounds the telescoping segments 930. FIG. 93B shows the effect of shortening main segment 10, for example by pulling the cable or wire or cord 931.

As described above, various mechanical means may be utilized to shorten cable, cord, or wire 931, such as simply pulling it, or using a threaded torsion end which moves in and out of end 932 as the cable, cord, or wire 931 is rotated. Moreover, any appropriate hydraulic or mechanical means may be used to shorten the overall length of [the]a main segment by taking advantage of the series of telescoping segments 930.

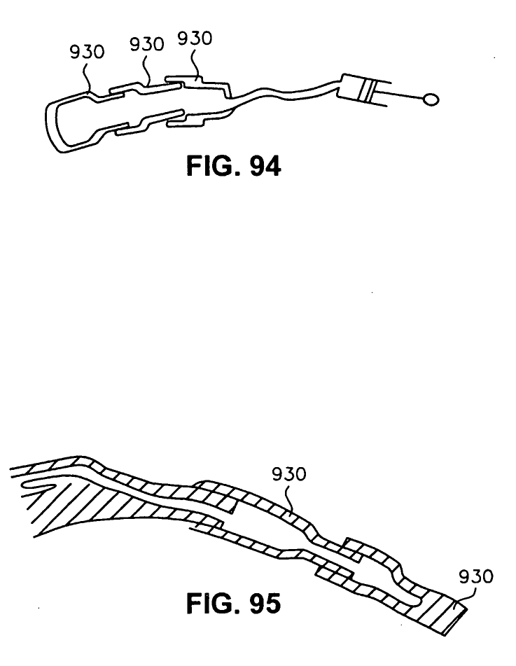

FIGS. 94 and 95 also show the use of a hydraulic system to change the length of a segment according to the present invention comprised of a series of telescoping segments 930. As shown in FIG. 94, as a fluid is pumped into the hollow segments 930, the pressure increases and segments 930 separate, increasing the overall length of a main segment [10]compressing segments 930. FIG. 95 shows a similar embodiment but where the telescoping segments are of a slightly smaller width relative to their length.

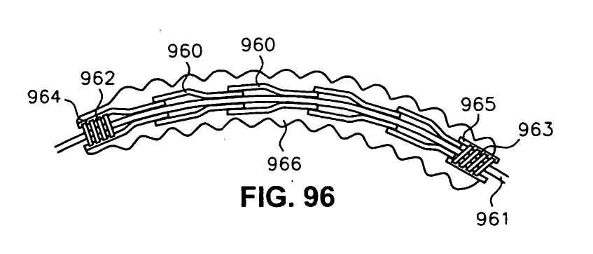

FIG. 96 shows another embodiment useful for adjusting the length of a segment according to the present invention. In this case, telescoping tubular segments 960 are placed over a cable 961. Cable 961 is also fixedly attached to a threaded segments 962 and 963 on each end of a main segment [10]. Each threaded segment 962 and 963 is disposed within an appropriate thread accepting housing 964 and 965 at each end of the main segment [10]. Threaded segments 962 and 963 are disposed opposite each other so that rotation of the cable 961 in one direction causes compression between the two threaded ends. In this embodiment, optionally a sheath 966 surrounds telescoping segments 960.

Cable 961 can be rotated mechanically or electromechanically from a local or remote source. In the case of electromechanical rotation of cable 961, an appropriately geared motor may be used to rotate or torque cable 961 or it can be interposed along the cable itself. In the embodiment is shown in FIG. 97, cable 972 is rotated via motor 970 which is powered and controlled by wires 971. Motor 970 may be within or outside the patient.

In another embodiment, hydraulics similar to those was discussed above, may be used to supply fluid pressure to telescope main segment 10. FIG. 98 shows an embodiment where a hydraulic fluid is used to bias a piston rod rather than filling a telescoping segment as discussed above. In this embodiment, a [piston]cylinder 980 is filled or evacuated which results in the movement of a piston rod 981 outward or inward, respectively, thereby moving telescoping segments 982. Because piston rod 981 is attached to the adjacent telescoping segments, desired movement of the segments is thereby achieved.

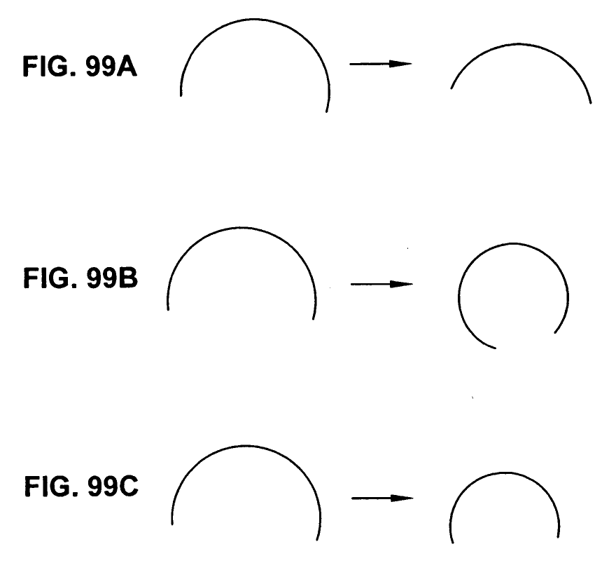

A combined length adjustment and curvature adjustment of one or more of any of the segments according to the present invention can be accomplished by combining the elements as discussed above. This is especially beneficial when trying to adjust both the length and curvature of a main segment [10] so that it properly and completely contacts the individual patient's heart surface, thereby effecting the radius reduction of a chamber of the natural heart. FIGS. 99A, 99B, and 99C show that the elements discussed above can be combined to create, for example, a main segment [10] configured for use adjacent a basal or apical portion of the natural heart. FIG. 99A shows an embodiment where the segment can be adjusted from arc (1) to arc (2) where arc (2) has a lesser length than, lesser angle of curvature than, and the same radius of curvature, as arc (1). FIG. FIG. 99B shows that the segment can be adjusted from arc (1) to arc (2) where arc (2) has the same length as, a greater angle of curvature than, and a lesser radius of curvature than arc (1). FIG. 99C shows that a main segment [10] can, with proper balance of the elements discussed above, be adjusted from arc (1) to arc (2) where arc (2) has a lesser length than, a lesser radius of curvature than, and the same angle of curvature as arc (1).

Assembly for Minimally Invasive Adjustment

The present invention also provides an assembly for minimally invasive position adjustment of the devices of the present invention, including a main segment [10] and/or adjustable stabilizer/reconfiguration segment [12] as described herein, or other devices. The adjustment assembly of the invention can be positioned near the skin surface to which adjustments may be made, for example, by one or more skin-penetrating needles or open exposure through one or more small incisions, and non-invasive or minimally invasive procedures.

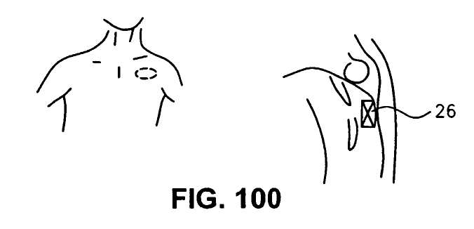

The adjustment assembly can include, for example, a control means, such as control means [1000]26 (such as canister 520 in FIGS. 52-53) illustrated in FIG. 100, that is positioned similar to the position of cardiac pacemakers, percutaneous intravenous infusion ports, or percutaneous dialysis access sites.

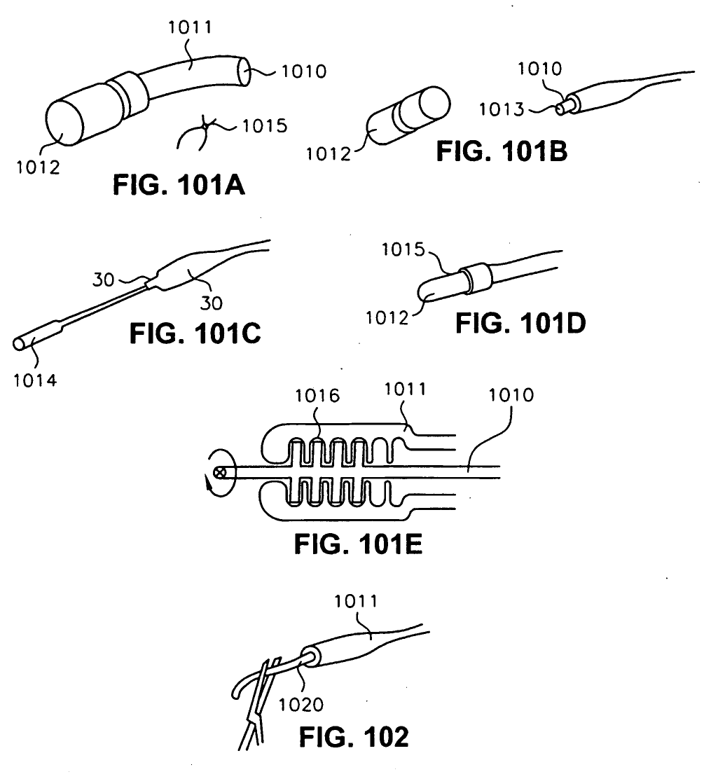

The adjustment assemblies of the present invention can include a coupling and a mechanism internal to the clasp itself to adjust the spacing between two main segments [10], such as those shown in FIGS. 101A, 101B, 101C, 101D, 101E, 102, 103, 104, and 105A-114B. The coupling is positioned between the superficial mechanism and the mechanism internal to the clasp. The clasp internal mechanism is located within or upon one or more components of main segment 10 which responds to superficial mechanism adjustment by effecting a change in the relative position of the heart-contacting surfaces of two or more main segments [10] related to one another, of some portion or portions of a main segment [10], and/or of the adjustable stabilizer/reconfiguration segments [12].

An embodiment of an adjustmnent assembly of the invention is illustrated in FIGS. 10A, 101B, 101C, 101D and 101E. In this embodiment, rotation of a cable 1010 effects a change in the position of a main segment [10] and/or an adjustable stabilizer/reconfiguration segment [12]. As shown in FIG. 101A, cable 1010, such as a cable, cord, wire, is located within a casing 1012 and is attached to a main segment [10] and/or adjustable stabilizer/reconfiguration segment [12] (not illustrated). A tip 1013 (shown in FIG. 101B) of cable 1010 is covered by cap 1012 that is removably connected to the casing 1011 covering cable 1010. Cap 1012 can be removably connected to the casing 1011 using conventional means, such as a pressure fit, suturing, and the like.

As shown in FIGS. 101B and 101C, cap 1012 can be disconnected from casing 1011 such that a tip 1013 of cable 1010 is exposed. In the embodiment shown in FIG. 101B, a pressure clip 1015 is removed from cap 1012. Tip 1013 can then be rotated using an instrument 1014, such as screwdriver or allen wrench, to turn cable 1010. Rotation of cable 1010 effects a change in the relative position of the heart-contacting surfaces of two or more main segment [10] bars, of some portions of main segments [10], and/or of the adjustable stabilizer/reconfiguration segment [12.] Following adjustment of main segment [10] and/or adjustable stabilizer/reconfiguration segments [12], the cap 1012 can be reconnected to the casing, as shown in FIG. 101d. FIG. 101e illustrates an exemplary screw mechanism 1016 by which[for] rotating cable 1010 within casing 1011 effects linear translation of cable 1010 within casing 1011.

FIG. 102 illustrates another embodiment of an adjustment assembly of the present invention. The adjustment assembly illustrated in FIG. 102 includes a direct push-pull-driven linearly moving cable 1020 surrounded by a casing 1011. Cable 1020 illustrated in FIG. 102 can include a removable cap, such as the removable cap 1012 illustrated in FIGS. 101A, 101B, 101C, 101D, and 101E. A push or pull movement of cable 1020 within casing 1011 causes a change in the relative position of the heart-contacting surfaces of two or more main segments [10], of some portion or portions of main segments [10], and/or of adjustable stabilizer/reconfiguration segments [12]. The position of cable 1020 can be locked after adjustment by a set-screw, a knot, and the like (not shown).

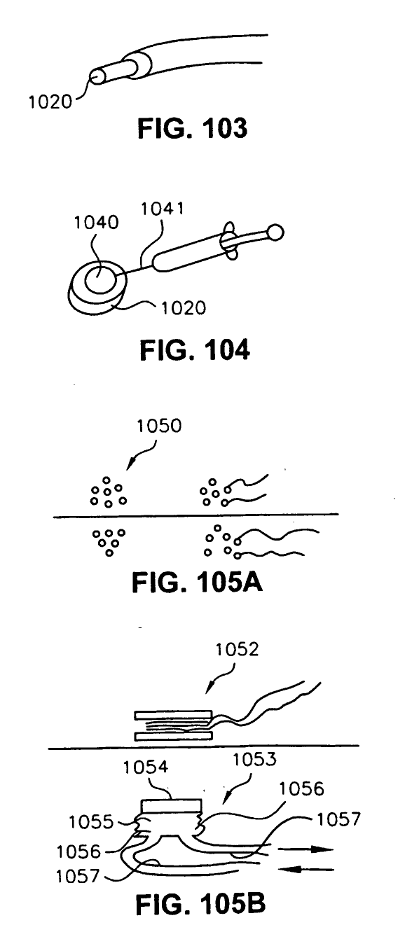

FIG. 103 illustrates another embodiment of an adjustment assembly of the present invention. Cable 1020 illustrated in FIG. 103 is similar to cable 1020 illustrated in FIG. 102, but is shaped to permit rotation by hand and without the use of an instrument.

FIG. 104 provides another embodiment of an adjustment assembly of the present invention. As shown in FIG. 104, cable 1020 can include a port 1040 for receiving a fluid. A needle 1041 may be inserted either percutaneously or after exposure through an incision for supplying and/or withdrawing fluid through port 1040 and into or out of cable 1020. If an incision is made, the needle 1041 and penetrable diaphragm may be replaced by a stopcock and mating tube-ends.

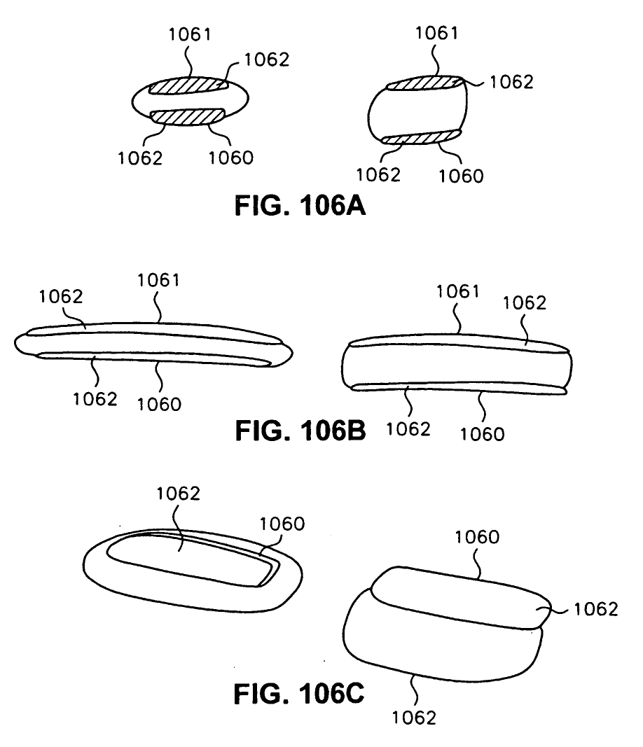

Another embodiment of an adjustment assembly of the present invention is illustrated in FIGS. 105A and 105B. As shown in FIGS. 105A and 105B, an electric or magnetic mechanism 1050 is driven by a transcutaneous coupling 1051. FIG. 105a shows an electrical transformer 1050 similar to the Transcutaneous Energy Transfer System (TETS) used for driving circulatory support. FIG. 105B shows a solenoid/permanent magnet 1052 [driven by]driving a hydraulic pump 1053. In one embodiment, replacement of the passive valves by magnetically reversible one-way valves would allow reversal of flow if desired. In one exemplary embodiment, pump 1053 includes a passive or active magnetic armature 1054, a hydraulic bellows 1055, valves 1056, and tubes 1057. The relative spacing of a main segment [10] and adjustable stabilizer/reconfiguration segment [10] can be adjusted by similar movement or electrical rotation of elements, for example, in any of FIGS. 87, 88, 89, 90, 91A, 91B, 91C, 91D, 92A, 92B, 93A, 93B, 94, 95, 96, 97, 98, 101A, 101B, 101C, 101D, 101E, using embodiments shown in FIGS. 103, 104, 105A and 105B.

Main segment [10] and/or adjustable stabilizer/reconfiguration segments [12] of the present invention can include a movable inner surface 1060 that is positioned adjacent the heart, and an outer surface 1061 opposite movable inner surface 1060 that does not contact the heart. FIGS. 106a-113b illustrate embodiments of the invention for movement of an by inner (heart-contacting) surface 1060 of a main segment [10] and/or adjustable stabilizing/reconfiguration segments [12] relative to an outer non-heart contacting surface 1061 of the main segment [10].

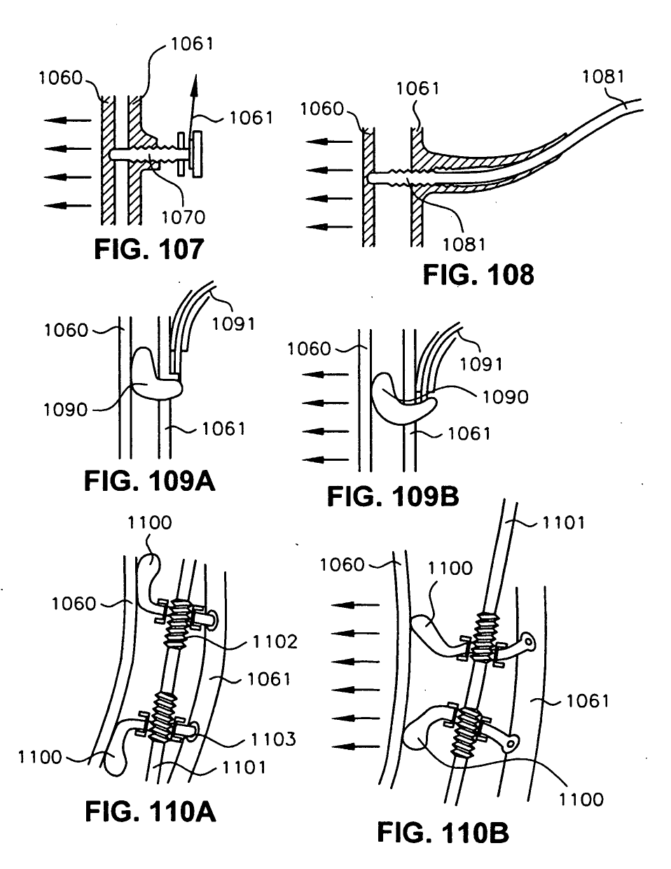

FIGS. 106A, 106B, and 106C illustrate an optional conforming jacket 1062 that can be employed in any of the mechanisms illustrated in FIGS. 107-113B. The conforming jacket illustrated in FIGS. 106A, 106B, and-106C is shown (a) cross-sectional view, (b) long sectional view, (c) perspective external view, respectively.

FIG. 107 illustrates a screw-operated pusher 1070 driven by a pull-cord 1071 for movement of the inner (heart-contacting) surface 1060 relative to outer surface 1061. FIG. 108 illustrates a screw-operated pusher 1080 driven by a torque-cable 1081 for movement of the inner (heart-contacting) surface 1060 relative to outer surface 1061. FIGS. 109A and 109B illustrate a [screw-operated] lever 1090 operated by a pull cord 1091 for movement of the inner (heart-contacting) surface 1060 relative to outer surface 1061.

FIG. 110A and 110B illustrate a screw-operated lever 1100 operated by a torque-cable 1101 for movement of the inner (heart-contacting) surface 1060 relative to outer surface 1061. When cable 1101 is rotated, threaded segments 1101 and 1103 cause levers 1100 to come toward each other which results in the separation of surfaces 1060 and 1061 as shown in FIG. 110b.

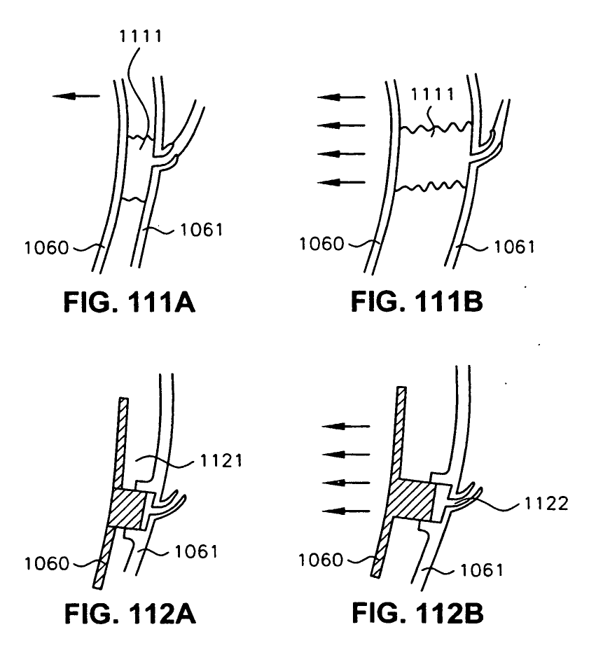

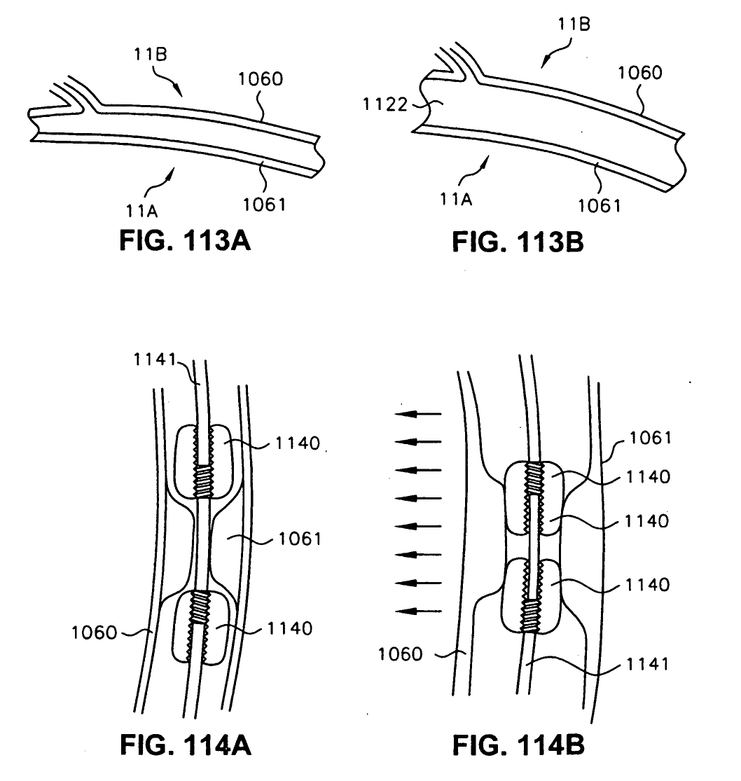

FIGS. 111A and 111B illustrate a hydraulic bellows 1111 for movement of the inner (heart-contacting) surface 1060 relative to outer surface 1061. FIGS. 112A and 112B illustrate a hydraulic piston 1121 for movement of the inner (heart-contacting) surface 1060 relative to outer surface 1061. In another embodiments inner surface 1060 is moved relative to outer surface 1061 via a direct hydraulic space 1122 between inner and outer surfaces 1060 and 1061, respectively is illustrated in FIGS. 113A and 113B. FIGS. 114A and 114B illustrate screw-approximating shims 1140 for movement of the inner (heart-contacting) surface 1060 relative to outer surface 1061. Here, shims 1140 are moved toward each other as the cable 1141 is rotated. This causes the separation of the inner (heart-contacting) surface 1060 relative to outer surface 1061.

As discussed above relative to FIGS. 37A and 37B, the present invention can also include an apical cap (or bowl-shaped device) that fits over the outer (epicardial) surface of the apical part of the left ventricle for stabilizing devices adjacent heart 1. Such an apical cap may or may not extend onto the apical portion of the right ventricle. This aspect was discussed briefly above in regard to FIGS. 37a and 37b which illustrate an embodiment of an apical coupling 370, such as a mounting block or cap, that fixes two or more reconfiguring bundles 320 of spring wires 321 together. Such an apical cap can also be used to stabilizing main segment 10 on the heart.

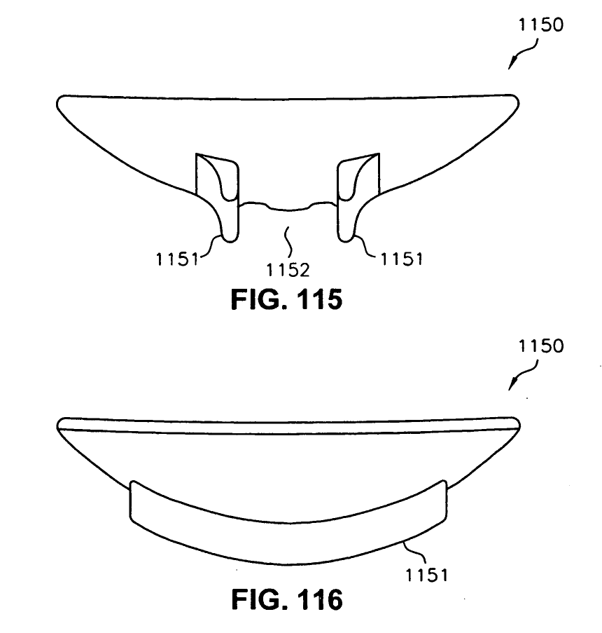

As shown in FIG. 115, apical cap 1150 (e.g., coupling 370 described with respect to FIGS. 37A and 37B) has a shape and stiffness, particularly in the radial direction, which will not allow it to move substantially in any direction perpendicular to the long axis of the left ventricle. It provides, therefore, a stable anchoring member to prevent motion of a device on or in the heart surface, such as a main segment [10] or bundle 320 of springs 321.

As shown in FIGS. 115 and 116, apical cap 1150 is designed to fit adjacent the apical part of the left ventricle. Two or more protrusions 1151 form a channel 1152 which is deep enough to receive main segment 10. FIG. 116 is a side view of apical cap 1150 shown in FIG. 115. In one embodiment, apical cap 1150 is made from a relatively soft material, preferably one having at least a durometer hardness Shore A of 60.

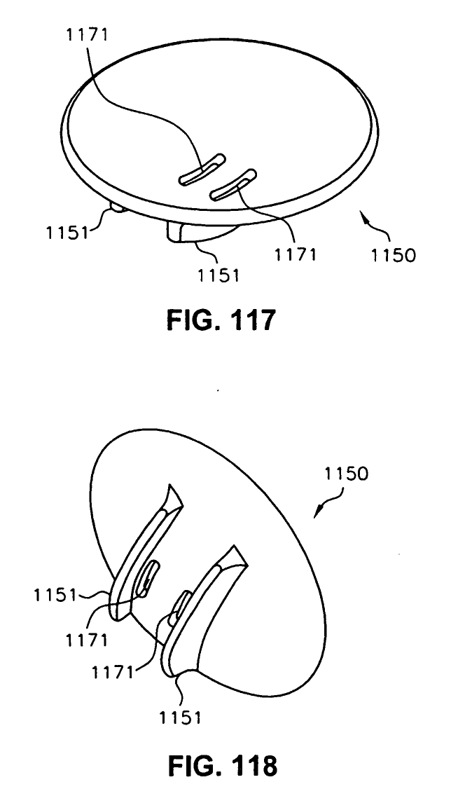

FIGS. 117 and 118 show isometric views of apical cap 1150. FIG. 117 also shows two suture slots or holes 1171. Slots 1171 are used to suture the apical cap [1157]1150 to the heart. Alternatively, or in addition to receiving sutures, slots 1171 can also perform the function of the coupling holes for receiving post tip 330 described above with respect to FIG. 37A.

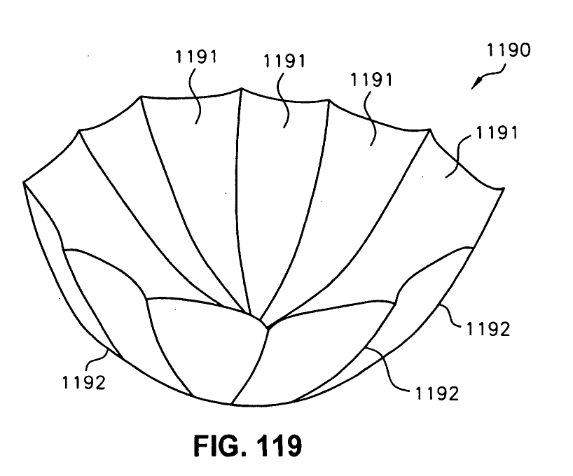

FIG. 119 is a perspective view of another embodiment of an apical cap 1190. Apical cap 1190 is made of multiple (generally 12 or more) panels 1191 of soft biocompatible fabric which have been sewn or otherwise connected in the form of a “beanie.” Panels 1191 are joined, in this particular drawing, at seams 1192. Seams 1192 perform the additional function of adding controllable stiffness in the radial direction, which prevents wadding or folding in the circumferential direction. Such wadding or folding is not desired because it would enable epical cap 1190 to slip laterally off the apical portion of the heart.

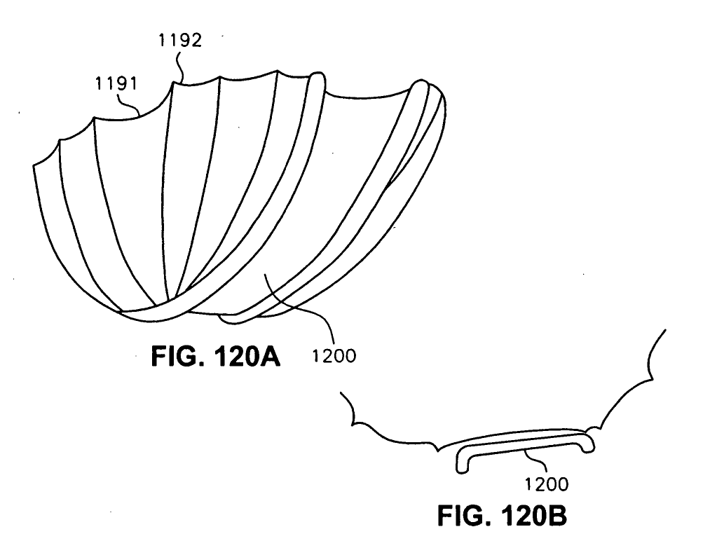

FIGS. 120A and 120B show apical cap 1190 with the addition of a soft polymer guide 1200 (e.g., channel) which facilitates position maintenance for a reconfiguration device such as that shown in FIGS. 2B, 3, 14A, 14B, 53, 55, 63A, 63B, 64A, 64B, etc., including a main segment 10. FIG. 120B is a sectional view of the guide 1200.

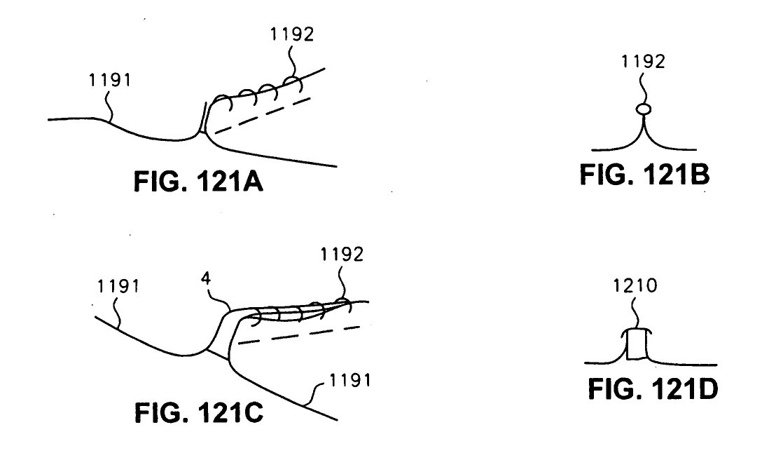

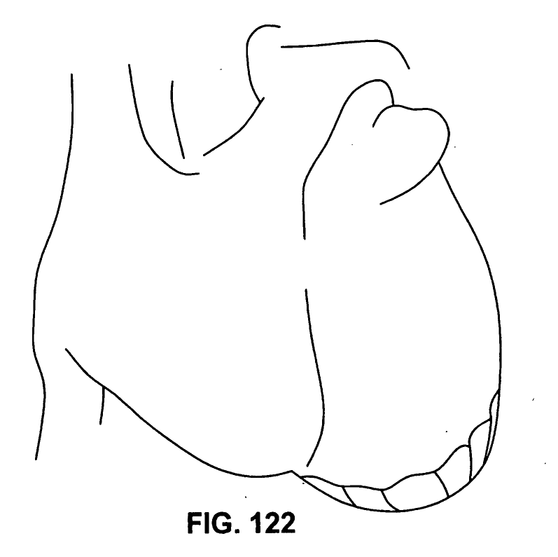

FIGS. 121A, 121B, 121C and 121D show more detail of the seam construction in FIG. 119. FIG. 121a illustrates a simple seam and FIG. 121b a section of that same seam. FIG. 121c is a buttressed seam incorporating a stiffening strip 1210 of additional fabric of felt or other stiffening material in a manner known to those skilled in the art of sewing, and FIG. 121d is a section of that shown in FIG. 121c.

FIG. 122 is a perspective view of an apical cap 1190 placed on a heart in accordance with one embodiment of this part of the invention.

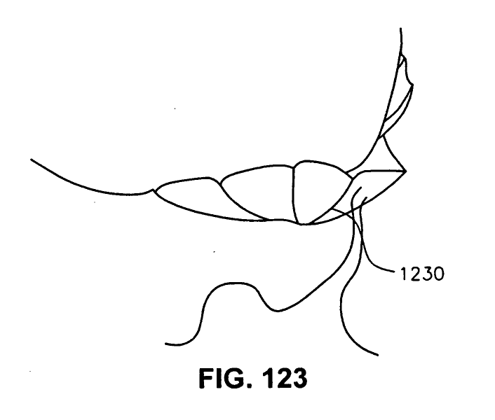

FIG. 123 is a side perspective view of the heart shown in FIG. 122, and also shows a pleat or tuck 1230 provided for circumferential size adjustment of apical cap 1190 using one or more sutures to adjust size.

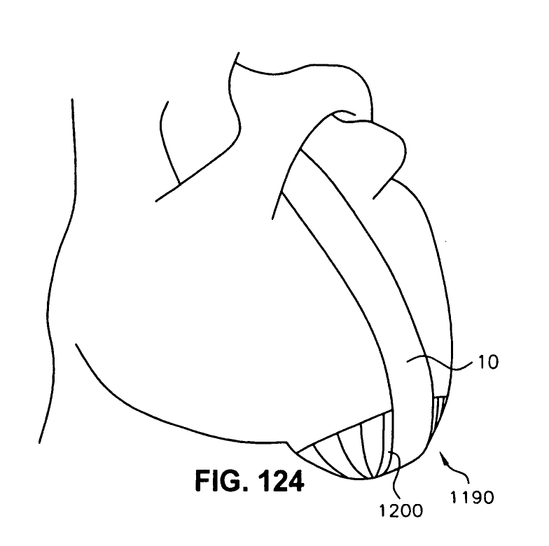

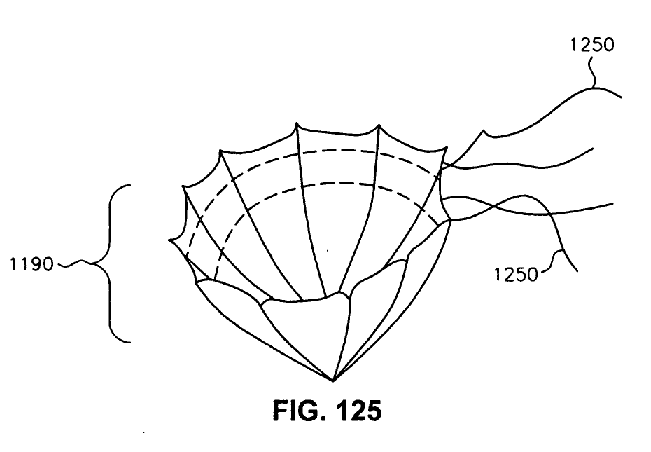

FIG. 124 shows a main segment 10 of a heart remodeling device according to the present invention positioned on the heart, with main segment 10 positioned in guide 1200 of apical cap 1190.

FIG. 125 illustrates apical cap 1190 with circumferential purse strings 1250 entered around one or more portions of apical cap [1180]1190, that may be used to adjust the shape and size of apical cap 1190 as described with respect to FIG. 123. [Four]Two such purse strings, each with two ends, are shown in FIG. 125, but any number may be used. As discussed with respect to FIGS. 69A-72 above, apical cap 1190 may include pacing leads or transceiver elements such as those on main segment 10 or stabilizer/reconfiguration segment 12.

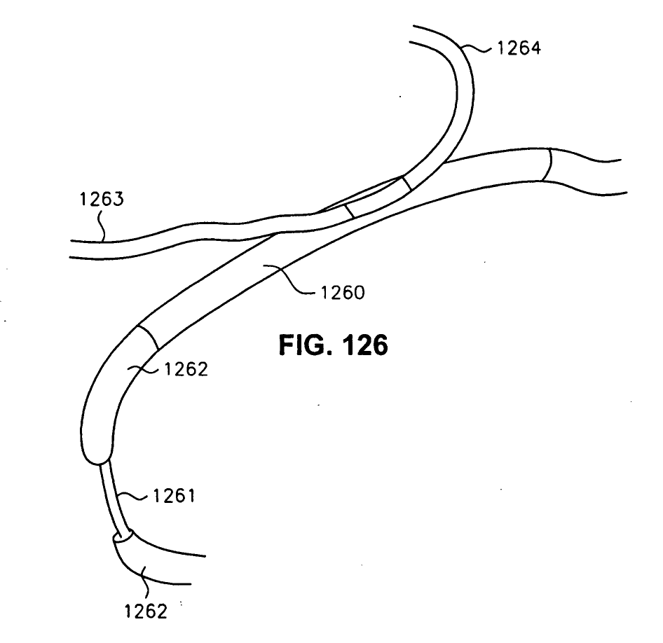

FIG. 126 shows an embodiment for releasably securing cable 481 (e.g., as shown in FIG. 52), to a main segment [10] having a center modular portion 1260, using a remote cable-clamping mechanism. Such a configuration is used to facilitate the general scheme of tether, cable, cord or wire-mounted clasp members by providing ease of placement and remote adjustability, while eliminating the reduction of positional stability inherent in long tethers, cables, cords, or wires disposed within sheaths. It should be noted that when the word “cable” is used, it is intended to be synonymous with the words, tether, cable, cord, wire, chain, strap, or other similar restraining device.

The general principle of this aspect of the invention is that of a cable-car clamp or a detachable ski-lift clamp. The resting position of the spring-activated clamp or brake is closed, so as to prevent cable movement. An active maneuver is required to effect spring release. Thus, the failure mode would presumably be loss of adjustability, as opposed to loss of cable stability.

In one embodiment, the mechanism is a fixation device located on a main segment [10], that can be released and adjusted remotely by an adjustment cable or other means. The clamp-releasing cable itself is different from the cable or tether that was described above with respect to FIG. 52 with regard to the clasp placement system and adjustment. When the cable clamp is released, transiently, by means of this alternate type of cable, the primary (clasp-supporting) cable may be adjusted in length. When the clamp is re-tightened, the primary cable length is again fixed.

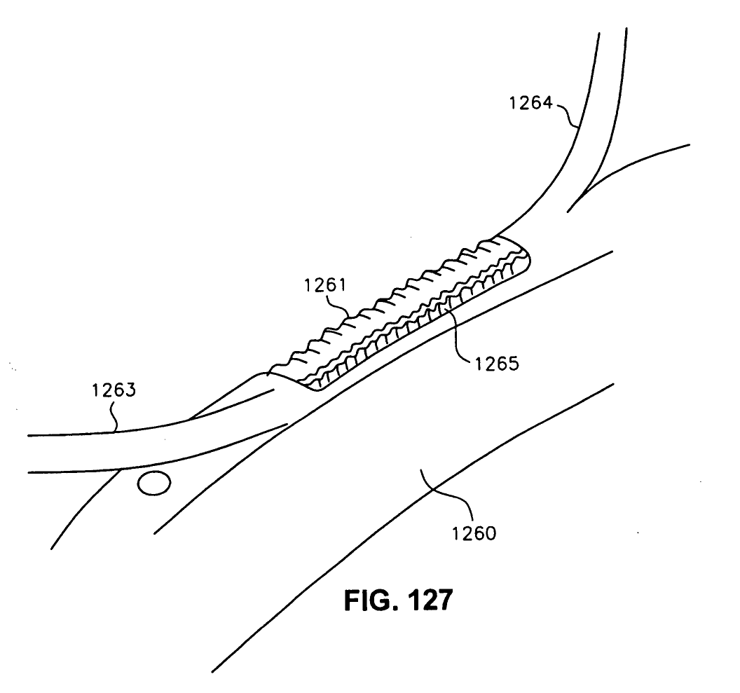

In an embodiment shown in FIG. 126, a main segment [10] is shown with an apical cable 1261 partially exposed as it passes through apical segment 1262 of the spine of the main segment [10]. Sheath 1263 covers an atrial cable 1265 ([not shown, but]illustrated in FIG. 127, and identical to apical cable 1261) and sheath 1264 covers apical cable 1261. It is the cables within sheaths 1263 and 1264 which can control the compression of the main segment [10], as described in more detail below. Cables 1261 and 1265 may be the ends of one cable or two or more cables linked together, for example liked by one or more portions of main elements [10].

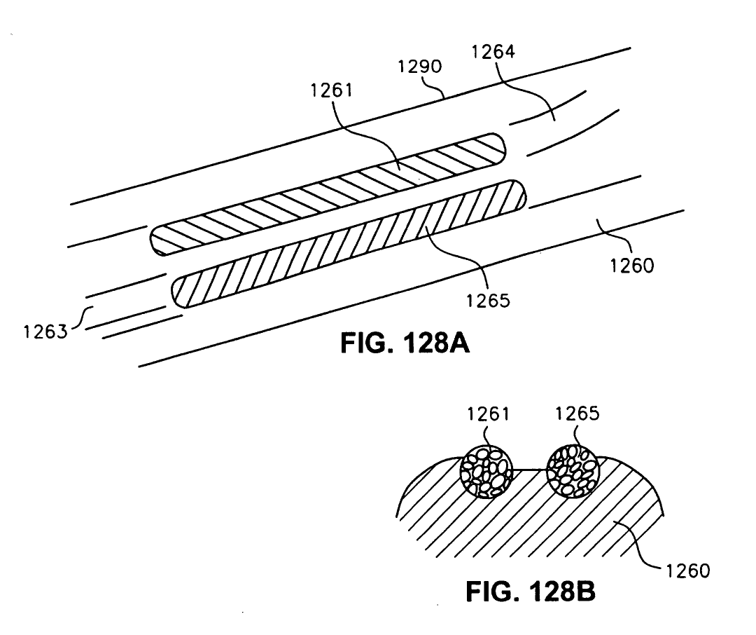

FIG. 127 is an enlarged view of the center part of the main segment [10] shown in FIG. 126. FIG. 127 shows the alignment of sheath 1264 for an atrial cable 1265, sheath 1263 for an apical cable 1261. FIG. 128A is a top view of that shown in FIG. 127. FIG. 128b is a longitudinal cross sectional view along line 128b-128b of a that shown in FIG. 128a.

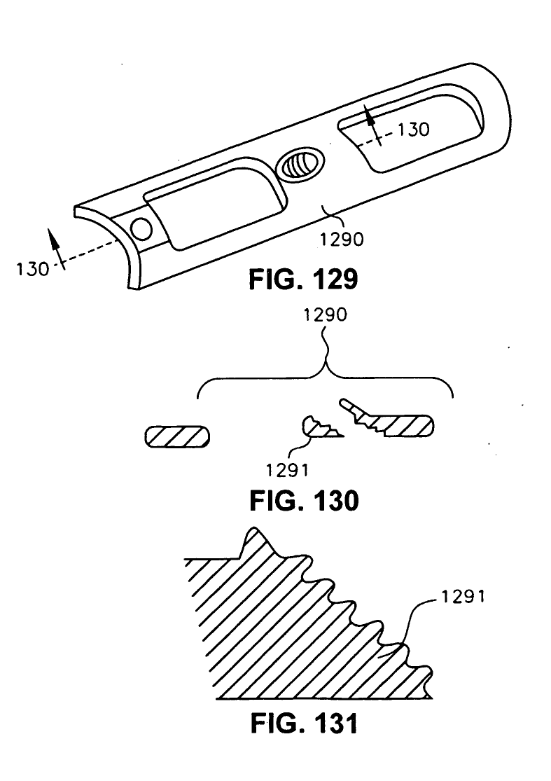

FIGS. 129-131 show the clamping mechanism comprised in the embodiments shown in FIGS. 126-128b. FIG. 129 shows a clamping spring 1290 for clamping cables 1261 and 1265 to a main segment [10]. FIG. 130 shows a longitudinal section through the midline 130-130 of FIG. 129. FIG. 131 shows an enlargement of the threaded hole 1291 of FIG. 130.

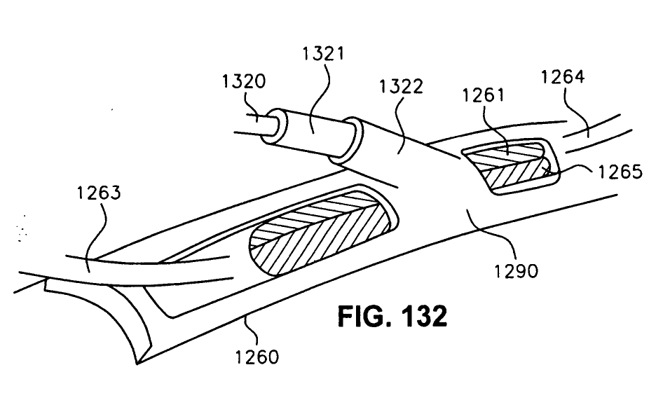

FIG. 132 shows the clamping spring 1290 in position on center modular portion 1260. Cables 1261 and 1265 which hold main segments to each other and on the heart are shown in place, running through modular center portion 1260. [Clamp]Clamping spring 1290[, which] houses clamp releasing cable 1320 which is disposed within cable sheath 1321 and cable[clamp] releasing cable port 1322. More specifically, FIG. 132 shows a perspective view of an embodiment where the pressure and texture of the center cross-bar of the [clamp]clamping spring 1290 imposes a generally normal force on cables 1261 and 1265 such that friction prevents movement of the cables 1261 and 1265 unless a displacing tension in the cables is substantially greater than would arise from conceivable normal physiologic events.

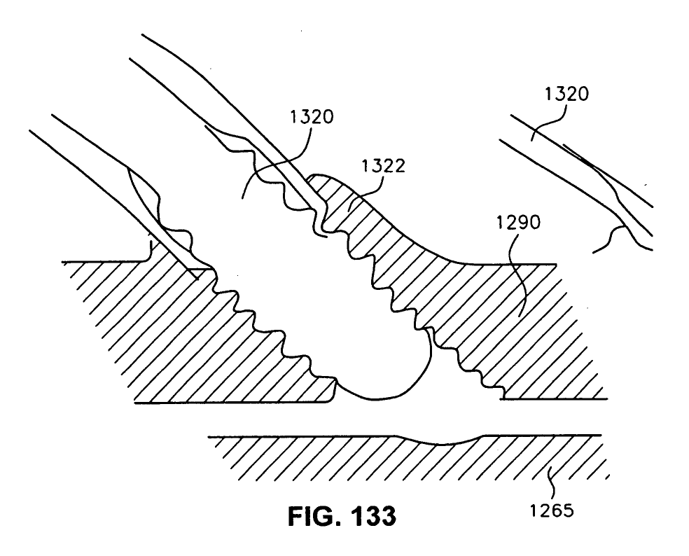

FIG. 133 shows an enlarged view of the clamp releasing cable 1320 and clamp releasing port 1322. Here, torque is applied remotely to rotate clamp releasing cable 1320 which causes the threaded cable to advance into the [clamp]clamping spring 1290, thereby progressively impinging on spine segment [1265]1266. This produces a bending outward of [clamp]clamping spring 1290 so as to separate the [clamp]clamping spring 1290 from spine segment [1265]1266 sufficient to allow cables 1261 and 1265 to move. Cable 1261 and 1265 are resecured to main segment 10 by moving clamp releasing cable 1320 in an opposite direction allowing [claim]clamping spring 1290 to reseat on cable 1261 and 1265.

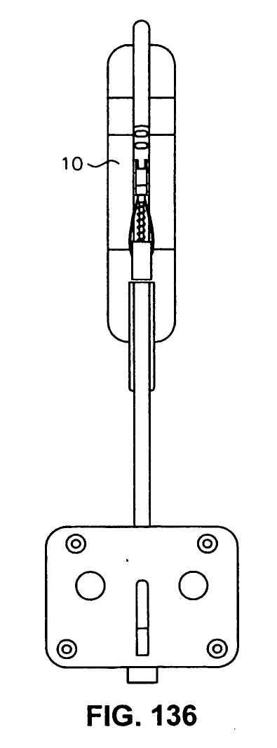

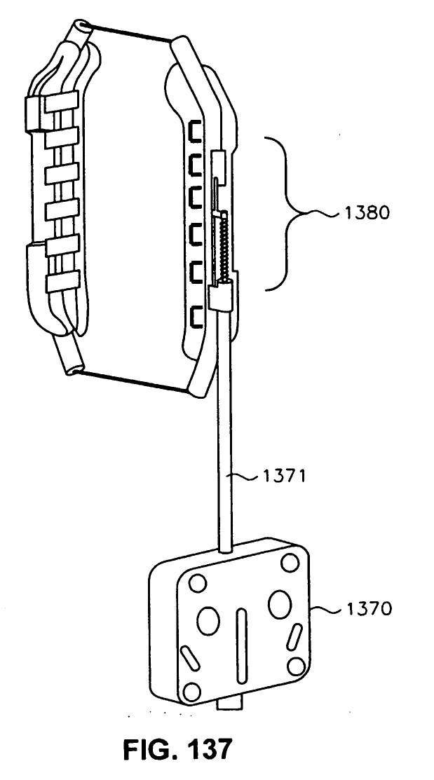

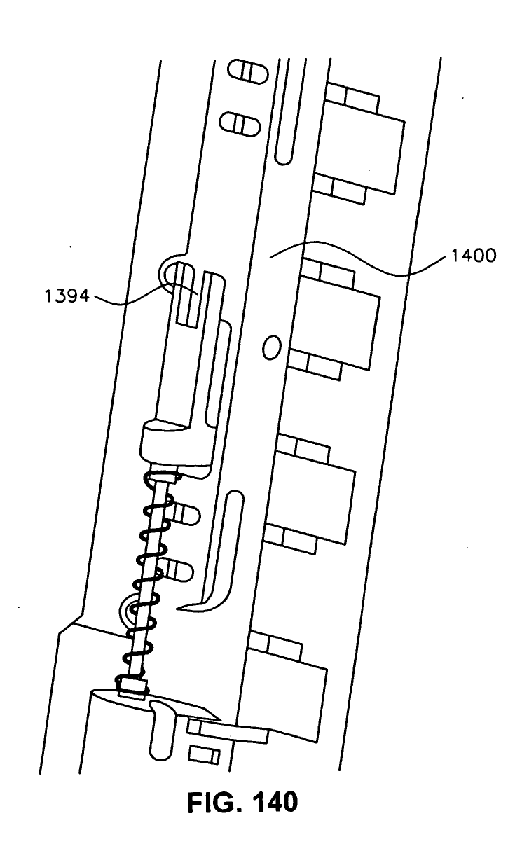

An additional embodiment for releasably locking cables such as cables 1261 and 1265 to main segment 10 is shown in FIGS. 136, 137, 138, 139, 140, and 143.

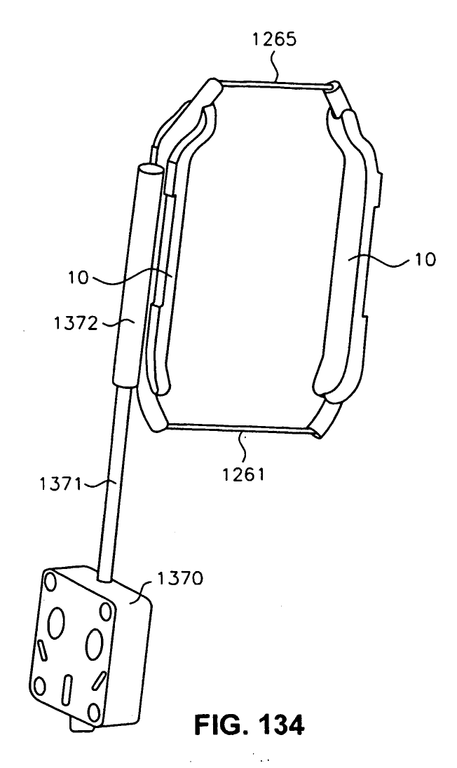

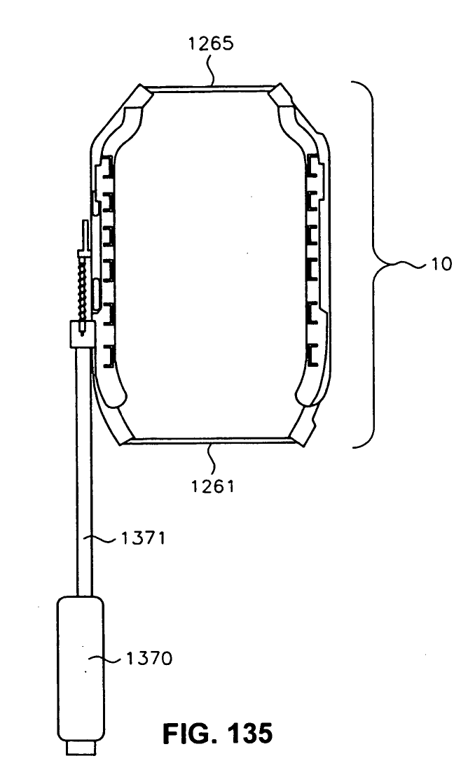

FIGS. 134-137 show side, perspective and isometric views of an alternative locking mechanism 1372. Control box 1370 is shown only to represent that a mechanism for control locking mechanism 1372 is attached thereto and required for releasing a clamp securing cable 1261 and 1265, and optionally, for increasing and decreasing the space between two main segments 10. An umbilical-like connection 1371 connects control box 1370 with the locking mechanism 1372.

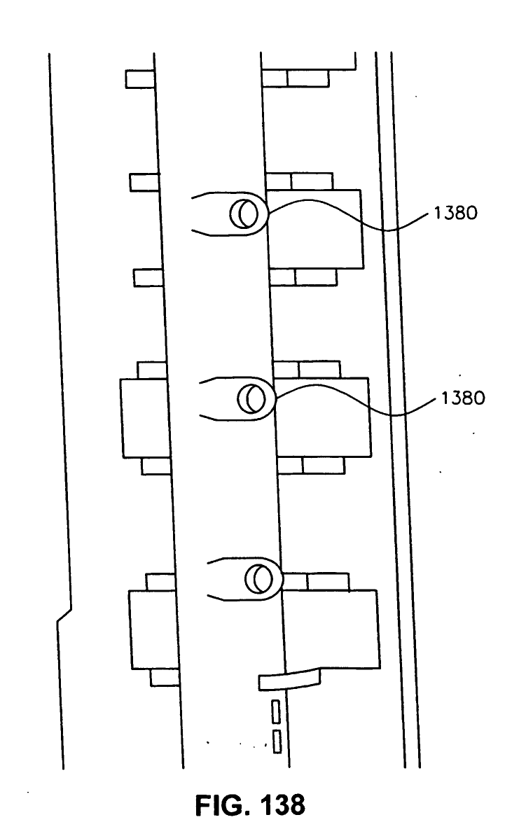

FIG. 138 shows an enlarged view of a portion of [locking mechanism 1372]main segment 10 showing purse string attachment points 1380, as discussed above with respect to a stabilizer/reconfiguration segment 12 in FIGS. 7, 8, 10A and 10B.

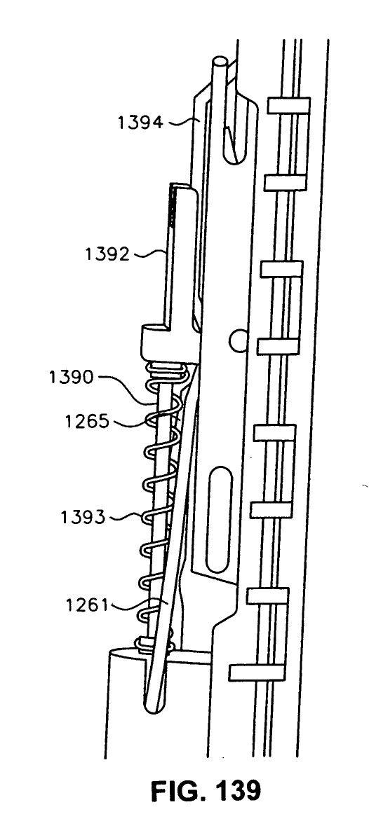

Locking mechanism 1372 shown in FIG. 139 includes cables 1261 and 1265 which pass from umbilical-like connection 1371 into locking mechanism 1372, control cable 1390, spring 1393, and locking wedge 1392. In one embodiment, length of cables 1261 and 1265 is controlled through a ratcheted spool mechanism contained in a control box 1370.

The proximal end of the control cable 1390 is fixed to the control box and the distal end is fixed to the spring loaded locking wedge 1392. Locking mechanism 1372 is composed of locking wedge 1392 and spring 1393, as well as a wedging surface 1394, which is integral with the device frame. A wedging surface 1394 of locking wedge 1392 creates a pinch point for cables 1261 and 1265 between the wedging surface 1394 and a wedge 1400 itself. Wedge 1400 is spring loaded to insure the system will be locked when in the default position. The user can control the locking system through control cable 1390, which passes through umbilical sheath 1371. When the locking system is in the unlocked position, the cables 1261 and 1265 are be tightened or loosened thereby decreasing or increasing the space between two main segments 10. The control box controls cable length and cable tension.

In use, as control cable 1390 is rotated, spring 1393 is compressed and releases pressure on locking wedge 1392 which allows cables 1261 and 1265 to be tightened or loosened. To again secure cables 1261 and 1265 to wedging surface 1394, control cable 1390 is rotated in a opposite direction to decompress spring 1393.

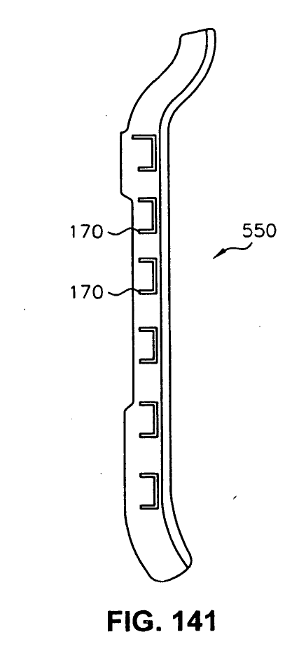

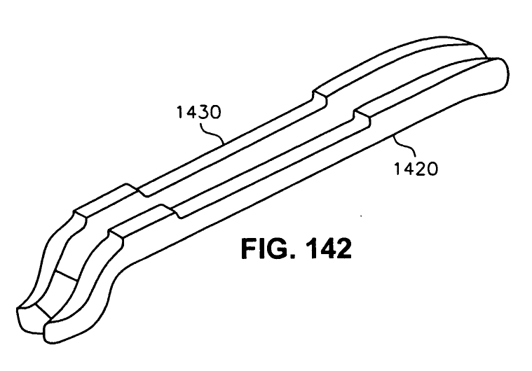

FIGS. 141 and 142 show an additional embodiment of the pad 1430 (which may be, for example pad 550 as described with respect to FIG. 55). Pad [550]1430 has a hardness of 40 to 60 Shore A, and preferably is formed from a polyurethane rubber or implantable grade silicone. The longitudinal radius of curvature 1420 of pad 1430 as shown in FIG. 142 is designed to insure enough curvature to effect the desired shape change of a heart or chamber thereof. For example, the longitudinal radius of curvature of main segment 10 can range from convex to concave toward the heart and can be in the range of minus 120 mm to positive 120 mm.

The radius of curvature of the lateral edges of main segment 10 or plates 170 (as described above) have a radius of curvature in the range of 0.2 mm to 10 mm so the edges do not impact negatively on the heart surface.

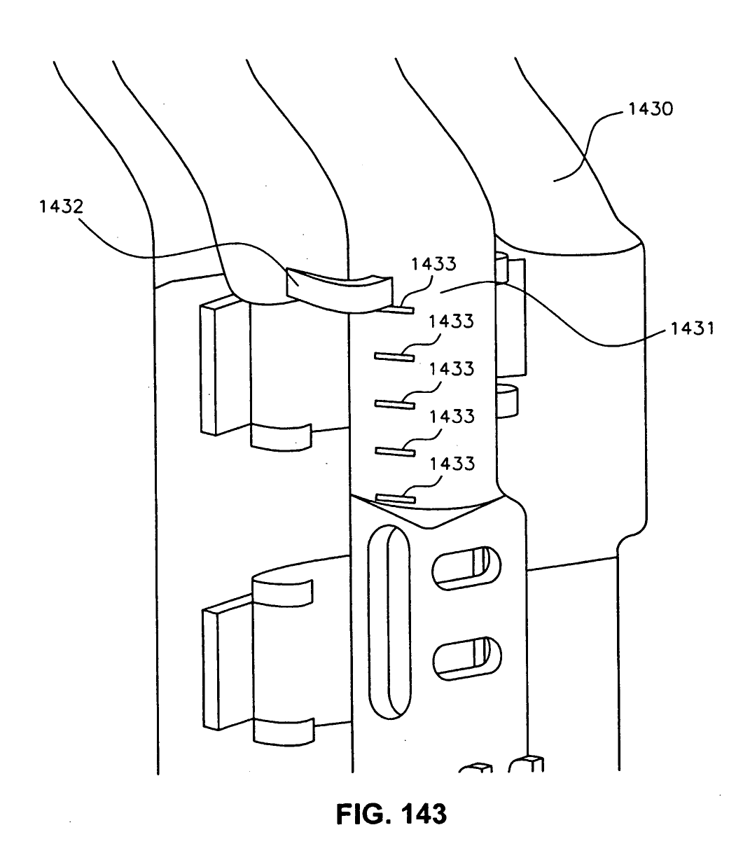

FIG. 143 shows an enlarged view of pad 1430 included in a main segment [10]. Snap-on attachment 1432 holds pad 1430 on bar 1431 of the main segment [10]. Grooves 1433 in [main segment 10]bar 1431 allow about +/−10 degrees of rotation in either direction (overall rotation of about 20 degrees) of the pad 1430. A plurality of grooves 1433 allows the user choices in actual attachment placement to improve the fit to the atrium and atrioventricular groove. Such a plurality should be sufficient to allow placement up or down about 1.5 to 2.5 mm (about 3 to 5 mm overall).

Additional embodiment of the present invention relates to spatial stabilization of a heart geometric remodeling device similar to those disclosed above with respect to FIGS. 7-11B and 55-67. The addition stabilization, structures and uses thereof are described below.

In one embodiment, a strap or band extends from an anterior remodeling segment, in the region of the anterior atrioventricular junction, around the junction of the lateral free walls of the left atrium and left ventricle.

In a second embodiment, a strap or band similar to the above extends from an anterior remodeling segment around the remainder of the left atrium/left ventricular (LA/LV) junction anteriorly, around the entire junction of the right atrial and right ventricular free walls externally, and across the medial-most part of the posterior LA/LV junction to join a posterior remodeling segment. In one embodiment, in a first part of the path of the band or strap, the strap or band passes between the anterior aspects of the atrioventricular junction and the posterior aspects of the aortic and pulmonary artery roots.

A third embodiment relates to a circumferential strap or band placed, for example, around the root of the aorta above the level of the valve commisures and the supravalvular sinuses, and tethered to an anterior remodeling segment by a linear cord or band.

In all three of the above embodiments, minimally invasive placement techniques and remote (including video assisted) assembly are used.

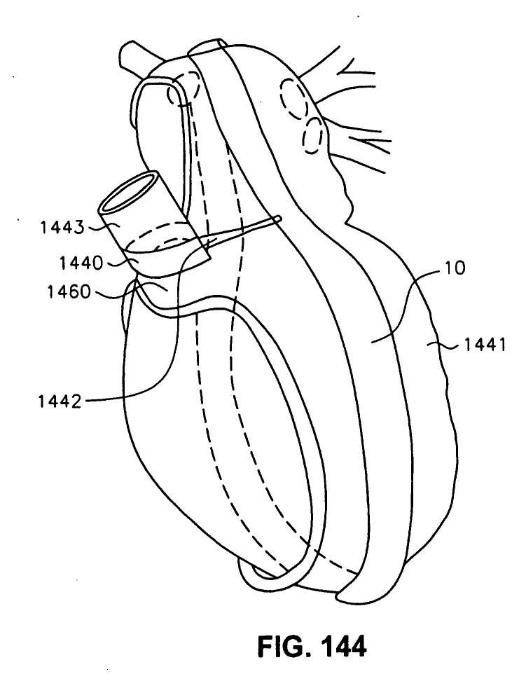

FIG. 144 illustrates an embodiment showing a heart 1 having a device according to one aspect of the present invention. Heart 1 shown in this drawing has had the right atrial and ventricular free walls and the pulmonary artery removed. FIG. 144 shows a main segment 10 encircling a left ventricle 1441 connected via tether 1442 to aortic collar 1440 which surrounds artery 1443.

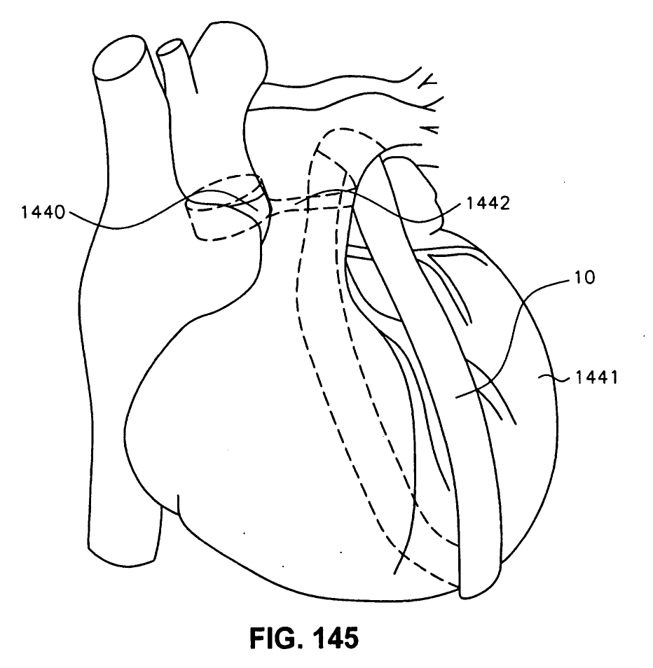

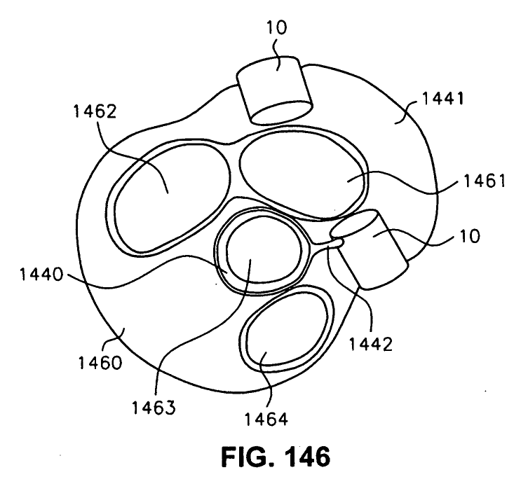

FIG. 145 illustrates the same configuration as that shown in FIG. 144 but without removal of the right atrial and ventricular free walls and pulmonary artery. FIG. 145 shows that tether 1442 passes between the aorta and the atrioventricular junctions, and that collar 1440 may lie partially behind the right atrial appendage.

FIG. 146 shows a top view partial cross-section of the base of the heart with both atria and both aorta and pulmonic artery transected at their bases. FIG. 146 shows collar 1440 connected to tether 1442 which is in turn connected to main segment 10. FIG. 146 also provides a view of right ventricle 1460, mitral valve area 1461, tricuspid valve area 1462, aortic root 1463, and pulmonic root 1464.

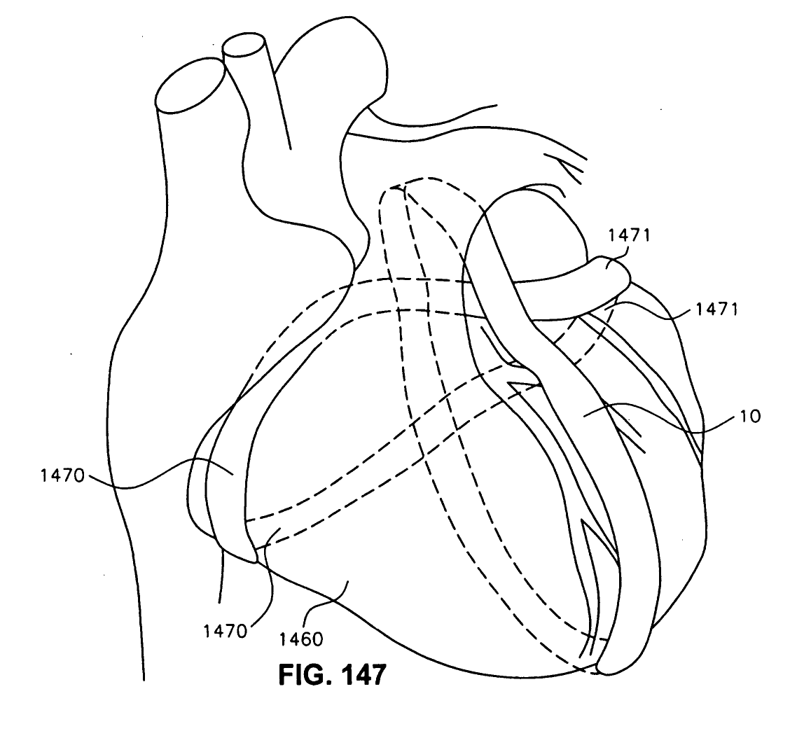

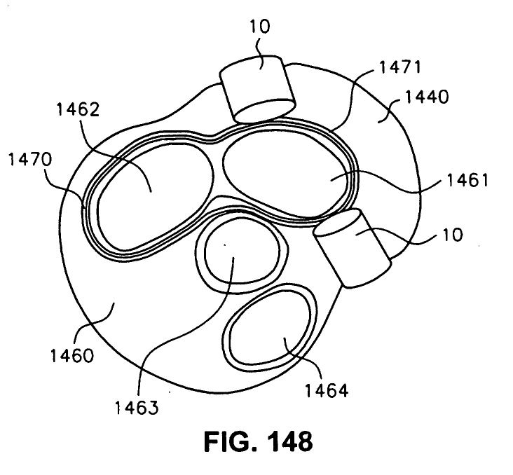

FIG. 147 shows a heart with a first band 1470 passing around the right atrioventricular junction, and second band 1471 passing about the left atrioventricular junction, where first and second bands may be stabilizer/reconfiguration segment 12 as described for example in FIGS. 5-8, or 68. FIG. 148 shows a top partial reduced cross-sectional view of the base of the view showing FIG. 147.

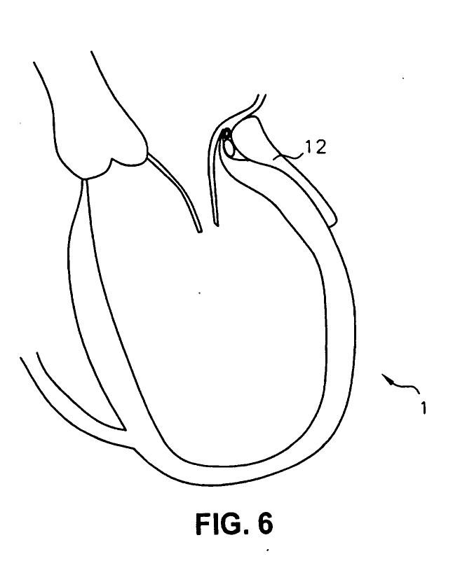

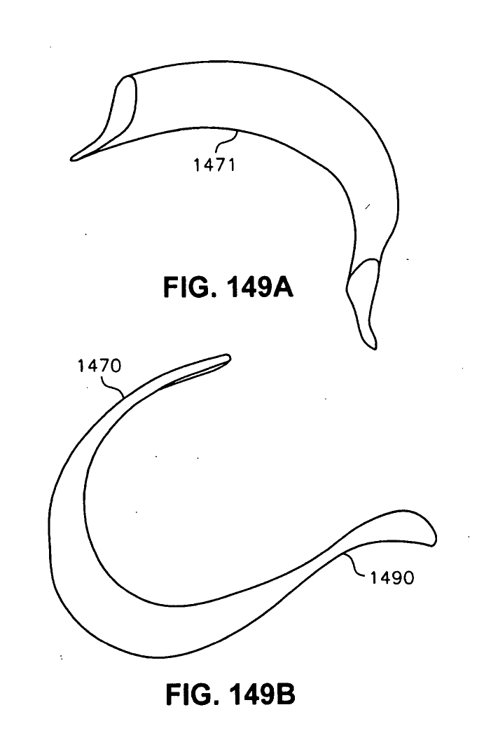

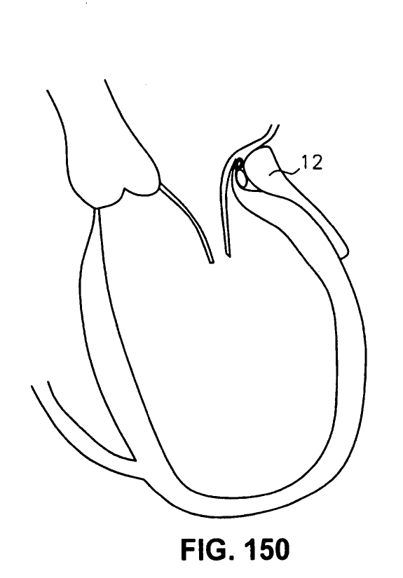

FIGS. 149A and 149B show bands 1470 and 1471, respectively, off the heart. In one embodiment, section 1490 of band 1470 is the narrow region intended to pass through the transverse sinus behind the aorta. In one embodiment, bands 1470 and 1471 are made generally of a low-durometer medical polymer, with a cross-sectional contour molded to the general shape evident from the cut ends in FIG. 149[b]a, as well as the cross section of stabilizer/reconfiguration segment 12 shown in FIGS. 6 and 150. The material used to form the device according to the present invention, particularly the major components thereof, is similar to a closed-cell foam such as neoprene, in terms of transverse stiffness and longitudinal flexibility. A fabric reinforcement may also be used or included in this element of the device. Also, bands 1470 and 1471 may include transverse stays and/or drawstrings for shortening adjustment, such as that which is shown in FIGS. 8, 10, 11A 11B.

Section 1490, which is intended to pass through the transverse pericardial sinus, is more nearly circular in cross section to match the anatomy in that location and to present a soft, blunt surface to underlie the right coronary artery. The overall width of band 1470 at their mid portion is generally about 10-30 mm, with a thickness of about 3 to 4 mm. Section 1490, in the area it passes through the coronary sinus is generally oval in cross section with a major axis of generally 8 to 10 mm and a minor axis of about 5 to 6 mm.

Band 1471 is shown in more detail in FIG. 149a. This band is generally similar the band 1470 described above, except this band has a relatively consistent cross-section rather than a variable cross-sectional section 1490 present on band 1470.

Aortic collar 1440 is a cylindrical cuff collar of, for example either fabric, low-durometer polymer, or both (that is, fabric-reinforced polymer). The length or height (dimension parallel to the long axis of the aorta) is generally about 10 to 12 mm, and the thickness is generally in the range of 1 to 3 mm. Edges of collar 1440 are softly radiused (as discussed above with respect to main segment [19]10) to minimize tissue trauma. It either has a tether 1442 as described above as an integral part, or [it] as some other connection (suture tab, snap eyelet, etc.) point for such a part.

One example for placement of aortic collar 1440 would be to insert a band of polytetrafluoroethylene (PTFE) felt around the aorta, with ends sutured together. Movement of collar 1440 would include following dissection of the pericardial reflections and connective tissue between the aorta and pulmonary aorta, using a procedure commonly used by those familiar with the art of cardiac surgery in the process of achieving hemostasis after aortotomy closure. In this embodiment, fixation of a band onto collar 1440 would be achieved by sutures or staples, done by methods known to those skilled in the art. In one embodiment, if tether 1442 were to be an integral part with collar 1440, a single band of felt or other fabric longer than the aortic circumference would be passed around the aorta, and one end connected (e.g., sewn or stapled) end-to-side to the remaining part, with residual length forming tether 1442.

In another embodiment, a premolded cylindrical collar made of fabric-reinforced low durometer biomedical polymer such as silicone rubber or polyurethane is divided at one point in the circumference and fitted with hooks or snaps for reconnection after passage around the aorta.

In another embodiment, a hinged rigid or semi-rigid polymer or metal collar that has a snap-connect or other fastening mechanism familiar to those know to those skilled in the art of restoring circular configuration after circum-aortic placement.

Tether 1442 is a flexible band or cord, for example made of braided polyester, joining collar 1440 to a main segment 10. The connection mechanism to main segment 10 can be any of those familiar to those skilled in the art, including sutures, screws, rivets, hooks, and snaps.

Another aspect of the present invention relates to that discussed above with respect to FIG. 69A. As noted above, FIG. 69A shows a cross-section in which main segment 10 is encased in a suturable material encasement 690 such as a polyester mesh, woven polyester, silicone rubber, polyester fabric or reinforced silicone. Encasement 690 about main segment 10 provides a means for attaching the straps 680 to main segment 10, which itself may be formed of material that would not accept a suture.

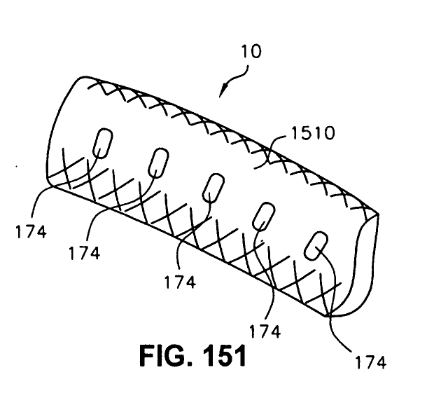

The present embodiment shown in FIGS. 151-158 uses an sheath or jacket (e.g., an elastic sheath or jacket) surrounding at least part of the device to be fixed adjacent to the heart wall. This aspect includes a method of locally fixing portions of a sheath or jacket to the epicardium, including fine sutures, adhesives, and mechanical fixation devices such as staples and clips, or combinations thereof.

FIG. 151 is a perspective view of a portion of a main segment 10 which is clad with an fabric sheath 1510 in accordance with the present embodiment. For this embodiment, stabilization protrusions 174 (such as shown in FIGS. 77a, 77b, and 77c) extend through openings in the sheath 1510.

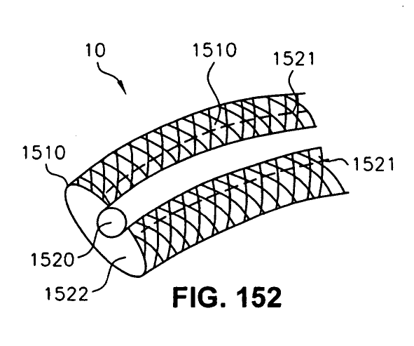

FIG. 152 is a perspective view of the device shown in FIG. 151, but from the outer (away from the heart) surface. Sheath 1510 is locally adhered (via form fitting or an adhesive or mechanical attachment) to the main segment 10 at discrete locations such as along parallel lines of attachment 1521. Segment 1520 is a backbone (e.g., a rigid rod) of main segment 10 that is to be attached to the heart in accordance with this embodiment, and pad 1522 is shown as covering segment 1520 to prevent segment 1520 from directly contacting the heart surface.

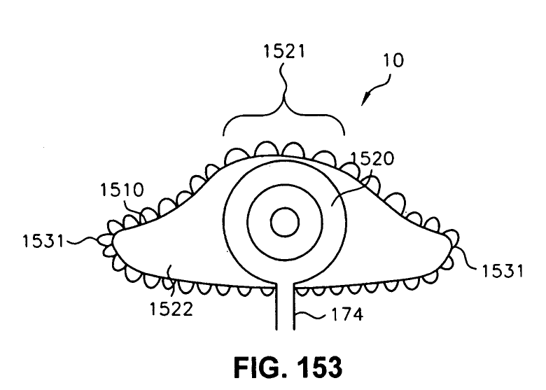

FIG. 153 is a cross section of the segment and sheath shown in FIG. 152. Stabilization protrusion 174 is shown in this view and is consistent with the disclosure above regarding delayed surface penetrating pegs shown in FIGS. 76A-82B. Outer edges 1531 of sheath 1510 (at the pad margin) are fixable (e.g., by adhesive, sutures, staples, clips, rivets, etc.) to the epicardium. Pad 1522 can be attached at the region of sheath 1510 that crosses the outer part of pad 1522, or, preferably, include a seam or fold to present a more convenient region for suturing, adhering, or stapling of pad 1522 to the epicardium.

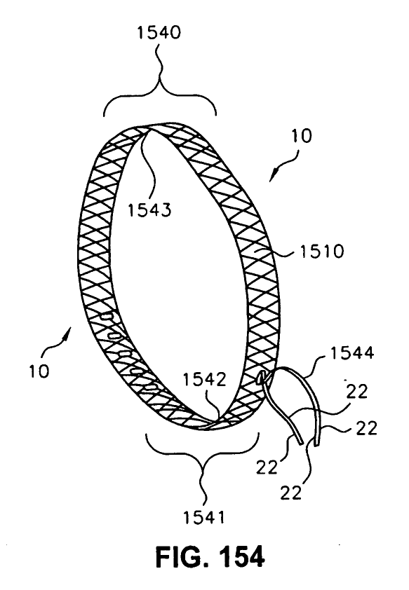

FIG. 154 shows a perspective view of the entire clasp according to one embodiment of the present application, including basal bridging section 1540 and apical bridging section 1541, both clad in a sheath 1510 consistent with the above disclosure, and two main segments 10. Sheath 1510 in this embodiment covers posterior main segment 10 and die bridging sections, basal section 1540 and apical section 1541. Sheath 1510 also covers anteroapical and anterobasal junctions 1542 and 1543, respectively, which are junctions between the basal section 1540 and apical section 1541 and main segments 10. Sheath 1510 can be used to cover one or more desired portions of main segment 10 and/or basal bridging section 1540 or apical bridging section 1541. Also shown are adjustment strings or cables 22 (as discussed for example with respect to FIG. 7) exiting from the anterior main segment 10 within sheaths 1544.

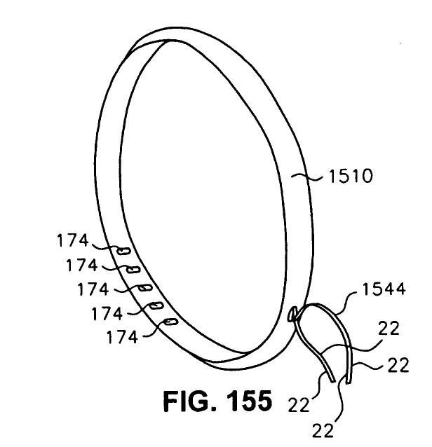

FIG. 155 shows the embodiment of FIG. 154 except that a dense sheath 1510, such as one made from polyester mesh of expandable PTFE (e.g., porous or non-porous), is shown. The density of the fabric can be changed by varying the degree of openness of the weave or net or porosity of the material. Sheath 1510 can be a porous, non-porous, woven or non-woven material.

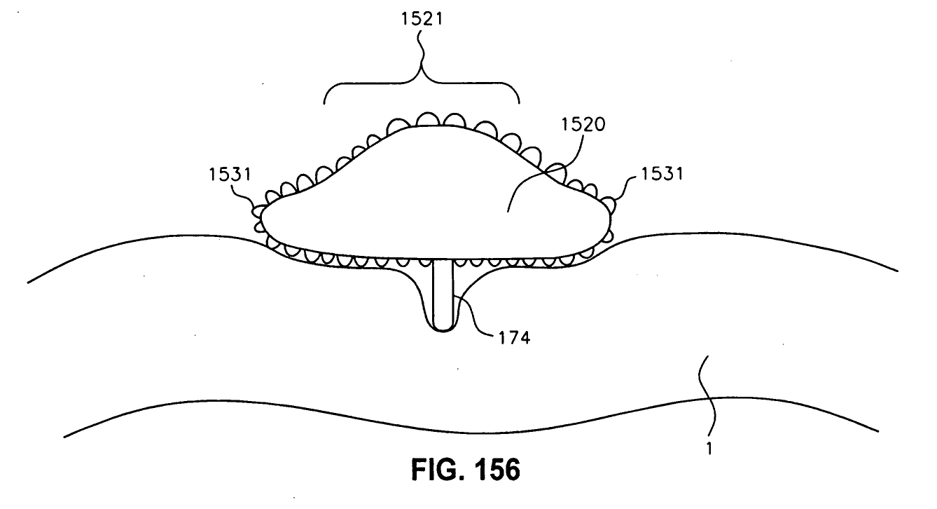

FIG. 156 is a cross section of main segment 10 according to one embodiment of the present invention, disposed on a heart surface 1 having sheath 1510 secured for example by suturing, adhesive, staples, clips, rivets, etc. at outer edges 1531, stabilization protrusion 174, and adhered to the main segment 10 at discrete locations such as along parallel lines of attachment 1521.

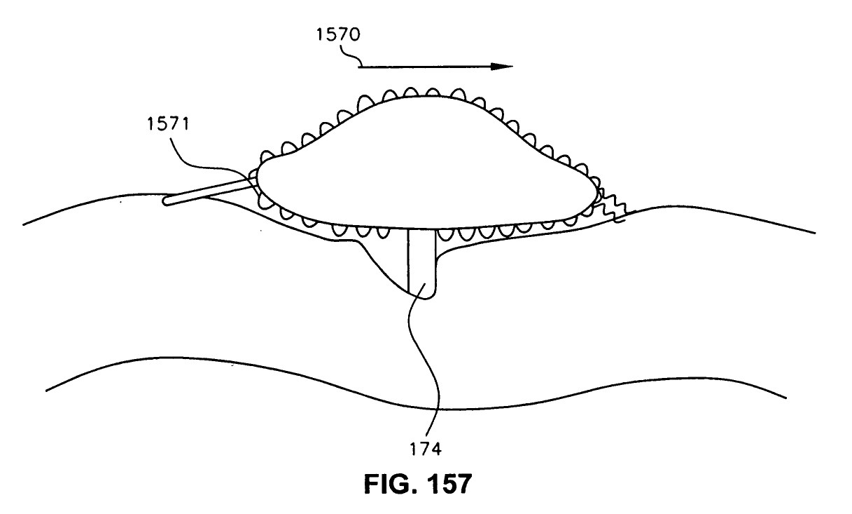

FIG. 157 is the same cross section as that shown in FIG. 156 and is offered to show the effect of a potentially displacing force from the left side (arrow 1570) of the device. Stabilization protrusion 174 is slightly displaced to one side of the epicardial indentation, and the point of fixation 1571 on the left is under tension.

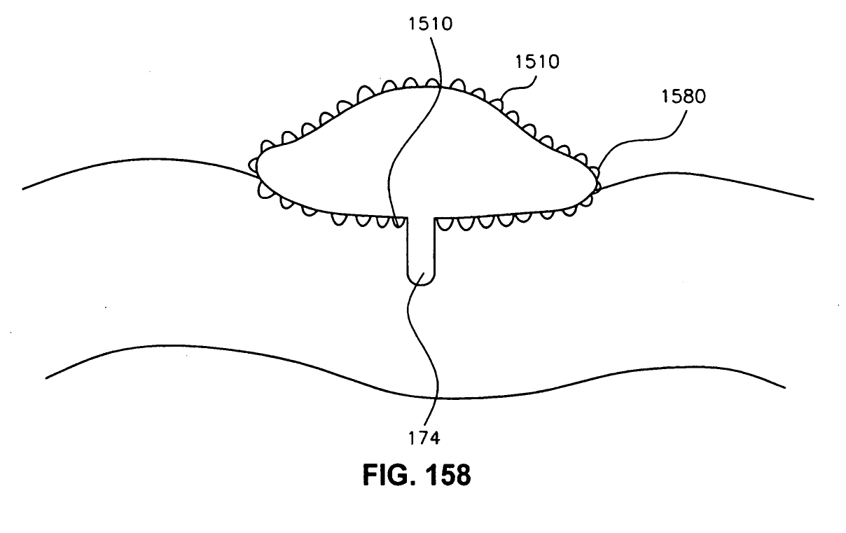

FIG. 158 shows the same cross section as that shown in FIG. 156, after penetration of stabilization protrusion 174 into the myocardium and tissue ingrowth has occurred into the sheath 1510 (for example a porous or mesh sheath), both at the points of fixation 1580 (e.g., with sutures, staples, clips, etc.) and elsewhere in the region of the epicardial contact.

Another aspect of the-present invention includes placement system for placing a heart clasp (including one or more main segments 10) such as that shown in FIGS. 2A-4, including three components which collectively join to dilate a delivery passageway and allow the introduction of a treatment device system. The dilator itself is removed at the end of the insertion.

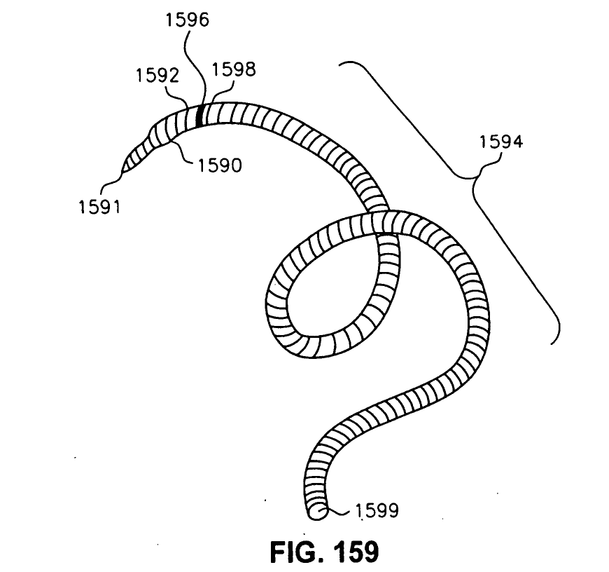

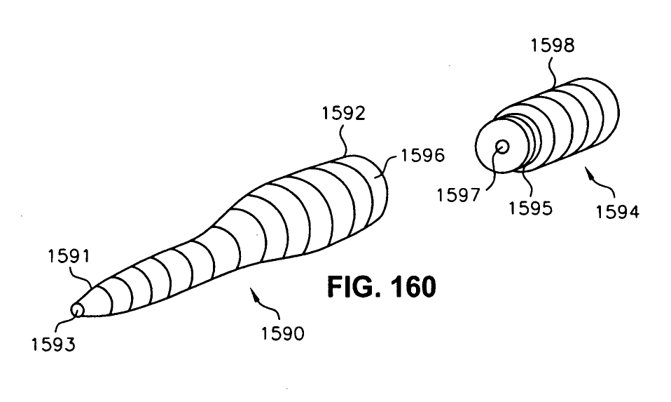

The first component is a dilator nose 1590 and is shown in FIGS. 159 and 160. Dilator nose 1590 has two ends, a tip end 1591 and a connector end 1592 opposite tip end 1591. Dilator nose 1590 is circular in cross-section, has a center channel or opening 1593 approximately 1 to 2 mm in diameter, is made of a soft elastomer such as polyurethane, and has a spiral wire reinforcement to discourage kinking and maintain flexibility. Dilator nose 1590 is tapered from a tip-end diameter only slightly larger than the center channel, to a diameter of approximately 15 mm at its connector end 1592. In FIG. 159, dilator nose 1590 is connected to a second component, the dilator body 1594.

FIG. 160 shows dilator nose 1590 separated from dilator body 1594. Dilator body 1594 has a threaded connector end 1595, which can be seen in FIG. 160. Dilator nose 1590 has an approximately 6 mm-long inside-threaded connector 1596 at its connector end 1592. Construction of dilator body 1594 is the same as that described above for dilator nose 1590, namely dilator body 1594 is formed from a soft elastomer reinforced with spiral wire and having a center channel 1597. Dilator body is approximately 30 to 40 cm in length, and has two ends, a nose[a body] connector end 1598 and free end 1599 (seen in FIG. 159). Outside threaded connector 1595 has the same length as the inside threaded connector 1596 described above in regard to dilator nose 1590.

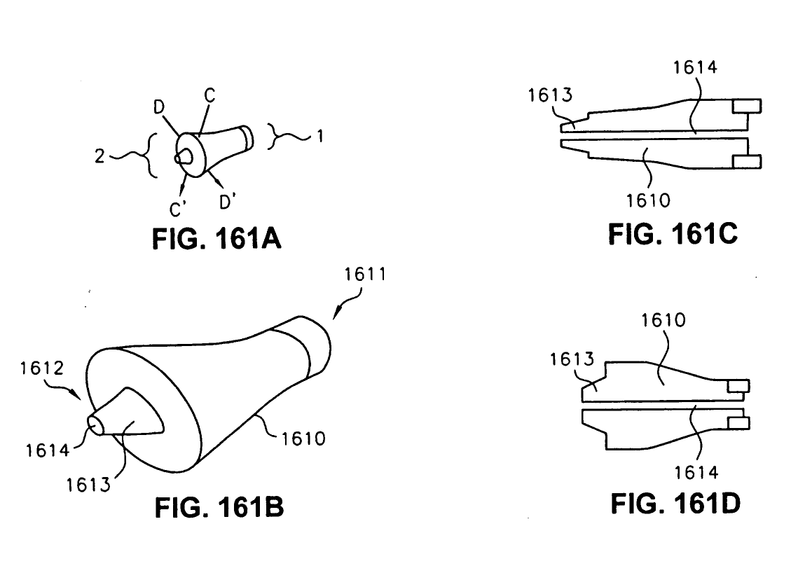

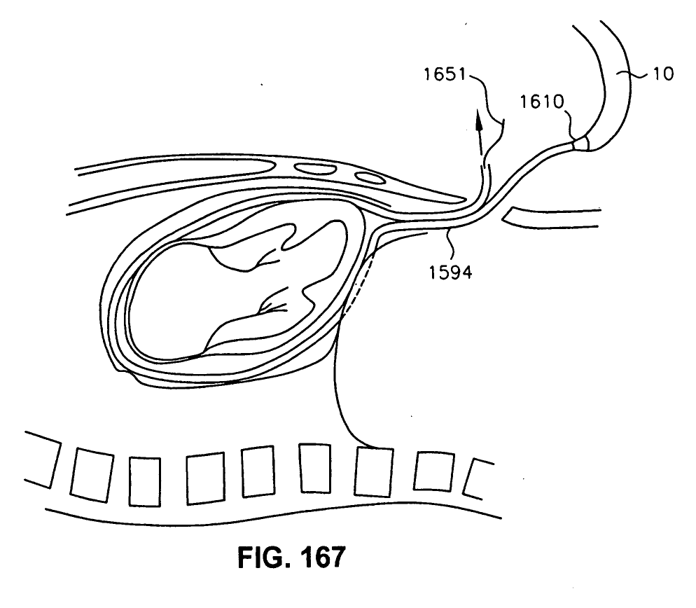

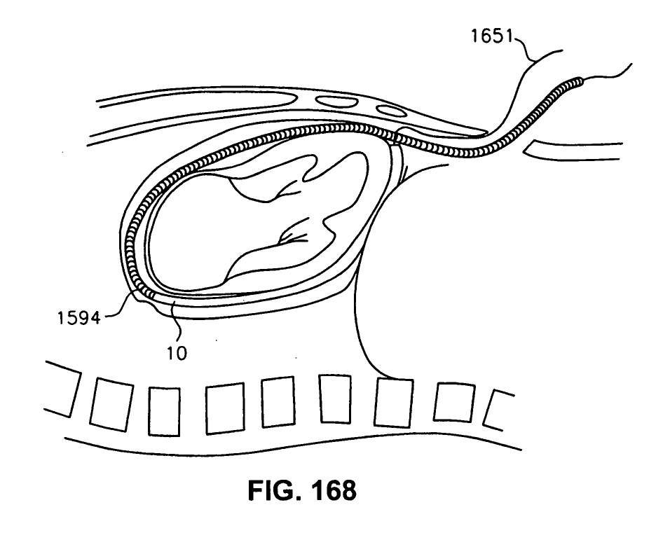

The third component is a dilator clasp adapter 1610 and is shown in FIGS. 161A-161D. Dilator clasp adapter 1610 has two ends, a dilator body connecting end 1611 and a clasp connecting end 1612 (such as for connecting to one end of main segment 10). Dilator body connecting end 1611 is circular in cross-section with a diameter the same as that of the body, and it is equipped with a threaded connector identical to that of dilator nose 1590. Clasp connecting end 1612 has a cross-section and dimensions similar to the clasp segment to which it is to be attached (shown in FIG. 167). In one embodiment, clasp connecting end 1612 is generally flattened, and wider in the direction tangential to the heart than in the direction normal to the heart surface. Clasp connecting end 1612 has a projection [1612]1613 that is elliptical in cross-section and tapered over its length. Projection [1612 ]1613 is intended to fit into a corresponding mating socket in the clasp segment to which it is to attach, so that the clasp segment will not rotate on its long axis after attachment. As shown in FIGS. 161C and 161D which are taken along lines C-C′ and D-D′, respectively, in FIG. 161A, dilator clasp adaptor 1610 includes a channel 1614 for accommodating a guidewire (not shown).

A method of using several devices according to the present invention is shown in FIGS. 162-170. FIG. 162 shows a schematic representation of a heart located in a chest cavity. FIG. 162 shows that a small incision has been made into the subcutaneous tissue of the upper abdomen wall at point 1624, just below the lower rib margin, near the xiphoid process (that is, the or xiphisternum or the lowest part of the sternum or ‘breast bone’). Then, using blunt and sharp dissection the junction of the abdominal wall muscles and diaphragm is exposed and opened. Next, the pericardial sac is opened. The tip of a sterile flexible fiberoptic endoscope 1620, such as a bronchoscope, is introduced into the pericardial cavity, and, with visualization through the scope [1625]1620, advanced behind the left ventricle 1621 and then behind the posterior wall of the left atrium 1622. Note that although FIG. 162 shows an eyepiece [1625]1626 for illustration, the endoscope will typically be equipped instead with a video camera and image shown on a monitor as the surgeon advances the endoscope, allowing sterility to be maintained. Other structure shown is sternum 1623.

FIG. 163 shows a view as endoscope 1620 reaches the superior limits of the pericardial pouch called the ‘oblique sinus’. The four pulmonary veins (1630, left inferior; 1631, left superior; 1632, right inferior; and 1633, right superior) flow into the posterior wall of the left atrium 1634). The inner surface posterior wall 1635 of the pericardial sac is also shown.

FIG. 164 shows a biting forceps 1640 of the type used for bronchial biopsies, advanced through the channel of endoscope 1641. The jaws of forceps 1640 are shown grasping pericardium 1635, cutting a hole 1642 in it. In this procedure, it is preferred to stay well away from the posterior wall of the left atrium 1634.

In FIG. 165, endoscope 1620 has been advanced through this hole, around the front of the left atrium and ventricle, and back out the entry site into the subcutaneous incision, all under direct vision through scope 1620. This guidance may or may not be aided with additional visualization, such as that provided by a thoracoscope via another port in the side of die chest, or x-ray fluoroscopy, both using methods familiar to those skilled in cardiac surgery. A forceps is then used to grasp an approximately 1-mm diameter tether or guide wire 1651 (which may be polymer cord, metallic cable, or similar flexible material as disclosed above) to pull this tether back around the path that had been negotiated by endoscope 1620.

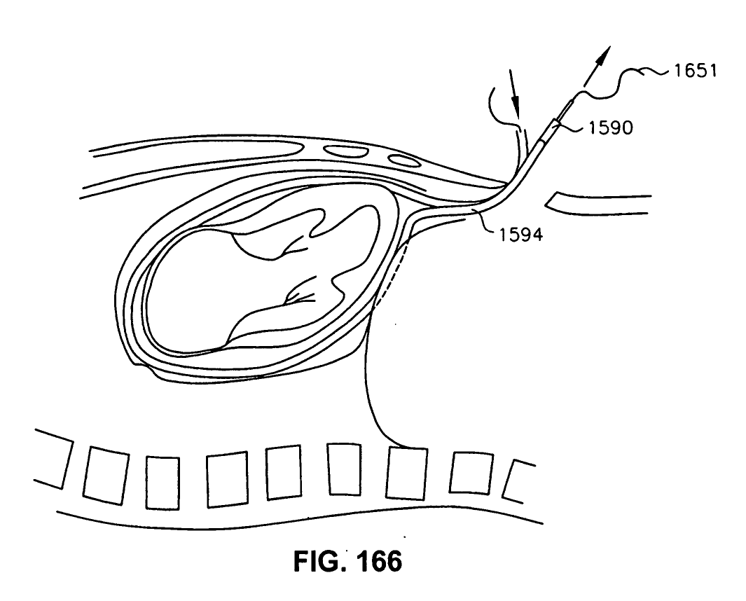

FIG. 166 shows the dilator 1594 and 1590 (body and nose components) advanced over tether 1651.

In FIG. 167, dilator nose 1590 has been detached (unscrewed) from dilator body 1594, and dilator-clasp adaptor 1610 (as shown in FIG. 161) has been attached to the dilator body 1594. Tether 1651 end that passes through the connector is advanced through a tether-channel in the apical-posterior-basal portion of the main segment 10 and temporarily fixed at the opposite end of this portion. Traction on the dilator and the opposite end of the tether 1651 then pull main segment 10 between the posterior wall of the heart and the posterior wall of the pericardium.

FIG. 168 shows the apical-posterior-basal portion including a main segment 10 of the clasp in its intended position in back of the heart.

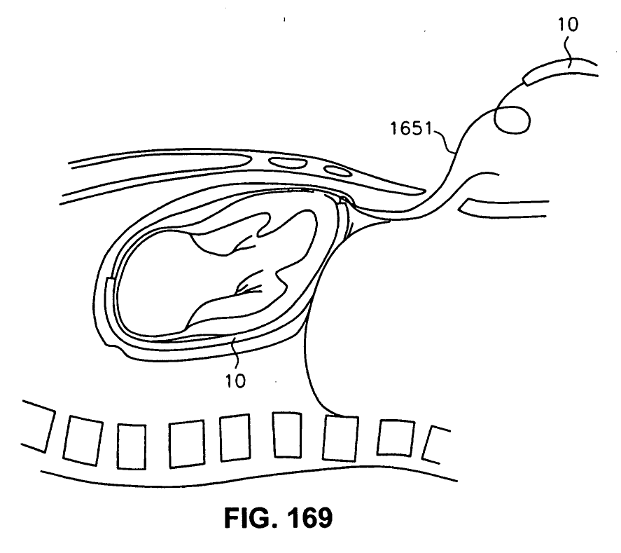

FIG. 169 shows tether 1651 being threaded into the superior end of the anterior portion of a second main segment 10 of the clasp, after dilator 1594 and dilator-clasp adaptor 1610 having been withdrawn from over tether 1651.

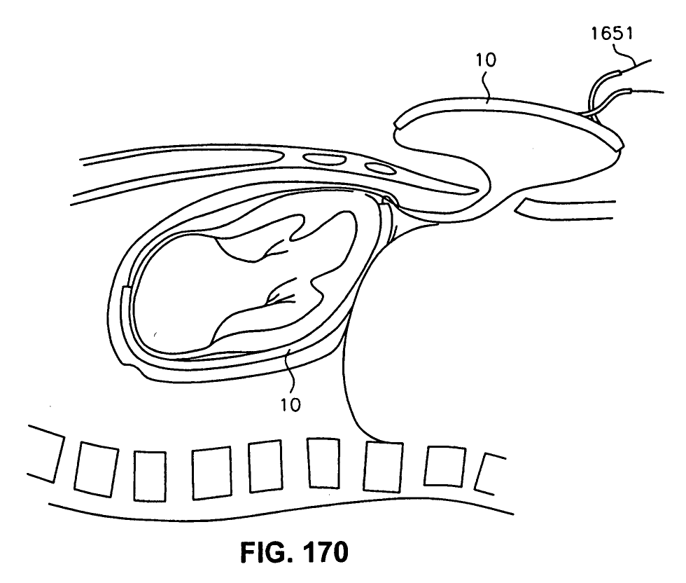

FIG. 170 shows the anterior portion including main segment 10 of the clasp with both ends of the tether 1651 threaded through its channels of main segment 10.

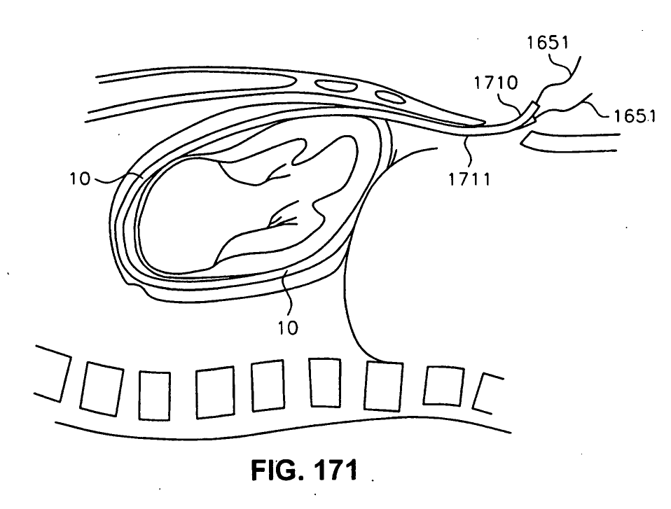

FIG. 171 shows the clasp including two main segments 10 in place, portions labeled as in FIGS. 169 and 170, with die tether 1651 (optionally in outer sheaths 1710 and 1711) in tether channels (no shown) on or in the clasp and extending into the subcutaneous incision. At this point, tether channels and tether 1651 ends may be connected to any adjusting and locking mechanisms discussed above, that are designed for use with the clasp in accordance with the present invention.

Another aspect of the present invention relates to that which is disclosed above with regard to clasp placement or fixation. In this embodiment, areas of hook and pile type Velcro® fasteners or similar reusable and removable fasteners, in a biocompatible material, are fixed, directly or indirectly as parts of a patch that is to be attached to the epicardium. Mating areas of hook and pile type Velcro® fasteners are part of a composite sheath within which the to-be-mounted structure is clad.

The type of Velcro® fastener selected (in terms of distribution) is such that die desired degree for freedom of placement and readjustment is obtained. Corresponding Velcro® fastener strips placed on the heart and the device may be parallel or perpendicular to one another.

Regions of Velcro® fasteners can include more elastic, fabrics of near equal thickness and thickness-compliance are combined so that lateral elasticity of these flexible composite structures is maintained. This is employed in construction of both the epicardial layer (containing hook and pile type Velcro® and more elastic fabric) and the sheath that is place about the to-be-mounted structure or structures.

More specifically, securing one side of the Velcro® fastener to the epicardium is generally done by multiple discrete fixation points, whether superficial (epicardium) sutures, rivets, cements, or very superficial staples, so as not to preclude segmental shortening or relaxation of the subepicardial myocardial layers. Securing other side of the Velcro® fastener to the to-be-mounted clasp segments (e.g., main segments 10) is similarly kept localized, generally on a surface not in contact with the heart (outer surface), along a single line perpendicular to the direction of maximal wall contraction (circumferential)—i.e., the center line of a vertical structure—or both.

A pattern of patch construction using 4-5 mm wide vertical (relative to the heart) strips of hook and pile type Velcro® fastener alternating with 5-7 mm wide strips of far more elastic polymer knit or weave, joined by flat stitching, and a similar sheath material, including alternating 34 mm wide Velcro® fastener and 4-5 mm wide elastic polymer in the structure sheaths, are non-limiting examples of such a system.

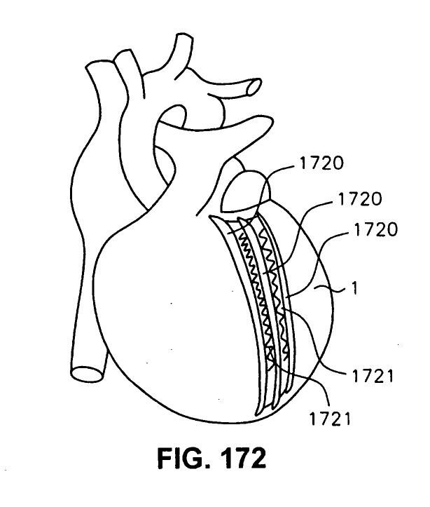

As an example, FIG. 172 shows a heart 1 with a composite patch including one side of Velcro® fastener and elastic polymer knit or weave is sewn to the surface of the heart. Strips of one side of a hook and pile type Velcro® fastener 1720 are adjacent but separated by interposed strips of elastic fabric 1721 having a thickness approximately equal to the strips of Velcro® fastener 1720.

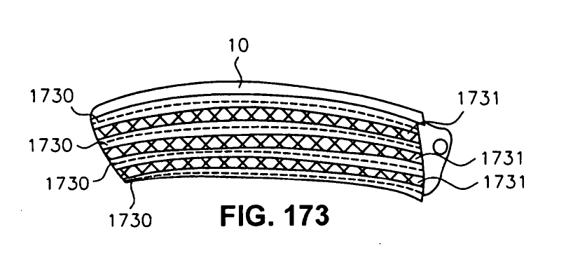

FIG. 173 shows an enlarged view of a second side of a hook and pile type Velcro® strip which can be adhered to a heart contacting surface of a clasp bar (such as main segment 10) or other member to be attached to the heart. In one embodiment, this patch is comprised of interposed rows of strips of hook and pile type Velcro® fastener 1730 and strips of elastic fabric 1731 of similar thickness.

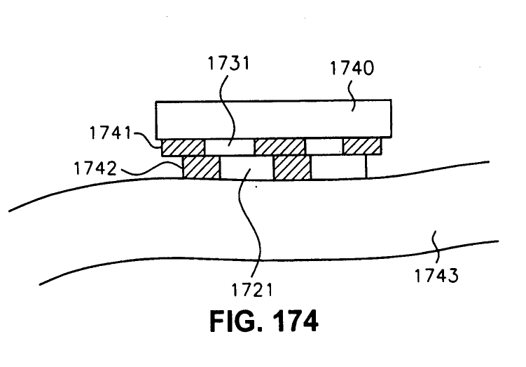

FIG. 174 illustrates a section of a heart wall and its attached structure interface where 1740 is the attached structure (such as main segment 10), 1741 is one layer of hook [and]or pile type Velcro® fastener with interposed row of elastic fabric, and [1741]1742 is [the other]a layer of [a hook and pile]the opposite type (pile or hook, respectively) type Velcro® fastener with interposed rows of strips of elastic, and 1743 is the heart itself. Elastic strips allow some movement of Velcro® fastener longitudinally and laterally

The embodiment described above allows securing of prosthetic-tissue fixation without a precise determination of the final location of the prosthetic structure because a subsequent special determination can be decided after fixation of the Velcro® fastener containing epicardial strip. After that special determination is made, the structure can be removed, have its position altered, and replaced later in the operative procedure. In addition, this embodiment adds the benefits of (a) safe readjustment of position, and (b) more unobstructed, and thus likely safer, access to epicardial fixation points than that of either direct rigid-structure placement or attachment via a pre-mounted elastic sheath.

While the invention may be embodied in many different forms, there are shown in the drawings and described in detail herein specific preferred embodiments of the invention. The present disclosure is an exemplification of the principles of the invention and is not intended to limit the invention to the particular embodiments illustrated.

What is claimed is:

1. A device for treating a diseased heart, said device comprising:
one or more members configured to surround a selected portion of the heart, including a first member configured to be positioned adjacent an exterior surface of one chamber of the heart and to selectively deform the chamber by pressing inwardly thereon, and
a second member coupled to said first member, and configured (a) to lie adjacent an external surface of the heart in a path forming an angle with said first member and (b) to stabilize said first member on the heart.

2. A device according to claim 1, wherein said second member is a segment configured to selectively deform a portion of the heart.

3. A device according to claim 1, wherein at least a portion of said second member is a segment configured to lie adjacent the valvular annulus of the heart.

4. A device according to claim 1, wherein at least a portion of said second member is configured to lie adjacent the papillary muscle of the heart.

5. A device according to claim 1, wherein at least a portion of said second member is configured to lie adjacent the left ventricle of the heart.

6. A device according to claim 1, wherein at least a portion of said second member is configured to lie adjacent the right ventricle of the heart.

7. A device according to claim 1, wherein said second member includes a porous segment.

8. A device according to claim 1, wherein said second member includes a lattice structure.

9. A device according to claim 1, wherein said second member includes a segment configured to have an adjustable length.

10. A device according to claim 1, wherein said second member is rigid.

11. A device according to claim 1, wherein said second member is semi-rigid.

12. A device according to claim 1, wherein said second member is flexible.

13. A device according to claim 1, wherein said second member includes a segment configured to be secured to a lumen of the heart.

14. A device according to claim 1, wherein said first and second members are integral with one another.

15. A device according to claim 1, wherein said second member is a protrusion.

16. A device according to claim 15, wherein said protrusion is a peg.

17. A device according to claim 15, wherein said protrusion is blunt.

18. A device according to claim 15, wherein said protrusion is resorbable.

19. A device according to claim 15, wherein said protrusion is partially resorbable.

20. A device according to claim 15, wherein said protrusion is non-resorbable.

21. A device according to claim 15, wherein said protrusion includes a non-resorbable porous element.

22. A device according to claim 1, wherein said second member is a protrusion configured to penetrate a surface of the heart over a predetermined period of time.

23. A device according to claim 1, wherein said second member is a protrusion configured to move relative to said first member and to a surface of the heart.

24. A device for treating a diseased heart, said device comprising:
one or more members configured to surround the heart, including
a first member configured to be positioned adjacent an exterior surface of one chamber of the heart and configured to selectively deform the chamber by pressing inwardly thereon, and
a second member configured to stabilize the first member of the device in a preselected position on the heart, said second member comprising a facing material on at least part of one side of at least one of said first and second members, and facing the exterior surface of the heart,
said facing material being configured to facilitate epithelial growth into said facing material.

25. A device according to claim 24, wherein said facing material is porous.

26. A device according to claim 24, wherein said facing material includes a protrusion.

27. A device according to claim 26, wherein said protrusion is a molded projection.

28. A device according to claim 24, wherein said facing material includes a sheath configured to surround a portion of said first member.

29. A device according to claim 28, wherein said sheath is porous.

30. A device according to claim 28, wherein said sheath is elastic.

31. A device according to claim 28, wherein said sheath is configured to be secured to an external surface of the heart.

32. A device for treating a diseased heart, said device comprising:
one or more members configured to surround a selected portion of the heart, including a first member configured to be positioned adjacent an exterior surface of one chamber of the heart and to selectively deform the chamber by pressing inwardly thereon, and
a second member coupled to said first member, and configured (a) to lie adjacent an external surface of the heart in a path with said first member and (b) to stabilize said first member on the heart.

33. A device according to claim 32, wherein said second member is configured to lie adjacent an apical portion of the heart and to accommodate a portion of said first member.

34. A device according to claim 33, wherein said second member is a conical.

35. A device according to claim 33, wherein said second member is configured to have an adjustable size.

36. A device according to claim 33, wherein said second member includes at least one protrusion configured to accommodate a portion of said first member.

37. A device according to claim 36, wherein said protrusion is a channel.

38. A device according to claim 33, wherein said second member is rigid.

39. A device according to claim 33, wherein said second member is semi-rigid.

40. A device according to claim 33, wherein said second member is flexible.

41. A device for treating a diseased heart, said device comprising:
one or more members configured to surround the heart, including a first member configured to be positioned adjacent an exterior surface of one chamber of the heart and to selectively deform the chamber by pressing inwardly thereon, and
a second member configured to stabilize said first member of said device in a preselected position on the heart,
said second member comprising a first adherent surface on at least part of an inner side of said first member, facing the exterior surface of the heart.

42. A device according to claim 41, wherein said second member further includes a second adherent surface secured to an exterior surface of the heart for releasably attaching said first adherent surface.

43. A device according to claim 42, wherein one of said first and second adherent surfaces includes at has at least one hook and said other adherent surface includes uncut pile for releasably receiving the hook.

44. A device according to claim 42, wherein at least one of said first and second adherent surfaces is at least partially elastic.

45. A device according to claim 42, wherein said first adherent surface includes an adhesive.

46. A device for treating a diseased heart, said device comprising:
one or more members configured to surround the heart, including a first member configured to be positioned adjacent an exterior surface of one chamber of the heart and to selectively deform the chamber by pressing inwardly thereon, and
a second member configured to stabilize the first member of said device in a preselected position on the heart,
said second member including one or more elements configured to penetrate an exterior surface of the heart.

47. A device according to claim 46, wherein said second member includes protrusions configured to penetrate only an outer part of the exterior surface of the heart wall.

48. A device according to claim 47, wherein said protrusions are configured to be retained within the heart wall.

49. A device according to claim 46, wherein said second member includes protrusions configured to penetrate through the exterior surface of the heart wall and to be retained on an inside surface of the heart wall.

50. A device for treating a diseased heart, said device comprising:
one or more members configured to surround the heart, including a first member configured to be positioned adjacent an exterior surface of one chamber of the heart and to selectively deform the chamber by pressing inwardly thereon, and
a second member configured to stabilize the first member of said device in a preselected position on the heart, said second member including one or more elements attached to said second member at spaced locations and configured to pass through the exterior surface of the heart.

51. A device according to claim 50, wherein said elements are sutures.

52. A device for treating a diseased heart, said device comprising:
a first member configured to contact a surface of a chamber of the heart and to continually bias a wall of the heart, and
a second member connected to said first member and configured to stabilize said first member in a preselected location in contact with the surface of the chamber.

53. A device according to claim 52,
further comprising a third member connected to said first member and configured to be positioned on an exterior surface of the chamber and to selectively deform the chamber.

54. A device according to claim 52, wherein said first member is a spring.

55. A device according to claim 54, wherein said spring is a helical spring.

56. A device according to claim 54, wherein said spring is a leaf spring.

57. A device according to claim 54, wherein said spring is a coil spring.

58. A device according to claim 54, wherein said spring is a flat spring.

59. A device according to claim 52, wherein said first member is configured to lie inside a chamber of the heart.

60. A device according to claim 52, wherein said first member is configured to lie outside a chamber of the heart.

61. A device according to claim 52, wherein said first member is configured to lie inside a wall of a chamber of the heart.

62. A device according to claim 52,
further comprising a biocompatible sheath covering a portion of said first member.

63. A device according to claim 52, wherein said second member is configured to lie adjacent an apical portion of the heart and to accommodate a portion of said first member.

64. A device according to claim 1,
further comprising a transceiver coupled to one of said first member and said second member for receiving and transmitting electronic signals to and from said device.

65. A device according to claim 1, wherein said first member includes a plurality of elements pivotally connected to said first member, wherein said elements are configured to maintain a tangent position on a surface of the heart.

66. A device according to claim 65, wherein said elements are rigid.

67. A device according to claim 65, wherein said elements are semi-rigid.

68. A device according to claim 65, wherein said elements are flexible.

69. A device according to claim 65, wherein said elements have an edge and said edge has a radius of curvature of between 0.2 mm and 10 mm.

70. A method for placing on a diseased heart a device including a tether having two ends, said method comprising the steps:
passing the tether along a predetermined line of approximate placement position on the heart of the device,
attaching a first portion of the device to one end of the tether,
pulling a first portion of the device into approximate placement position with the tether,
attaching a second portion of the device to the second end of the tether,
sliding the second portion along the tether and placing the second portion of the device into approximate placement position abutting said first portion, and
connecting the two portions to one another.

71. A method according to claim 70, further comprising the step of passing the tether and a portion of the device through an opening in a pericardial reflection of the heart.

72. A method for placing on a diseased heart a device including a tether having two ends, said method comprising the steps:
passing a tether having two ends along a predetermined line of approximate placement on the heart of the device,
sliding a sheath over the tether,
attaching one end of the sheath and one end of the tether to a first portion of the device,
pulling the first portion of the device into approximate placement position on the heart,
disconnecting the sheath and sliding the sheath off the tether,
attaching a second portion of the device to the tether,
sliding the second portion along the tether and placing the second portion of the device into approximate placement position, and
connecting the two portions to one another.

73. A method according to claim 72, further comprising the step of passing the tether, sheath and a portion of the device through an opening in a pericardial reflection of the heart.

74. A method for placing a device in a diseased heart, the device including a first automatically reversibly collapsible anchor and a first tether attached thereto, and a second automatically reversibly collapsible anchor and a second tether attached thereto, said method comprising the steps:
passing a sheath through a lumen into an interior portion of a chamber of the heart,
sliding the first collapsible portion in a collapsed position through the sheath and through a first predetermined portion of a wall of the chamber and causing the first collapsible anchor to expand,
sliding the second collapsible portion in a collapsed position through the sheath and through a second predetermined portion of a wall of the chamber and causing the second collapsible anchor to expand, and
connecting a free end of the first tether to a free end of the second tether.

75. A method for placing on a heart a device for encircling the heart and for pressing inwardly thereon, the device included a plurality of elongate elements adapted to be joined successively with one another and, when joined, to surround the heart, said method comprising the steps:
placing a guide member in a path around the heart, the path corresponding generally to a pre-selected location surrounding the heart in which the joined elongate elements are intended to be located,
guiding one or more of the elongate members along the guide member to the preselected locations of each of the elongate elements on the heart, and
after two of the elongate members are in their respective pre-selected positions, joining the two elongate members together.

76. A method according to claim 75, wherein the guide member is a tether configured to pull the elongate elements along the path.

77. A method according to claim 75, wherein the guide member is a tubular member configured to pull the elongate elements along the path.

78. A method for introducing a transventricular tension member between substantially opposing walls of a heart chamber and anchor members on each end thereof, the anchor members being expandable from a compressed configuration in which the anchor is confined to a relatively small diameter to an expanded configuration in which one end of the anchor is expanded to a relatively larger diameter including a relatively planar surface, and the anchor member is attachable to a tension member extending away therefrom, said method comprising the steps:
endoluminally introducing a first anchor into an interior of the chamber in the compressed configuration and causing the first anchor to pass through a wall of the chamber to the exterior thereof,
causing the first anchor to expand to its expanded configuration with the planar surface resting against an exterior surface of the chamber wall,
endoluminally introducing a second anchor into an interior of the chamber in a compressed configuration and causing the second anchor to pass through a wall of the chamber to the exterior thereof,
causing the second anchor to expand to its expanded configuration with the planar surface resting against an exterior surface of the chamber wall, and connecting the first and second anchors to a tension member.
